SEQUENCE LISTING (321 sequences)

SEQ ID NO: 1 - DNA, 61000 bp, Homo sapiens
  source 1..61000: mol_type = genomic DNA
actggattcatccattttgaatcattttcaacactgtattatttattatttcacaaattgtttttaactgtctaccacgtgaatcatgaaaccaaccactaggaagggtataaagatatacactattcattctctacctttcaaaggtttaaatgtattggaaacactaaatatagtcatagtacttaaaattactatatatccatgatggcttagacagttcatacatgataacccttcagtctacccaaatccccacacgacagatactggttgtccttagtttatactgtgcaaatgtagtacctattctttgagtgtccacccattcccatcaaaaccatttccttcaggaaaagctgatctcacctccagacctctagggccaactctatctatatggttagacgaaacagcaaatgcacttttattattagtgccagtttgggttattttttcaaatttttgcaacaaagtgtgatccacaatacacaagatcatattcactcccccaaattaatattttcttaaagagatgggtgttatattttccttcactttctatttttcagacattaaggagaagcttggctggcatcacaaaagcctgaaacatcatcaaataaatatttgttgaataaattagctgaacaaatgaataagtgaataacatattgaatatgaaataatttttctgttcatatttcacccttactttcccatgtgaatatgcatgataccatattgtaatgataaaagaaaacaaaaaacaatttgataatggcacagccctttgaatttatttgtgttttcttagctgattgacaattttgcctactatctttttattctatggtaaatcatattgacctactctcttccctgtttgacattatttaaaaatcattcataactgaagttaggtggctaacacctagaaaatcggaaatagaaaagagtcacatatgtgacccttagggcagtagaatgagtgttttgttttcctcatagtcatcaaaagtgcaatcgatttcattcagcagccaactgcagttgttaaagaaactaatgccatgacttagggcccttgttctccaaggaagctgaagaatgtgtagagatgacacacagcctgaggggaaagggtgtggtggggagaagagaaatactttgaaaatattagtcacacagacatgctgaagatccttcacctcatgagtgataactactccagtgagaggtgattaactgttacatttaaagtgggaaatgcctatatatagagagaaaatggttatttatttccagaccctgccaacttccatattgctctcttgctgtgtcatagtcacagacctgttcaaagataggaaatataatttctaatcttcctgttcaagaaataattttattgatctccacttcatcttttattttccccatgattcagcaggtcaaaactgttccagttccagtagcatcctatcagtcagcaaaatgtccatgaggttaaaatgtcttacaagttaataggaggtgaagaaattcatttcagcaccaaggagagtgccctgctattttcaattggttttatgtatttgaaactgtgtattgttcctgaggatagaaaaaaaagttgaaatgtgattggacagaaagctgctaaaaacctattttaataagaagaaaaaaatacattcatttttctgaaattaaaaacttgccagggtttcatacctgttcatgattgcaacaatgtgctcttcttttggctttcaaacgtcggaattagcaattctttgcctatttttctaatattaaaaccagtttctgcatggaattagactggctcaataaagttacagaagagttaaattagcagtaattaaaaacttcatgcagcaactcctaaactttttctcaccattttcagagctgtgcacatggtaaatgctcaatacatatttaagtcaagtgaaagagaacaaatgaatgactggcatccattaagtgagaaggaggaactaataaatgccttgtttgtaatgaaagtcattttttgcaactagtctcaaagatcctcacatacgacattgctcaatccgaaacttgaacacagccttcctgggcctacataaacgtagtctctctggatctcagaaaaataatgacccagaccacacctttcatctcttctaagttttccatgtaaagcattctccttagagccctggaaaggttactaaaattttgacccacttaatcctacaccttcagggcaacatggcattcacctgtggtttccttaatgcccaaaccagccagccaacctgttttctgccgtgttaacaaatggacaggacacaggcaccagaggttaaactttcttcctcttcccccatgtccccaggtggaccattattaccatccctattcccaagcaaccaggtagccccttcctctgtgccaagggaagaaaacaccatgcttccctctcctgagagaccttcaagagtttagagacgcctaagtccagctgtgctaaaagataaaggacaataatgcaagtctgctctgagctgccaccatagccagacatagggtagccctgaaagactagaaccaaggacagagccaaaggtgaaagaaaatatgaaaaagtgaaaacacagtatttaaacagaattttccaacagcctgcatatagtgaggacttctcaagcttctgaatccttttccattgaattgtgcaatggcacatgtatgagcaaagtcaagcctcctggctcccaggtaggcacagcccagttcttagctcctaggaagcttcagggcttaaagctccactctacttggactgtactatcaggcccccaaaatggggggagccgacagggaaggactgatttccatttcaaactgcattctggtactttgtactccagcaccattggccgatcaatatttaatgcttggagattctgactctgcgggagtcatgtcaggggaccttgggagccaatctgcttgagcttctgagtgataattattcatgggctcctgcctcttgctctttctctagcacggtcccactctgcagactcagtgccttattcagtcttctctctcgctctctccgctgctgtagccggaccctttgccttcgccactgctcagcgtctgcacatccctacaatggctaaaacagcaatgggtaaggcactgcgcctcgttctccgtcggctctacctggagcccacctctcacctcctctcttgagctctagaagcattcagagatattttataaagaaaaagatgttaatggtaacacaggaccaggaaggacagggcagttctgggggaggtgggagggcagagaagaggtctatggaaatctaaagcgaagaatttcttttaaaaggtagaagcgggtaagttgccctcctatgggtagagaatttattctgtttccatatttaaaattaggactcaatcgtgaggggaggaagctaccttaactgtttgccttaaatgggcttaagggacattttggaaagtgctttataacgaccttttttttttttatttcttctctagtttaagaagaaaataggaaaggggtaaagggaaggtgggagaaaggaaaaagaaaattgcaaagtcaaagcggtcccatcccgctgtttgaaagatgggtggagacggggggaggggatggagagaactgggcacattttacggtattgtctcgtcgaagaaaccgctagtcctggggtgcggtgcagggaggtaagacggcgggggacagggtgggggtaggacctccgctcctttgttttagggcaagggaggggaaggagagaggaagtcgcggagggcgtggagggcgcgggtgggcagctgcaggggcggggaagcgcgcggcagggaggggtggagggacagcggcttcgaaggcgctggggtggggtttctttgtgtgcggaccagcggtcccggggggaggcacctgcagcgctgggcgcacaatgcggacagccccacccagtgcggaaccgcgcagccccgcccccccgcccggtgctgcatcttcattcgaaagggggtcgggtggggagcgcagcgtgacacccaggagcccaaccctgcggggacagcggcgccacgccccgcgctccccgctcccgactccccgccgcggcttccaagagagacctgaccactgaccccgccctccccacgctggcctcattgttctgcttttaagagagatgggaaaagtgggttaacatttttcttttcggaagcaaattacatagagtgtttagacatagacacagataaagggttctttgaagacctttgatcgtttgcgggaaaagcttctagaacctagacatgtgtatgtataataatagagatgacatgaaatcgtatataaagcaaaagaggtcaaagtcttaagttaagccacgcgaaatttccgttttgtgggtcagacagtgccaaatatcggcaatttcataagctcagagagacaagacagtggagacacaggatgaccggaaaagattctggattcagggccttcatccgcaattggtcttgtgccttgagtgcccacggttctggcgctcagtggccccggggtgaaaaggcagggtggggcctggggtcctgtggcagctggaagcacgtgtcccccgggacttggttgcaggatgcggagacagggaaagctgccgaaaggactccatctgcgcggctccgccctgccctaccctccccgcggagccggggagacctcaggctccgagactggcggggaagaggaatatgggaggggcagttgagctgtatgcagtcctggaacctcttttttcagccccgcagtccacaacggcccgagcaccccttgatgtgcgcagacccccggcgtggctctcagccccagcaccgagcccctcccagccaagcgggtggctctgcagaaaagctggctcgagccccgcccggccacacaaaggcgcggccccacccagcccgggcgcgagaccgcagaggtgacccccttcccagggattcagggagggctgtctcttctcgcccacccacggtccgcggagctcggggctttttttcccccagcccaagccccccgcccaccctctgttctctatgattttccagaatggagaccccgcgaggggcttctctaagggagaccctcgctcctccagcggggcgcggctcggccccacccctcccagctgaggcccagagccgcctaccgctggccgggtgggggcgcacgtggcgactgggtgtgtggagcgcagccagccctgcagagccccgcgccgcgccctgcgctcccctccccggagttgggcgctcgcccccgcggtgcagccggggagaccggtttctgcgcagtgtcctgagctacccccgctttccacaattcgcagttcactcgcacgtccagaaaggttctgagaatgggtggtgggggcgatctcgcctcgctttctgcacccctcagaaaggtttccgctgcaggctagtggctgcaaactcatcgtcatcatcagtattattatcatttcaaatcgttgttattatttaatgattcagtagccttgtttgttctcatttgttcaaaagggacgtggattgctcttggttaaggattaacccttgttgcgttcgctttgcttcctcctaattgccctcatccctttcccccacaaaaaggtaaatttgtctccagttgttcattttaagttataaagcaaatatatttttgcttcctgccaggattatgtatgttcatgtggctaagatacatgtgcaagtgcttgctaagagcagggtttgtgtgccaacgattgctggaaaattctctgcaaagaattgtttgtggctgcaatgggtgagaatacacatatataattgagatgatcttcaacataaggttatatctataaatatataaatatagtttatgcacaaaattttaagttttttcccctgaaactgttcttccaactgctgattcttgatacagcctcaatcctacacagatacatggatcgtgaaatggtagccgccatccaaataaaaatcccaccccaaatatgacaaacgcaagcatcctttctggccataatttaactgcatttgcaaatcatgaaaaaaacactacttctgcagtattaaaataatagattttgaaattaattccaatttcaaagataattaattatcagggcgagtgcttttttcctgattcattaaacaattatgtattcagcatgattgtaagaggtgcatataatattccccattatcttttctaatgaagtgggcaccttctgaatggatatataagtaactagaaatgaaaagctgaggatttggtcagaatttcaggataaaactgaaagaaatggcagtagtttatcaattaatctcatgtatttagtttataccaggtgagtaagctgagcctgcaataaacactctctgtcccagtgtaacacgtcgcaggtagctagaatgataggataaattaatagaccttgtggtgtttgtctatgcacgttaaaattctctgagagaaagtatattttaaaatgataattaagattggacatttgtgctattaaaatctacaactttagtcaaaattcacaatggtttttttttacaataatgtgacttacagatttgtagtaaattattctattctaaaagagaaatgagtgtttttattgttacagctattacctcattaatatttttagcaaacttttatttgttgcattgaaagcagttttaattactttgggtttttatttttcaaattactaatggatagatggtggaataagcatttaatcatttggcacaatatgacttccatcaaatagctcattctcagtgattaaaaaatgctacaagaggctacaatttactcagattcaggaaatgtcctttcagagtgccataaggctgattcatataataaaatagttttcttccctataatttaagatcaaatagttacttagttctgtgaatacctagcagtagctatcaaacagaattttaaagttaaatctgtacaactaacaatgaagtggaggatgaatcgatacatattgaatggaagactttgtcattgataaattcaggccatctttaggaaaattccggatttatcaatcaccattattttttacttcaactgagtgtgactgatcacatgctcaggctaccttggtagctcattgctcacaggaggctgaaaaaagctggcctccgagcaggaggaagctcagagcacaaacctaggcctgggcgtggccactgggagctgctgatagcgaaccccagctcacaccagtttcttttttggtcgtgggaagaaaaacacatattatcctgttgtcacaagatctgtgaccttatatgaaaaaatgctagaattttttcattaaaaaagaaaatactgaactagccagtgacccagatgttttcagaacctagactggttctgtccattggaaaacctcggtgtctgcattaacttttcaccacactagagggcaatcatgttctctaaaaaagcagatgattgatgtaaacctagttccaaatattaactgtttaataaaatcttttcttttaccaggaacattcaagtgtttattcaataagctgatgccatgctttaccctagtggatgaacagagcttgtacaattttcaaggagacaggatgaaatgagtggtcataatctgaaagtagatacacgccctggttaattattccctgatggttttacttctcagttttattacattgttattataataccatttatgttacttctgagattttgtagtggataaatagtagaaaaatgtcagtagtaatagcaaagttatttagcagccgaatattttaatgcttaaaaataaaggaataaattaaagaaaatcattgtttacttcttcatcgattgaaatgtgccccctgttcagagcacatctgaatatcagagtctccacctgcagagaacatgcagcttagcgagtaaaacaggcaggtatgtgatactgaggaggtgtaccaaaaactgactgctgttatttttcccatcttctaagtctgtctttcttttccatttaaagatacctttttaaatctaatccaatgtgatttcaatctagttttatcagatttcaacaattattgagcatctccttgtagtggttttctgtttattagaaaatcgatgttaattttaacgaagtaagaagaaatatataagtataaactaattttgggtatcatcaaaagtggattttttaaatatgcattgatagaattattttttgattacattttatgtaattctaatccagctataaaatatttaatagtgtcatattactgtgttcctcaaactttgatgtgcatatgaattacctttgattttcattaaaatgcaaattctgattcaatacatctggcttgaggcagacattctgtcttccgaacaagctcccagatgatgctgattctgaccactaaacacatcagttttagggatattaacttgtaatatacaggtatccctcctggtaagctctggtattatgtcttaacatttttaaatctatggtaatctttacaaaatattttacttccgaactcatatacctggggattttattactctgggaattatgtgttctgccccatcactctctcttaattggatttttaaaattatattcatattgcaggactcggcagaagaccttcgagagaaaggtagaaaataagaatttggctctctgtgtgagcatgtgtgcgtgtgtgcgagagagagagacagacagcctgcctaagaagaaatgaatgtgaatgcggcttgtggcacagttgacaaggatgataaatcaataatgcaagcttactatcatttatgaatagcaatactgaagaaattaaaacaaaagattgctgtctcaatatatcttatatttattatttaccaaattattctaagagtatttcttcctgaataccatgtgagaaaattcttaagaatttattgagtatgactgtatatttgaaaagagtgttttcttctgcttatctaagccaataaaggatcttcattattcaattctaactttctaaggaagtcaacctacagatcagaaagaggatcttcaaggaatagcatcaaagacatagtcaggtctcccatgcagtgactggctgaccatgcagccattaccacctttctggaaatattatgctgcaaaaatgatacaatacacgaaatatctcaaattaaaaaatataacatttcccaaatagggcactaaaaacatgatcccaaataaaactagcttcagggtttgcagaatatactgttactcaacacaaagttggactaagtctcaaagttagccattcagttgttgttaacagttcatttcagggtctctcagaagctgggaaactttccatttttgcaatttcttgtacattgaaggaaaggaagacacacttaagacagcattacaaaagtaattcatgttttaaatgtttaattctggcagtcgggcagggctctctgtataacctcatttggagatgacaaaaatctaaacttgagggcctcgagccaataagtcttcctatttctttactcaaacattttcccgcaatggtgctttctttcaactgtttttctggtgtattcataaattccagattctctatgggaagtaacttttattgattgatttaacccttgtatagcacatataacatgcaaggcattgttctaagaactttccacatattaactgtgttaatcacttaataatcctaagtaggttctattacagatatggaaactgaggcacagaaagttgaagtatcttactcaaggtcacacagttagtcagatccagaatttgggcccaggccatctggcttcggaatccatctttcaccgattgctgctagtctcatatctgttccatgttagaggtgagctcccattgcagaggtcacacctgtgatatcaccattttatttaaacagaccagagatggtcttctcctttctgatcacagactcaccttgaagagaaaatacttccaaattgatgcctagttttaatagcttacctggggcttattcaaataattgccatgatttaggctttgggagaaagagagctatgaggccgtgtgggttgtaacgtatgagacacatggcgttctgcaggctcagcacagcatcgatttctggtgggaacacactctgatgaccagttccagaaataacattgacttaatctcctcagtcccatcatggttagcacatttcaaaatgcctccttaactacttccataggccagagatatttagttttaacattttgttgaataaaataaatttacacattcacatttaatataactattagatgttatttcaagattctcttcatattaccatcaaagcaggcaggcaggcaggagagaactgtaggaaggttttgaatcccttgtgaaacatttttaattatcttttaataaaggaatcaggccctgtcatttgtcaaggagacatttgcagtagtaaagcttgtgtttataatatccatttttattagtcatgattaaagataacatttgtgtacatttgttctcacaaaacacttttatatgagtgtaaaggttaattaatgcatttcagccatcattttgctggtcatgtggaaatatagcttctttaggaattgtacttagagtaggagccacatattatactataaaaccataacaaaaatattttaagtttgttctcacttgttgttgacctccagagtaaaatatttaatactctggaaagttatgggtttcaaaatttattttatggcaagaaatagataattacagttctcatagagcacatttaaaataatttatttttatagggcaaaaatattgcctaggactgaatgatttttttttttttacaaagattgtaaagcaacgcctgcaagagtgcccatttagcagttattcttctggaataattgtattttggatgttggagttcgcacattaaccattagtacaagtacccaatataacaatagatcatcaggataataaatctgtccatcttttagttgtatgtctttatatcaggataaagagaattgagtgaaatttatctaaacctagtcccacaaatacttttacaagagagcatgttaaagtgtaaattaaatttttattagcattctactctgtctttggaagttttttttccttatgaaatgcagccataaagtttaacttccattaacaaagctgctcacagtaaacctattataataatagtttcccagtttgggcttcctagtgaggagcaacctaactcacacgaaacaaccccaacttataatatattgactgttacaaaactgagaccagaaaatcccatcaagatggtactgttatcatttccagactctcgggaagaacattaatcatctcaggcacttttaggatagacttattgcagcctccctgggaactctgcttcagaacataattatttttattaatgcagagttactttttatttccaacaaaaatatctattgttattatttaagtcttacagctttatctgagaaattccaattagcacccttctcataataaatattcaaacacatgaaaaattaccaaagttgttctagtcttttaatgacatattacatgatcctgcactcttgtcactttaaaaattatctttttattatatttctgatgatttttttcttatatagttttttaaaaggagcaggcaagcatagaagactaaaaaatgttcaaaagaaaaattaaatcgcatgatctatctatatgggaccttgtcatttttagaaaacattcacctgcttcatccttttgaatcttcatataatccctctgagatgggcatactatacaagttgtcttatttaaagattggtaaatttaagctcaaataatttattcagtggcaagcctcagaggcagactcggaacacaggtctaatatatattatatatatattataacatataatatatatattacatataataaagttgtgtatattatttacctatcaaaatatttatatgtaatatataaatatgttatatatcatgtatgtgcctatttcatacatatatacacattcatgcaaaataaggtttagcactccctccactgtcctgtaataaaacatgcacagtgagaatagtcatacacgaggcatatttgtcttcagtttaaagtcattgatagtcagtgtcactaactaaagtaaaatagattggagcaccaactttgttctgaagcctgtgccaggtattatgagaacaaaaataaaaatgttcctcacccttggtggatttagtcttttgcagaaaaaaagatcctgtacatgtcagaaagttcaatagtaataatggtaatttataactataaatggaagtcaccatctcacaatttcaccatcttaacaattttgttaaactgccctacaatattacaagatagtacataatgatacactagtaacatcaactaggaagtaccaagatccaccaaaaggctgaaaaatttaaatatttaatgagtccatcaaccaatctggccagagaattctttaattaaaatgcttcccaaattttactgagaatcagcagcgtttgaggagctagcctccacccccagaggttctcactctattaggtctgaagcaggtcccatggatttgcatttctaacaagctcccaggtggtgctgatgaggctgattcagaaccacacttggagtagacctaaaacagcagtgacctgtagggtccccaagcagcaggccaggacagcatgtgagttacgtcctctgtggagctctgcaacaaggcgtcaagaggtcagagtctaagtccccatcagctctgcccttctccaccagtgctgctggtgctgcatggaaggaagagcccagaagggattctgagtttcagtctttactcttgctgacgcaccttggtcaggtcaattttcctgtttgttcctctaattcagcatctgtaaaatagccatgtgaactgccttgtccatatcagagggtctttttcagactcaaggaaaaaaacgtgaaagtgattagtgtctgtcaagtagtatataaatgcaagaagttgagtttttaaattgtcattagatataaatacccatgtgcatgcatttagaatgagtaaagagggaacaaggagcgcaatcaaaaactgcgtcatttgctttttgaaaaatactttctatgtaatgaaaagtgaaataaaatgttaattgagtccctctgacaacagcatcagacgttttgcagttcttgtgattagaacccacctggccagcccttcttcctcctaaagaagagccttcttcttcttaaatgaaggttggctcagaagaagcaattaactcattcaacgttttgttacagtcaatccacatccaacttttccccaactcaatctgctttaagggaaggatggtaagtggtggcccaagatggcaaccatcaagcttagagaatctctagaagcaggggtgtccccagcaagtagacactgaaaatatgagagggctgataagccagagataaaactcagtacttactttgcttctagtccatgtctacccctttcttggcaccaccttgacactaccctctgagtccaccttcctgagatggtacaaactctgcttagacaaagcagcccatgtccaaaggtgttagggctcagtttaaagctgccttcaaaagttaaaacagaagtgtaaagttctgtgcaattaaaaataatcagcttgtcttggaactcaaacgaatgtaaaatcctatgaaaattaaaaagcagtaccacaagttaccccaaaagtccttaggtcagtaactgttcctgttacaggtaagagagagcatggattagaggtgggcgtgggtatccagtggacatggttttgaaccatgctccactactactcactatctgagaattcttaaatttattaatcatttctatattataattttctcagttatgaaatgggaaaacaatacctaaatcacatggttgttaagtaagcaattgattgttaagcatttggtcatcaaaaatattaatccccttccctgattccctagataaatgatgaaaatactaaataaaaataataaaaatttaaagtgaacatctcaattcttatactttgttaatttctacatgtattacaaatctactagaaattacttggaattgaggaaatgattactgcttaataattctttgtggtagagggagagttggtatcatatttatgagacagcagccaatatagtatatctcaaaggaaaaaatccattctacataatgccagaatttaatagttaagcattttatctaggtcacagcacaataagcaagatggataattaaaataaaagtatatttctcttgcatatatttctcatttcatgtttccctatcatattttatatcttaccttacttcaaatacatatataccttcaataaaactgagccttcttgcttacccaggaagtttcatcattcagtagaaataaaagatgactttagaaatattaaaatacaaaaatctacactgaggtcttttgaatgcaggaaaaagaattatatcacacacacacgtacacgcacgcatgcatacacacacacagaacctctcgttctttcttaacatcttatcaatccatcagtttcactcccactccgtatcacctgactgtgcacaatatctcattgccacctcccagtcttctccctgcctggcaccctcctgctctcctgcttccactttaaacacccttccttcagctaggtcttttctttcagggatcctcccgttgctttcttatctggatcaatttagccttcctcttctccacccattagtggataagcacgacaaagacactagagtcaaataatacaaacagaatataccttagatgagtatggtgatgaaaaggatatggatacttagagtttagcactattctctcagccactcaggaaagcaacgcctttacaatcaatagtgtttcaggtaccaatcaataatctgttattgctatttttaaaatctataaggtatcagtaaaatgtaattactagagcaacaaagatatcttgtgaaatcaaattagtattcatccagcaactgagtacaaaggtttaagggaggataactaccaataccaaaacattttaagcattttgttttgcctcctaaatatcaaatcatgtaaatgtgtggtacataaattaggaattatatttatgacatagctgcagacatattaagagaaatatgtgcttatatttacaagtatagtacagttctttttcatattagatactgttgatgataatctgcatataaaaatgctcaatattttttcacatttataagccataaaatacagctaataaaatgtgtttctactttctcataaacatggaatagtgacaaacaaggagctttatatgaaagcaccattacaatttaaactctcacaaggtcataatatattgcactaagcaggagagttcagcttatttaaaaaaaaaaataaactctaatgaggttctggaatgcagagccaaagcataaagatggaaataaaagaattgcatgtcttctgaactgacttggttgatgatttttttaaaaaaggttttgtgtcttctgacttggttgatgattttttaaaaaaacgttttgtggtagaacaaataaggtaaatgaaattcagtatttaggatgaaaagtttttctaatttcaggaacaacattgaagaaatattgaactaagcagctttgaaagaatcagattccatttgttgaaatttttctgagaatgaatttttttaagacagtgtacacagttgcagtgtgtattggttatggattgtggcaagctatattacaacttacccaagaaataaggaggctgggcgtggtggctcacacctgtaatcccagcactttgggtggccgaggcgggcggatcacgaggtcaggagatcgagaccatcctggctaacacggtgaaaccccgtctctactaaaagtacaaaaaattagccgggtgtggtggcgggtgcctgtagtcccagctactcgggaggctgaggcaggagaatggcgtgaatccgggagggggagtttgcagtgagccgagattgtaccactgcactccagcctgggcgacagagcgagactccgtctcaaaaaaaaaaaaaaaaaaaaaaaaagaaagaaagaaagaaggaaaaaagtcacttgaaaagaatactggactttgtgtccagcttgcatagctgaaaagaataaaaacctgtccacttaaactcattgcaaaaagaagatgtcactcctacaaatagcaaagagtcatgaaattattctatccagaaaagtatacatttcatccctttggataaattttagaagtgaactatgaatacatacggtgaggatagccagctaagaagtcaagaaggatttctcaaatttgctgctcagaaagatcatactctccacaaaacaaataatagcaggctttccaagtcaaccttgaatccagctttcctttatctttccttcttgtgaactttcactagtttactatctaacaatgaatttgacgatagccacataccatcttatagcaatatttgttatcatatcccttgttatttatcattcacctgctctgcttgagccagctacaagtcacatgtcccacgcactttttcctgtttgattttttacagcactttgagacatgtctcattattcctacttgacaggaaagaagccatggaaagttgagtgacttgctcctgatcacaaatgctggccaaggaagagtcgagtttcaaatctaatgatctttccactgcactctagattcctcattttgaactatttttttattttttgcactatagacttttttccacattttgaactgttttttattttttgcactatagacttttctcttatacccaactatattgatgacttcttttaggctagaaacttgtttcacttactttccctttcttcagattgctgcaatattggccaacatgtattgggtacttactgagtcaagtactgtgattgtgccaagtatcttataggaggattatcatcctcatttttacaggtgagaaaggaaaggaggtaaagtcacacacagccaacaaaaatggtagcaccaggatttgaaacaaatcagtctgacccaagttgactttgttaaccactgtatgcacagtcttcttagacatagtaagagctctaattgtgtttggtgatttgattattatgacaaagtaagtaagggaagcagggagaattataagaaataaggctccacaacacttggctatagcaaagccccttaaaacttcaaaaggtcacccaaagaataaagatcaggctgggagcagtggctcacgcctgtaatcccagcactttgggaggccgaggtgggtggatcacctgagttcaggagttcgagaccagcctggacaacatggtgaaaccctgtctctactaaaaatacaaaaattagctggatgtggtggttgccgcctgtaatcccagctacttgggaggctgaggcagggagaatcgcttgaacccaggaggtggaggttgcagtgagccgagatcatgccactgcactccagcctgggcaacaagagcaaaaaactctgactcaaaaaaataaataaatcaatcaataaaataaagatcaatttggagaaattaatgcttattaataagcaatgtcttgcacagcacttcagtttctcaatacattacctaactcaatccttacaacaacaccctatccccattttgtggataaataaactcatgttcagaaggttgaataaattatctaaggttaatagttcctgacctagagctcaaatcttcagtttctatcatattcttgcccttaccctggggtagctaacattcactcactagtattggagctaaaataagggagagaacatataaatgaatacaaaggagacattcacctgccttctctttctccttacatagagaaggttgattatctgctattgtgaagtttgccttttgaaggatagaaatgagaagactttcttaaattttgcctctacgccaagaaattagagtggtaccaccagtagttccattttcaaactatcactgtagctaaagctatgtggtaagggccaaggaaaagaagtattcttgcacttcaaaatgcactgaaataccagtcagtagcataatataaaggaatttagtggagagaagagttgacctcaatctggctccaacatctcggctcttaacccctaccctacacttgttcttcatggggaagctaattgggccactggaagattcagcagctaccatttgcagctgagggacagcccctccctgcttagcaaccaatggatatgcatttatggaacacctgctaactgcgacacacactcctatgtatgagggaaaatacaaaaaatgttaaaggagatgccttcccttgccctcaggaaacttaagtatagttgcaaagaaatgattagcagcaaacgaaaccatggagaagtaagggctaaggtctgtgaaacaagcctagaaaataaccttgtccttgaaaaacacaaaaagaaagaaagaaagaaaagaaactccaaggcccttgtgaaggaaaccattaagtttgcttcacttctgtgtttaggaagacacaaacccagtcttaatgaacctcaaggccacaactactggagacatttaggaattgtcaccacattctaatgtatatatcctctgtttggcccttcctattaatattttgtaaaatttttgaagatatgagcaatgtttaaaaccatgaatccccctttttttataagtaatatttaggctgaataaacaagagaaaataggacataaaggggagccaacgtgtgccttcatttataatgtattcccaagttgtgagtttggtttatcagcaatttatcatgccaaattccaagtcatatttatctatgcagatcaaacacttgattctatttttgccttaatttttttattgggtatgtttatgaccaagtcatatggtattttctgtgacagataaaatgcacaggttattccaatctggctcagccagtcatagcaacatgtagtccttctcatgtcttaagaatgagtatcaagaattcaaagggagttccagatggcatccaaaaagcttacagtttatgcatcacttattctaacagtagaaaaagaatatttgaagccaaaaatagaccttgcatgtagcatgtggaagagtagaaattgccctgatagttaaacaatttgaaattcaagacattaatttctttatgaagcatttgtcacatcataggtaatattttatgcctatcatatatatacttattatgaaatacaaagaaattattcattctatctaagactttgtatcctttaccaatatctctccattctcccacctccaccctagcccctggaaaccacccttctactctctgcttctatgagttcttttttagtgagatcatgcagtatttgtctttctgttcctgtcttatttcacttgacataatgtccttcaggcttatccatgttgtcacaaatgacagaatttccttcttaaggctgaatagtattccattgtgtgtatgtagcacattttctttattaattcatttgttgatggatactcatattgattccatatcttgggtcttgtgaataatgatgcagtgaacataggagtgcagatatctttttgacatactgattccactttgatgggatatatacccagtagtgggactgctggatcatctagtagttttatttttttttattttttattttttttattttgagacagagccttgctatgtcgcccaggctggagtacagtggtgccatctaggctcactgcaatctctgcctcctgggttcaagcaattttcctgcctcagcctcctgagtagctgggattacaggcacgcaccaccatgcccggctaatttttgtatgtttagtagagacggggtttcaccatgtctcgaactcctgtcttcaagtgatccgtccacctcagactcccaaagtgctgcgattacaggtgtgagccaccacgcctggcctagtagttctgtttttaattttttgaggagcctccatactgctttccataatggctctaggaatttacattccaccagcagtgcacaaggattgcttttctccacattctggctaaccagtctcctgtctttttgagaacagacatttcaacacgtgtgagataatatctcattgtggttttgatttgcatttccctgatgattagtgatcttgtgccttttttcatataactgctggacattaatatgccttcctttgagaactgtgtatacaggagaaaataatcacttctcagaggagctttcatttcaaaatatccgggaaaaaaatagaaaaaatggaaaatttatcctagagtaagttgtcttttatattttgaccctgtttgtgacataaactggatgatacaaaactggaatgcaaaggctttaggaggattacttacttacttgtatattgctttaggttgtttgcagaaaattatactaattgaagttcaggctatgatgtgataaaatctatgtcaggagatgagtctacatgcaaagtttgaggaagtgacatttgagtttcaaaacaaaaaagcaattttcaatgtcatatctaggttaacccaaaagatttctttcaccctatttagctgcctctaagatggatgctgaggataattacactgtagaacaataggacgatgcttcacactcacctcacaggctctgttattcccacatactgccagagatactccaaaataaaatcactgcaacatcaggcagttataaacctcaacggtattattttctatttatatacagtatattttatattttacaagtataaaatagaatatatttattctattctctttgacacaaagtgaccataagacatattacttaagtatgactagcaaagtcatggggcttgtcattcaggaggaaactcttaactaactgttcagtttttgttcactgcaccatttacataagccaaactaatgcttcacactgtgcaaaacaatgcacagtgttgtgaatgaatggctaaaataaaactctaatgagtggggtttgaaaaatgcaactttagaaaactgttgagaaaatgttgcacactgcgcattttacaaaatttcgttgaaggacactggatattctttttaggattatggagggaagcaaaattttggctcctacatgcagtttttgtggcctttgcctgaaatagtcatctcccattaattatttagatatcattcatttcctaagacaacatttagggagactgccttaagtacaatttgtacactacccagataagaattctttttggtgaaacatcgataaatattacttggcagtaacaccaagttaaaatatttgtttcacagtcgacgttaataactattatagataaagtgaattttataagacatactcagatctaaaacagcaatatggagctcttcaaatccattgaaacttcataccagcctacggaagtagaggtttttatgcaaactcttcaagaaatatgctctgaacttttaattccttagattgatagaggaattaaatcatgatataactaataggtttgtggtacaaattgctgctgcttaatctgactctgtgtcttcccagtgttctatatgaattagatattccattatctaaagacaatcaaccccatcccacggtgatagctctaggactccctttgagttcattaaatctgtattctcagtctccaaacttctggttaattcaaacagaaaagtcaactggcccatgaactaaaataaagtcatctgaattttttttttattttgcagtgtgataaaagtctcgcactttttatttctgaaagtttctgctttcactgagagcataataggctatccacccttatgcaatcttacatacaaagtcatagtcaggctaaattcaaaaacacatgtgagatagaagtcaacgtttattttctggagaaaagccacacattacaacaaagtgaacaatgaagctggcatccttatcactggtgaccaaaacatttgtgactctggacattggccccacaaatgcgataaacattctgcataggaagtgagttttgctaattaaaaatggatccaaaatactttctactcttcagccaagaattaaaaagtaatagggaggaattgaaatcacttgggtgctacattgagccattctggagaagcaattcagagaatgtcatggcagcctcaaattgctgctcaggagcatcccagcttagaagattgcaggaaaggaagagcaaagtcattcttacatgagaactgtccttaaccagatgaatagactctccattttttaccctggctttgtctcatttaagtcccaaccaatctagctatcattttaggttttactacctgctagtatttaggagcttagggggataaaaaaatccctcaatactcagaattagacttggtgataaaaatcttgacacataaacagaataaagcgctttcattactcctctaaaccacagtgtcatttggtctctatcaaggactgtaagaatttctttcatcaggggaaagaaaaaaaggacaagagcctgcaagatgtagcggaactctcattaaacacagcaggagctttaactggaatccagagtaaggtgaggtaccaggttacaacaatttactgcttttattacaattttgatcacaaggactgattcatgtcatctagtttcttttccttgtcactatcactggtgctaagaatacatcaaattgaaatttaagagcctcatatgtttctgtataacccagtgatgggttgtactgctttgaccttcttaaatgtccctttatttcatttgatatccattcccatagaaaaactataatgctttggttggtcaaaatattaatctttcaaaacctccctggcttagaaaaccaaatttttgtagagagagatgggtagaatctaattttattctaaagcaattagcattacatcatcacagcagaaatatctagaatattacctcatgtcagtgatcttctgatatgttaaaaagggtattttaaaatctgagttatttctttttctttttaaagttacatcattaattacatactcatcaaccaaaatattttatgctccaaatttgaaccgatatagtatgtaagaagtgttcaaaatgaaattattttggtctattttgtctttgaagaagatcacagggatggacctcccaaaaggatttttaaatgggattacatatctgacttttaaaaaaaattatctgaccttgagttatagtgccccaaagtaagcaaagttccaaacacacagtatcatcagaattgagttaaaattatcaccaggggcttaatttctgaaattaaaaaggaaatgttatttccttatgaaaagaaaaggaaccaaaaatgaacttcaaggtagctgatttctgtctatgttaagacttaggtaatgggagaaagggaaaaggaaggacagaattaggagaggagcagtgtttaacaattgcgggtgcaagactcaagttttttagaatccattagcagagaaccctatttctcccattaactgctgtccttttaaatcctggcaccagctctgaggactgcagggtccatagctagtgccccactctacccagtttaaagacaccactgcctggaaatgacaggggtttttttcttaaggaaagaggtgctttctgccacgtatatataaattggtaagcttcaaataaagtgcttttgtcctttctgtctatcagaaactgtgcaaatcgaattgctgtaaaaccaagggcaagagacatcaatcctgcattctatagcatctgattttatcctttatccccaggcacatttcaaaaggaaaaaaatgaggttgcatttaaattgagtatttgggacttgccaggaaaacctcccgctagactaatatgattgcagggaaaacaagagaaaggaaaagtggagagggagtgtgctaacagatcctgggcctcgtcagcagagccgtcctgagcacaaggccatggtcagacatctggtcccgcgaatgacgttttctttatggtcattaagaacaccagtgtgtcgggacacaaacaagtattcctttcagggattatgacacattttctcccaaagtagtatattaatgacatttccagagcattctttactatcttttatatgtgatcaggaagactaatacatatcactacttcttttacacacagcattagccaaaactaaagtgtcaaatacaattttgcctaggatgaataaacagaagaaatttttatgatactgcactatcaattccaaattaaataacaacaaaatgataagtgttaaaattcatattaatgattgttcccacacaagccggaaaaaatctttctaagaagtctttcatgagttaatcccatctttcaaagtgttcagtggctccgaattcagttactgtttcctatcagttcttctttcattaagtctcttcccttttttttctctttgcactatttcccttagccgggtacataatctgctgtgctttattcatttgtgtcttaagtttgtttcccgatgacatacctttccagcaacgccatctggggagtttgggcaactgtaccacgttaggaggaaacccttcttcacaggagagtgtgcctttgctgcagggaaggaattaggatttgcttggactgtggttgcagctggcttttaaggatctccttagaatgcaagcaactcatcaatgagaatctctgcaatggttgtcactgggtagagtcatgctatgtggggtcatagcctttgaaacaaataacagtaaagataaaaatgctattaaaggaatcaccacccacagaggttaactgggttttgtccccagaccacctcgaacaagaaagaacatttttatcagtcattttcttagttttagctgataaaacaaagtaccatagactaggtggcttataaacaacagaaatttatttttcacagctttggaaactggaagtctgagatcaggccgccagaatgatcagattctagttagggcctactttgcttttgcagactgccaacttctagctgcattttcatgtggcaaaaggagattgagctagctctctggtctcttcttataaggacactaatcccattcatgaaggcttcaccttcatcatctaattactctccaaagaccccacctccaaatactatcacattgggaattagatttcaaatacaaattttgcggggacacaaatattcagtccataatagtaatgattactcattatacatagggctctaaatgtgctagcttctgatagtttttacactcacttctctttattagcttgtcaagcataattagggcagtggccttactgaaaattattgaatttagtttcctaaggacagatattgaggagttttttcttcactaaaaattcacgttccgatacagctttcatctgttactactttgtgagatggaaaatcttttattttatttttatgtttggattgacccttcttaataaagtcggcatgtaatatgcttcatgtgtttctaatatgtgcttaattttgcaaaatgttttgcataccagaatgcatttctcttccaaaaaaggtaccagcctacaaaaccttgctgttactgttttcaattagttcatggaattaaatgtattaaatgttttatgctctggcagaaattatgattctcacttaactccatataaatctggatctgcctgggcctttataagtgacacaatttcattaactgaataaacaaatgatacaaagaaatttggtttagccttctaaaattccaaaggcgttcaacaaaatatctcagaatggatgttccaggacttttatggcacaggacaacatgtattgcttattttaagaaaataagctaaatagtgaggggattcttttagcagatcctcaggatgtgttaggttgaatcataggcaaatgatatttgatcattgcacctgttaacacattgaacctcatcctaaaattgtagagctagaagaaagccttctggcagtttttaaatagattgatttactgcaatttatccagaagcttcaccgttgtcactggctacatgtgactttggcctctgtggggctatatcctcatttgtaaaattggtggtgaggtaggtggacagttgactaaataatctcttagaataattctagtatctgtggatctaaagcatccaggggttgaatatgtttctttctggccaagaaaagatgcacctgtcaataatgcccaaactcatcttctgagaatcctctttcccaagatacccactctcccttgggttatattatagtaatgatcagaagcccctgccaagaagaaactgttaacctgggaggtctatattttatttcacagccatctgtttatactttctcacaagttagtgcacagtatacccatcattttctaccattttccttaatttattaattttactaattgcataattaacaaaagtaagaagattttacctccttatccccatctggtagtttgcagatacttggcctgatgacaactgacagtgatgagatactcaccaagtttaccagggcaggaggcttcctagagaaaaaatgagaaaatgaaatggggaaggggagtgaaggattgaggaggtgacaatctggactcttgcaactgcatggcaaggttggcacacaagctgggttgcaacggagggaaggagatccttatcagatgtaatcagagctcagatcgagggctttggtgtgtgtagaaagagggagagacaaagaacttaaaacagagctgccatttgaccttgcaatcccattacttggtgtatacccaaaggagaataaatcattctattaaaaagacacatgtgcttgtatgttcatggcagcactattcacaatagctaagacatggaatcaaactaggtgtccatctatggcagattggataaagaaaatggggtaaatataaagcatgcaatacaacatggccataagaaaaaatgaaatcatgtcctttgctgcaacatggatgcagttgggacccataatcctaagtgaattaacacaggaacagaaaaccaaatacagcatgttctcacttataagtgggagctaaacactgagcacacatggacataaatatgagaacaataaacactgtggactactagaggggggaaggagagaggtttgtaaaactacctatcaggtgctatgctcaatacctgggtgatgggatttacaccccaaacatcagcatcatttaatattcccatgtaaaaagactgcacatataccccttgtatctaaaataaaacttgaaattaaaaaaaaaagaaagaaagaaagaggctggaaatagaggctcacacctgtaatcccagcactttgggtggccaaggtgggtggattgcttgagcccgggaattcaagaccagcctgagaaacctggtgaaactctgtctgtacaaaaaatacaaaaattatccaggcatggtggagcgcacctgtagtcccagctaatggggaggctgaggggggaacatcacttgagcccaggaggtggaggttgcagtgagctgggatcacaccactgcactacagcctgggtaacagagcaactctgtctcaaagagagagaggaaagaaaaaagaaaagatggacagataagaaaatgcacttggagattaagagaaagcagcaacataggaccctggataatgtgtttgcttaataactatcctgatgagttatctgactattcccaaatgagtacgtggcaattcaggctgaaccatcagagtagccctccggaatcttacttatgtacaatagacctgcatgcacatttactagaatgagcctctctctctggtaatcatgtctgcttccactaattccatctgtttcctctctctccctcctatcctgctagatcttaattccttcgaccttcctttgtttttctaactccctttctttctcttgttatttaacctgctatactatgcaattgatctcctctgcactaaggaacatgcacttcagaattctgttgacatcttgcattcctttatatttagtgaaagaatgcaaaggagtctacctggcaatattcactctgcaggaggcaataattattattcaaattaaaggaagcagtaaagagaaattcagaaaaaatgaaatatactaatcttcagcttttcatttcagcctacaaggaaaaaatgaaggagctgtccatgctgtcactgatctgctcttgcttttacccggaacctcgcaacatcaacatctatacttacgatggtgagtaacctaggatagacatacccctgctagctagatcatttggaaaggttgacatatatttgtttcttacagctcctgatataattacatcaatattttgtagctctcactattgacttgccgtgtctagctattatgtccaattgattacctattgctgaaaacagtttgaatttggtgctaataacaacacatcaatgtctgttaagaaatgtggatggattcttattaacagccacatccagcatatcaacatccacaatatgtctaaggtctttctttgcaaataatttaataggctaagccataattggagtagatcataatttgtaagaaaatgctttatacttagaaaactcaagagaaagaatcaacaaccataattgtttttgctttattgtagtctttataaagtttctatactttgtatatacatgtcaaccagctaatgataataataattggctcaataaataaaactgacttacgactgaggccctagataaagagggtctgaaaagaaaagcctaaagaattagcatggcaattaacatgattgaggtgcaactctttaggtttgatttatcctgattcattttgcttactttggctctgccacaatccacatgatcttggtcaaatagatacttggattctctaagtctcatttaactctagcatcttcctcttggagttgttgtgaggtttaaacggtttaatgtaagtcaaatatgcaaaaccaagcctagctcattatatcactctacaatgatagctatcattatcaacatcatccttacctaattcagtcaatttaactaaaatattttatacagttctatgtatcctagatatccctaaggcatattttactaactctcaggctcacaaatatttttcttttccatatatgtaaagaaagacattaatgacaaaacaaactgaccttgtggcagttaaccccttctgcacctttaaagcctattcaaggactcaaaggcatttaccttccaaagttattctatcgtagcacaaaaatcataaatgctaattaactgttccataaggaaatgtcctccatgtgaaaggaattctgtctccaaacaaaacattcattagaatgcagggccaatgcctactttgtacaaattcattcggtcagcaaataaattagacagacctttattatttgctagatgtagctgtgaagaaggatccagctatgtttcttatgagactaatgtcgaactatgggttgtcactgaggatccagagttccatagggcgtagtcctcaccttcaaagaattcagggcttagtagaagagtcttacacaaatgactagaatgtagaacacagagtggttaggacaaaggagccagggatggtttttgctgggttagggaatgaaaaaaggggaagaaaatatgtgaagttatgtgtgagctgattcttgaaataagctgtttttatttgcctgcgttctcttataatccttttccataggcttccataatttttattgagctgtatttaaagttgaatagataattcaacatttctcgtaaactgtgcttcctaaaagagtccgtagagaatttcaaatttctgcagtctttaacttgacctggtatttctatgttagataataacgtgacttgtttattgcaggcaaacattataacaataaattattattattgtttacatttgtaagcactaagtatatggcttgtgctttgcattcagcatcctttatcatttaatcttcacaaccaccttagaaggaaggtactctttttatttccatcttttaaatgaggaaataaaagcataaagaagttaattaacttacctagtgtcacacagctattaagaggggcttactatttggatgcaaatataggcagttctaattccagagcctctaatctaaggcatttaaaaccccatcaccttatcaaataagctgtttttatttgcccgtgttctcttataatccttatccataggtttccataatttttataaaattgtatttaaaatttaagtataatcttggatgccatcaggaaaatgaaaaacatttttacatttgtgaaggaaaaagcccacatcatttccaatatagttattgagttagtattatctagactatctattagcagctaaggatctgaggtcaaggcctgccagcctggcattttacttgaccacaacctccatgtgcactaaccaggctgctaaaagaacattaacgggaacataacctgctggcttggttgccacaattttaaaaagacgttaataaattagagagcacttagaggttaggaaataatatggtggtaaagatctagaaacagtgtcattctggggcacttgaagatgtttagcctgggggaacaacttgaaatggaacataactgttttcaaatacttgaaaaatggtggtgcaccacagagaatggcctaatcatgggtagcttcagacttcaaacaaggatcagtgggctaaaaccagagagatggagtttgggactcaaagaatgctcatctgaaattgagggctgaccagcgaggttcttttaaaaatcattgcattttactaaattgtgagttctgtaattataaatgtcctagcaggtgctagctgtcatcttttctattataaattatactattttatgttataatttgtattatacaggcttaaaacataagggtctgataatctgcttatctttaatacataagccactgatagaaaataagtggctaaccattcttcagttctttttttaattgacaaaaattgtatatgtttgcggtgtatggcatattttgaaatatgtatacattagagaatggctaagtgaagcaaattcacatatgcattacctcacacacctgtcatttatttgtgatgagaacaaaaaatctactctttcagtgattttcaagaatacagtacattgttattaacaatagtcagcatggtgtacaataagtcttctgcggccgggcgtggtggctcacgcctataatcccagcactttgggaggccaaggctggcagatcacgaggtcaggagttcgagaccagcctgaccaacatgctgaaaccttgcctctactaaaaatagaaaaattagctgagtgtggtggtaagcgcctgtagtcccagctactcaggaggctgaggcaggagaattgcttgaacctgggaggcggaggttgcagtgagtcgagatagtgccactgcactccagcctggcaaaagagggaaactccgtctcaataataagtctcttgcatttgttcttcctgtttaactgaaattatgtattctttgatcaacatctccccagtctccacccctaacccctggtaaccacaattctactctgcttccgtgagttcaactttatgaatagtccacatgtaagtgagatcatgtggtatttgtctttctgtgcctagcttatttcacttagcatagtgtcctccaggttcacccatgttgtcaaaaatgacaggatttcccccaacttttttaaggctgaacagtattccatgtgtatgtgtataaattagattagtagatgttgccactccctcctccaccacagtggctctatccctggctcctggctccagccgagtacactagaggaggatattctaaacagcaacaacacaggagcaaagacattacaatggggtgttgtcttattgcccccattagactgtaagcatcttgaagacaaggacccccatcacagagtgatgttgtcatccctggagtgggcactgtgcatgattgatgactggaagcaatgaacatacagaagggcaaaacagaaatcagcaggatgctttgcatttcagcattgactttgccaaatatgcccaactgttcagggagttacattggttctaacgaagctcctgtgattcctaagcacaggaatggtgataatatatataatggtgcatgcatatatacgcatatctagataatgatatctcattatatgtgagaactgaagaactccgttatgtttctcgtctaaccaaaaagggcctacagctacgataatttccaaacaaataaatctgtgctacttgattttcatgcaaagctcatatttgttcaaaaggaaaataaagcttaatttaaaatcaatttaggctatttttatctaagtatgcttaccgttattcaactccctgcagatattgtcaaatttctcaatatggtaaatatttattctgttaaaatatatccatagttacactaaagacagagaggtcttatatgttctaaacaacatagagcaaatgctcataaacagcattttattcctatctcccggaataacaacgctacttccaattgctggaatctaaattattaaaataaacccatgctgcaagctttgtatgcttaacattctcaaatgttcacttttcagatatggaagtgaagcaaatcaacaaacgtgcctctggccaggcttttgagctgatcttgaagccaccatctcctatctcagaagccccacgaactttagcttctccaaagaagaaagacctgtccctggaggagatccagaagaaactggaggctgcagaggaaagaagaaaggtaactttttccataggttttccttctctctctccctcccctgctcctccctctcacacactcgggcacacatgcacgcacacacacacacacacacacacacacacacacacacacacacatacagagagcaatgacagctgaacctgtgccatgccaacatgtataggttttcagtagacacagagccaggctagttggggtaaaaactgtaagatagatgctaattttaggctagccaaaccagagctctcagaaatccaaagagcttcagtgctctagtgccccttcccgtatattgaatccccttattataaaagcctcccttccctagaccatcaggcagaagcactgtagagaaaacacagccctggcgaactccagtggtggggaggggaagaagtgctgcttcctccctctcaggatctgtgtcaccccctttgtcaggcgtggttttccttggaattacaaattaccagatcttccctccaagatctttcctgcccagggtaagggccaagagcttgcccctttcctcttcagagtcccactgcctgccctggaagttggtccttccaagatcaggaccttctctgagttctttgaatatgttctttatctttttctaagacttgatggggatttttctctttttgccattggtccctgcttatattaaagagctttccttttgccaaatctttacttttccataatcacatggctaagaagagccaagggtattatttgagaacacttagaaatcctagggactgtgtacacaaacagaagttgtttgaatgtgtctgttccaaccatgtggttatggtagttaatcccatcaaggtactcacgatcatccaaaaatggaattcttttatgtaattcatccccacattgtatttcccaatattttttatgatataattttagaatcaggtaatcactaagaacatgttccctgcacagttttatgatgttttctctaaaaagtcagccaaaactttggacacttctatgttggataattaaaaacagaatgaagataatcctcctcctaaagattgaattctccaagagagaatgcaggacaaacacagatgtgctgtgtatagtatatgtgcatatatacatgcatatatgtacacaaatatgtgtattatcaaataatgaggctcaaacattagaaatccttagattaaattttctaaacaagaaaacactaatctttgtagttgaaaaaaaatcctcctatgatatgtaatatgctgatctcaattttcacctaagagtgatgttctccaaatgtccgatgagcatgtcatatatatatatatgaatttttatatatataattacaatggtaattggtatatagagatatctatattatagatatatatagctatctctatatattacatataccaattatagatataaatataacaatggtaactggtgtatatgtgatgtgtatatatgtatatgtataccataattatatattaatattgtatatatgccataattatatattaatattggtatatatacaccatgattatatattaatattggtgtgtgtatgtgtgtgtgtatatatatatatatatataaaatactagttatcattgttctagatttaaaaaacaggaacctgagctactaactcgactatatatatatatatatatacaggaagttgctttaaaacatttttatcagcttttttattgttatttttagctttattctcatagtaaagctaaaataaattattcaacattatcaaaactttgctgccagcagatgtaagcaatacctaaaacagtggagagcatgttgcacccaaagcagtttaagctctgacccaagcactggcatcttataggcactgggtagagataagagtcataggtcgacatatattgagatgctatgacttgattagaatatggagtcagtgactgaggtgaaattaaaactcaaaccacaattcaacatcctgatttaggatgttgctggtgtttctaggtactacacttaatttgaaagaaattattgaggataaaaaaagaactgggatcaacaaaattaactaggtgttcttataagagtccctgaggttactaattaatgaaactgataaagctcctgcaccctgacagcaagaaattatcaatgattatacatttaaacaattgaattgaactagaaactggccacatggttaaaagacatttacaaatgtaatcatccagtgttatgatgcccagaaaaaaaaaattccttagaatgctttaaaagccgtattccatcacctttccagttatttgttaaacattttgtaatgcaaaaataaccatatagattatgccctagtggtcgggttttatttttagttttttatggtttttttttgttaatggtagagttttaattaaaagaaaatacaactaattagcagaaagtgccaactttaaaaaatcactaattgattttattctattgggttatactgacttaattagcactaatttaaagaactattaattatctttaaagagtctttagcaagtgcatatatctcagtaattatgttagtaaggacatgcctataaccaaaacccaactcaactagttaaaacaaaaagcaaatatgtgactaaaaagtctaggagtggctacagcatcaggaacagctggatccagggatcacagtattatcagaaaactttctttcagtgcctgtcatctcttcctgcatttaactggtttcattatcaagaaagtttaatttcaatagtcagttccaaattatttttctcacaacttagcaactccagcagaaacagagcttctttttcccaatagtttaacaaaagtcccgaaattgagtctcaatggcctggcctggatcacaggcccaacccagaaccaatcattatggccaagaggatgtagtagtttgatatgctagcctgaatcacatgcccaccactgacctgcaaaggattttaggtaagatccctggggtaagaattgtggaggggtagttccccagaagaaaatcgaggtgttctcacaagaggaaggggtaatggatcttaaataaacaaaactatagatgtccacattttctatctataaatgtttagtgttactataacaattagaataattatttagttcatacactattcaatttgtatctcccttctgttgccctgttgccgttattttcttacagatagaatgaaaaatattaatctaggcagctctgtgaaacagtactgtccaaggaatataacgtgagccaggccgggtgtggtggctcatggctataatcccagcactttgggacgccgaggcaggtggatcacctgagatcaggagttcaagaccagcctggccaacatggcaaaaccccatctctactaaaaatacaaaaattcgcagggcatagtggcgagtgcctgtaatcccagctactggggaggctgaggcagaagaattgcttgaacccaggaggtggaggttgcagtgaaccaagatggtaccattgcactccagcctggatgacagagcaagactccatctcaaaaaaaaaaaagaaagaaatgtaatgggagccatatgtgtatttttaaatgttctagaagccacattttttaaaataaaagaaatatgaaatgaattttagtaaaatattcttcacccaatatattcaaaacattatttcaatatgcatgtaatcaatatagaagtattaatgagctgtttcacattattttattcatactaagtgtttgaaatccagtgtgtattttacgtttacaactcatttcaattcatgttagacatattcctagtgcctagtagccaaaggcagccagtagcacagatacggatattaaaacagaaaacacctagtgaataatggggaaattttaggcctaagtttttaaaatccataccagataattattcagattcaaatttactttgttttttcatatatattctttaaaaattacattaatatgggaactcagaaagttcaaaagaaatttccattctatggttttagtctttacattgtcagaactaatgcaagtgtgaagtttaggatgtactgtaagtaataggatcttctaaatctcatgccttcttcagctacctactctgtttctatttcagttcctcactgtggggaggggacttctctgaacctaggtttcatctctcactctcgttcatggtaaacaggttttcctttgtggcacctagcacaattagtaagtaattagtatttactggcatattagtatatatatgcatatgtatttatttaaccctatgtcttctactagattataaactccatgaagatagaacttgtcttttgtttaatagtgcttggcaatagttattactgtaaacattttttttctttcttattcaactcctgttagtcattgcctgagtactacaaatgtttttaagtaaattaataaataataactttcagggccaaatgtgaaagcggcaatatatagcttgttttgattttttattccaccctcccatcctaaaacaattatagtcactaagtttccaaatgacatctgaaattgcactaaggaaatcctagtctgggcaaaatcactcagtcaacagatatttatcaagcacttactatttggcaggccctgttctagacacaggggatactcatcaaacttacattccagtgggggagaaagagctaataaatacatacacagcatattagatgatgcaaaattagcaggacaaagagaactgggggtgtgggggtgaaagaagctaatattatatgttattattactatatataataatataattattggatagtcaaaaaaaaacctcttgaataagacatttgaaaagaagcacaaaggtagcaagggagtagggcgggcagctcttctctgggacctgaacattcaaaatgatgagagcagcaggtgcggaggccctgaaataggaatgtatgaggtgtgtttgagaaataacatggaggccagcgtggctgaagctgagagcagggggagagtggtagcaactgaagtcagaggtcacaattaaggactttgacttcacatgaaatgggagatcatgaaggataataaagccatttcactactttatgtgaatcacagcatctttttaaagaagtatccttttttaaagggggagatgactagaaaaataaatagtgttagataaatagagaaaacaggaaaacattctagactaagacagtgattccagaactaaggatccacagaggcgagaatgcagaaagtgtaggtttcagagcagtgggtagactaagggtttggactagtggatttggatagggagttggagagtagcgaggtgggattagggagggctgtgaatgccaggttagtgtgcaaactccattatataagcagtaaggagtcactacagacttttcaaaaatacatacatgttccacctggcccacgggttagcaacattttcgttgccctggacccatttccttcccaataagttacaggtttgtgaagattctacctagcaaacatattacttttaaataactattaataaattatcttaccatgattataatcaaaggaatctgtaattgctaattatttctgattattaaaagataagcagtattgcactaaattgacataattctaactcaaagtaaatatacagatagacatggctatagatgtgaaatatgatttctgttagggctttttaaatttaaaaaaacttacgagttctcctccctccccctacccttaataccttgaaggcctctttgtgggacttcagggaccccttcagggaactatgacctaggctgtatttggggggctttctgggtttatagctggaaggctgccacagaggcatcgccacttgggctcagattcactttgtgttcaatgttttggcaatgtccccacctccccattccatctgttgacactattgcagcactgaccatctggttactaggttggaggatactccctcgggctcctttgaaccagaattagtgctccagtgattagataatagaagaagcttgtcataaaaagaataagccctttccctgctttttctccattctttgattatcgctggtagtcagtgatgatcatctctatgagtctatatcaatctcatcaggtcagtttgaacctcatctcttgaaatcaaagtttccataatgcaactgacccacaagggtgaaatgacatgaatgctttaaccatccatttatcatttattcattcattcaaccaacatgtatttagcaagaggcagcagagttagcataactatacatcccagttggcccaggacaactccagctaactctcgttgttttgataccattattaattatttctctttactctcataagtgttccactttggacaatcaattacatgagcatccttagcagggcacagtgtttaagggcatctttaaaatattgtctttaagaacatgtggttaagagaatgtctgtgttcaaatcctggttccaccacttaaaagctgtgtgacctcaagcaagtgacttaatctccgtatgtcctcctttgtcaatctgtaaaatgagactagtaatagaacttatggagttagtgtgagaattggaaggttactctacaataaagacatataaccagcatggtaaaagggttagcaattactatgtgaagaagcatccagtttctgacctcacagagattatctagcaaactcatgattttataaagaaaagaagtttctcatcaacagagactgaaatgctaccatacaatatacgttgctttttttttttttttttttttgagacggagtctcgctctgccactcaggctcaggctggagtgcagtgttgccaccttggctaattgcaacctccacctcccaggttcaagcaattctcctgcctcagtctcccaagtagctgggattataggcacccaccaccacacccagctaatttttatatttttagtagagacaaggttttgtcatgttggccagactggtctcaaactcctgacctcaggtgatccacccacctcagccttccgaagtgctggcattacaggcatgagccaccatgcccggccaatatttttaaatattataaaatattctttatcaaattgcatagaagaaaagacagtttgataggtaatagatatataaataggtcaggccaactaaaagtgtcctgaaaaaattaatattgtgaaaacaaaaggattttaatgacattgataaaatctcaccctaaaagagattaaattaaaaatcaccctacttgaaccagttcagtgagatttcattagcatgctctcattactggcataatcagcttcaaagtcactaagcctctgaaaggaagatgtgttgcttattcttaataaaatggcataaaagtagatcattagtcaccaaacatgatagacttaccttttccatttgttggcatctcacattgtagatggcaattaaaatggaatccagggaaagagggggtggtttgtatagcaatggattatgaaacaaagtactggattattcaccgcttgacattcaggaaacattctgctccttacagaatatggcacgtgggccacagaatcttccgtgtgctaccttctcggtgaagaagagcacccccaagtttcttttcctaggagctaaccacagtaaacccattacacactttagcagaagggctcattctaaaggtcttaggattttaatcattttaaatttcctgttatgcttcaggctcttcaacacaaagtgaatattgtactctttggttttacataattatattcaattgtcatatttcaacaggacattatttgtgactttagatgggtcaataatgattttcattgtcagcagtaaagtcaataattacagacacatcacctaccctacttgtgtaaaagcattttttggtactaggagatttagtgtctgatcaacggtcctggatagcaagtaatatatcccccaaataatgaaaagtgacaagaaaataaatatgtttacttcagaaataaatggaaaattagtgctatctaaaatgtagtcttaagtctcatctgtgtacataaagtaaaatgagttttatgtactagttactcaaatttatcttccactccatttgtatagtaattaaactcttacactcagtaatatacaaattggtaattaacctctttgcaaaatgttaaagtgttcctaaatgtacaataagtctcctttcctgtctcattgtttttcgcttcacgtacctctcatgtaattatttcaatgattgagttcagtgtgaggaggtttatgcctagaaaaggtgctcaccaataacgtgcctcagttcccataatagcaagatcgagaaggttctttagtctcccggaacgtcacgttgaacatctcagttctatattttgccttgacatttgcattatatcagctgatcattgtcttgccctaattttcccttttaatattttagtgaccttctatgttaggtacaggttatttagaagtgttcctccaaggccagatactttttccttgaacaatttatttttaacaacttttagcgattttctcacttcaccaccctccgtttcataagtccacgcaatcacaattcctttctgctaatctgcacagtcaagatataaagtaagaatacctatttgaacatgtagtgagaactttacttctctgccaaaaatgaaggaaaatgctgccacttttgtatgtcacatgttttttattctacagcctcactcacttcatgtcatgttttagtgcagttttctggactaactgcttattttctcattgattaaactgcctatttgctcattggaattagagccaatttttttccttgagggtctgactagaagattaaactatgttcatgtgagaatcaatttctacctaagaaatgagttagaggagttatgggcagcaatatctatctggatgctacactgtgaaaaaggaagcgaggttatgcctttctaccccaatggggtagcagagacctcaggaactgaggtagatgcccccctggttattagcgcccctgaataatttgttcaaaaattgactgctggacaggtgtcgtgttgcacgcctgtagtcccagctgtgcaggaggctgaggcaagaggatctcttgagcccaggaatttgaggctatagtaaactaaggtcacaccactatactccagcctgagcaacaaagcaagaccctgtctctaaatttaaaaaaaaatattgaatgcttatgaatagagactaatataggaagtcataagtatttccttgggatagaatgctttccaccataattgacttgacatcctgtatttttgtatgtgtggacttaagttttaaatatttgaaacacagacaattattaagtcctgcaaatgtgtgagttaatagtggatataacattcccttccagggtgtaagaaaaggtaccacagaagtgagcagccctgaagcacagcctggcctagtttggcaggtctctgtgagttagcagcagactcacgtgaccacactctgtactgccttctgtttctgtttcaccccattaattgtgctaaagaaatgcacttgacacctatgctgtgtaatctcatttagccccaatagcaacaaaagtactaaccccattaaattgagtcatttcaaactgagccaaatgttgcactccagtaaatggagtaggcattggttataatgggaattctccattattcataatggaaaccacaggagtttgttcatgcagatcaaatgtgtcccaccaaggcaagaagtatggaaaagtggtgttgctgtattaccttgtaatttcaaagccttcccgtctgaatcttatttccctgctgtttcctcttgactttggttctttcacaaaggaaaattaagaacacaaatataaacattaagttaaaacacaactgaacaaagtgccaaacttaattggagcatctgaaaatgaaacattaggcagttgcagtggcctcttgataataattcacagtaactctctgtaagctgatcctgtctgaagagcagcaggcacaaggcccctggccatgaagtccatctcaaagggccaggctcagcaaagcaggatgcaaacccaggctttccaaataccaggttggggctcatgtcactgtgccacaggagcttctgtagaaaggctacttgaaaaaagtggccattaaaaatccaggtggatcctatctagggcagtgttggaaacactgatctatgggaggaggagcaggaaggaattgtttaaccactgagcagaaatgttacattgctacctgcctttagcagctgtggctgatgggtaccagttgctaagaagagcattacctaacagtgtattaagatagaaaaatgattttaaagcacggcacttagagaatgttgaagttttactttgctttattttgatttgtttggtttgactttgtctcctggagcatcctccatggatttctgttcattacaagagaaacctagggctctaacccaattcctaattcttggacacattgcacccttgttttgtgataatccagccttcttccttgagaaggtttgctggactggaggttacatgtattgaattttctaaaatgaaggtgcaaagctgtctcctcttatttctttgtggtgctcacttcactgtgagatttcctatcaatacagcccaagtcagtgggcatgcatgaggtggagatgagggagttaggaaggacttggactctcatcaaccatcaggatccctgaatccactaactgttcataatcaaagaagtttgaacaaatacttcacacacatgaaattgccaaaattttgcatttgagttgttataccagtaagtccagttgccatcatctccttgtcacaagtgtcttaaattttgcttttgataataatgattaccactcattcagtactaacttacttgatattagacactgcattaaataccttgcaaacattattttgtttgatcctgacaaccatatgagataggtactattcttatccattaccaaaaaaattaatttcatgaagacttttcccagagagagaaactttaaatatttacacacacacctctctccctgtaacaattccgtagtcctgataacagcaaataagcaaagtctgtgtaggatgctttaccaacagtcccacctagaggcaggagagtgaaccagctagaaaatattttattcatatttcttccagaaaggctccattggagtttgaactcaatttatgttataattttcttattatttttgtattggttttcctgaaaccaatacaaagtaagaaagcattggttccactaaaaatgtcctaaaaccagccaagcacagtggctcacacctataatcccagtactttgggaggccgaggcgggtggatcacttaagccaggagttcaagactagcctggccaacatgacgaaaccccatctctactaaaaatacaaaaattagcagggtgtggtagcacacacctgtaatctcagctactcaggaagctgagacatgagaatcgcttgaacctcagaggcagagattacagtgagcagagatcacgccactgtacttctgcctgggtgacagagcgagactctatctaaaaaaaaataaacacataaatagtaaaatgtcctgaaaccattatggggttaaagcaagaggcagggctggttcccaggattttctgtctaatctccagtgagccacagacctattcctgatcaacttgagaataaacacatcagtaaagatgtgtaaggctgtctgactttcccatttctgtagaattttatttgaagagaagtttctcctttctccaggccccatattgtttatacaaaaagacctttccagtaaatgtccacaaccactaccatcaactaaaatgttttcccactaatgctttcaatggtaatcagtatttaacagggcacttaggattattttttgatcaaccattgtttagatattcccacttataattactcctgtgaaggattgcctcggggcatcagctgatcctgagaaattatccagaagccatgagtgtgtaataatttagtcttaaacctaaataggtcagtattgggtgggacttttctcagctgcataatggggagaataaaaagaatatggaaagaagttacgtaacacatcctgggtcacaaacagaggtaagacttgaacacaggcctgacatcaaagcccatgccagtatgacttacaaaaggtagactggactacctgcatttgagtcactagtgatgcttatcactgggcctcaccaaagaaccttggaatcagaatctttggaggtagatgccaggcacctgcattgttatcaagtgctccagtgattaccattcactgtacagagccaaacagactcctgatgctggaagaaaattacagtgctcaaagtgcagggcagggtgtacatctggatctaaatcactgagcaaccacagggtttcaagagagggtcaaaacaaggactttctgctctctgtggccaaggggacactaagtttgcactgttctcagatctccaaagagactttggtgtatgggggatagggaggggggaagggggtgtgaaataaaaggagaaagtgaatttgattatttgattgatgaaaattgaaaagcttattgtagggcctagcctacagttgatgaaaaaacaatggatcaggaagaagatcagaacttgtctcagtcctcaactgttttcctcaggctttggttgaatattgccatcctgtaattcattatagcattttctgttgcataaacgcttagcaacaaagcctttttttaaaaaaatttgtaactcctcaatgaggattaaatgcttcttcttctaagacagtccgaaatatactcacagctgaaaattcagctaaccgcatttcccaactagccacattctatagaaaactctaagccatgcagatgagtacagacttgacaatagtgctcaaggctgggagtactattcatctgaaaagaatgctccctccaattggtgggccgttattctgctaggtttgtgtttggataattataagatggctatgtttttcttccccagtctcaggaggcccaggtgctgaaacaattggcagagaagagggaacacgagcgagaagtccttcagaaggctttggaggagaacaacaacttcagcaagatggcggaggaaaagctgatcctgaaaatggaacaaattaaggaaaaccgtgaggctaatctagctgctattattgaacgtctgcaggaaaaggtaatctcagcagagtcctgagcagatggatatattcatatgcagcacagctgggtgaacttccatatgcctgagcacagagacgaagtcaaaatttgctgcaggtgtgaggacaactaactcccatgggcagggtctcacagtgtagcattgagttagcaggaggtgcaacatggtagagaaatgggaatccatcatgaaagctggaattttgtcaaattttcccatggtgagtggattcagggaggctgattcatgcttttgaaatgtgtaagacttctatacaagcctcacgaggcaatctgtaggaaaaatgttacactggaaatattaatgtctatatattatattgatataagtataaataacatttgatttaatatttgtttaatatatgacattaaatatatatttaattaaaatattaaattagaaaaatatatttgccagaaaaggccagggtatttatgaacactggtaagcccattctagggtataatagcatcacatgggaccatagcaaagattagctcataggggatgtttcatccagttctggtatcctggtgcccttctcttcaacaacctaaacatatattcattcccatgagtcaggaggagctgtgctggagttcttctgaaaaatgctgtctttcacttttgtactctctatgctgtctcccacctatcccctcaaaaaacctttcctttgaaaatatacagtatagctgtgagtagtttagctgtgtccgtttccagaaattggaataagcattgagaaatgggatgtttgagaaagacgcctcaatccttttctgagcagtcagtcacccttcccgccagtagcaagtgcctttgtgtgataggcattggagatgcagagcaaaacaggagtgtgcctgtcatcagagccctgagagtttaattagatgagcctcctgttttctatttctcagagtttcatgtcttctgttagagatggcccttctcatctaaggttcaaaaaaccttatcctgaagttctgatgattctgttttcattctcagtctctgactgcaaatatccaactagaaacaaaggaaatcaggcatgaaaacttttaaagatataattgcatggagatcttcatttgtgctcgtgaggaatttttgaaagcattgctggggaagggtgtgtgggctctgatgcagcagtaagacactgaggctctcagaggtccgtggacgagtactgctgacttgggcaagaaccggaatagttacctgatgccttatccgaaacatgaaagttcggattaaatttgtatttataagctagtgtttttatactctcagaacaatgtcattgcgtttcacccaagtgagtcaagtcacgatttggaagaggcaacagaatttggctctctccaggtgatttatggcggtataggaacacatgttttactcagatacaggggagcaaagttccatttgctaaagtttactcccctgaccttcaaccagtcagtcttcctccatctgccaccactttgcacttctccagagaactaaggatgttcccgcttgaccagtgctcataacatggacagcagagggccactgtgtgatctctttgagatcactgtgactcaaccttcttctcacatcctaggccctaaaacaattaagtgaagttgctaggaacggtacctgctgatcttattgcagcattctcaattaggcctcaatgcaagatttatatcactggcagtcctggagcatttttgtttttcaaattacacatacccaaacacacggcatagcctccttttttgtttgtttgtttttttgagatagagtctcgctgtgtcgcccaggctggagtgcagtggcacgatctcagctcactgcaacctctgcctcctgggttcaagtgattctcatgtctcagcctcccaagtagctgagattacaggcgtataccaccacgcccagctaatttttgtatttttagtagagacagggttttgccgtgttggccaagctggtctcaaactcctgacctcaagtgatccacccacctcggcctcccgaagtgctgggattacaggtgtgagccaccgtgcccagccagggcatatccttcttgatttcaattgtaaaatagttcaaaaattttccatattttatctaatatttccagaagtgctagcttttaacggaccatttttttcctctgtgtgtttttttctcttcacctagcccagccatgctcagctcatttttgtactctttccactcccaaccaaatttagtgccctcccccatacatgcatacatgtacatctgcacaccacttttcctgcaaataatcaacccaaagagtgcttaaaattcctgacatcaacccacagaatctccaaggatgggacccagcatccatacattttaaaaactctccatatagttccaatatgcagccagatttgagaactagtggttcgtagcctgttctgatttaaatctcagctctcagcagtctatcccacgtcacataatgcagcccagagaaattctaggaccacatttttttctggtatttcatagctaatgaggtgcttttcaaatctaataggatctttggccagtgtcagtcaagatcttttatctcctcaataaaaaggaaataccatatttactttgatttgatgtatatcacataggtggatttaatacaaaattgtggtttacatattgtgaatgtgtatactaaaactactttgctttttcctaaaataagacaaagttttatattggaagtaatatttagcattttgtttgaatgaagttactcctattaaattagaaatttaaaagagggtcagtaataacagtaaagccaaaaggcatgacactgccaacgtaacataagctgctctgaaatctaccatatcaaaagataattatgctgggcatggtggctcacacctgtaatcccagcactttgggaggccaaggcaagagaattgcttgaagccaggagttcgagaccagcctgggaaatataatgataccttgcctcaaacaaaaattcaaaaattagccagcagtggtggcacacttgtaaaaatgcctgtagtcatagctacttcagaggctgagatgaaaggattgcttgggccaaggagttcgagactgcactccaacctgggaaatattgtgccactgcactccaacctgggaaacagaacaagaccctgtctctaaaataaaaagaaaaaaaaagatgaccacttctgaaatgacacctatcaatgagttaatcattcaatgaatatgtattgagtccctactatatgcttaggaacctttgtaatatcattaccaaccatgtctttcccaatacagacaatacaaaattcagcaataaataatatagcaccaacaattagagaataagacaacatgtagtatggtccaatatagacagtaaatacaaagacactgaataatatcagtaaaagtaaattcacatcaaggtcactacaccatgcgcccacccttatgatagccctcactggccctatcaattaagcaagagacatgatacaactctgtgcaagcttttccacaatctgcctaccattcagcactcagtcgctcttcccttcaattaagagaattgagcattcaagcatattttcaccatgatgcccataatggtatcttcaatgtcactgactgataaattcccagaaacccctcagagccccagccatgttagctcaaagcctttagctaaaactgaaagcctaaagcaaaagcagccctggctgcacttcggaatctactggacagctctttaagggattctgatttaatgtctggaatagggccaagaaccttgtattattttaaaggctcactagtaggctctaatatttagccgtggttgagaaccactgtgctaaatgtttcttaaatatgctttgtgatgtcatcataaattatattttagtattttttgtctttgttgcataagtgttctttcttcctccaaagaagaatgttacactcatttcttatttcagtttcctgttttcatagcacctcatcttaacactccaggctattatatagaaaagaatcaaatgtggagaaggctgtgggagaagggatgcctgtgccacaaaggcctgcattaggctgacctattgatgtcatatccaggactcaaaagactagtctgtggattatgactggtgaagttcaaaatgttcttattcttagagtggtatgagaagtagaaagagagagaaacagagaaggggaggagaggggaagagaggaagatgagagaaaggaaagagagggggaaacacctgttcttgacatacaggaatgattcaagacattttcttcctcccctgatgtgtccctttctcccctaacgcactatgcagcatcctgcagaaaattcaccacctgacccttttagaaaccctgagtagtaggagcgccaaatgacccaatcaagaattgcagtgagacagttagttttgaaaaatcagttaaagcatgtataatcattttaacaacaatacatctattcactaaacatataattttaatgtcaaatatttacgtgtaaacatattgaccaatctttcgatgtagttgggcccaataccttttccaaaaattgatcagttaatgggggttctatgggggtttcttttcttgccattattcacacttatgtcacattagctatgatttgcagttttaatttctttaaaattgagtagggactaaagacatctccaaaaagcctggatatagactttttacaacttttccatagcttttatagttgactcacccagtatctactaaatacttcactttctcacgtatttccaaaggtttctctccaccctcacaattttccattaatgtagtacttaattaaattagatagttaaattttcaaatgtgaattgctaaacaggtgtggaaataccattggctataatcaagcatataacacaaccatttgagaaggaaagtatgtggcaatattagggaagagccctttcctctcaagcaattcagcatttaggaaccatcagacagcaggacgatggagggaacagagagggttaacatggcaagttactgaagaggacttctactgaatcttgttgaattccccacttaatccagattgtatcatatcttctttcttttgtaattctaccatatcatcttagtcaatgccaagacttctgagctcataacatggtaacaaataccaaaggagctttcagtatcgtttagaaaggagagaagcaagtaacccagacaaacttgacaactgctttcccctatccaaccatgaagtacagtacttaggaaataaaagaaattgcttcactataattcatcatttcacttctaatatctagaaaatgtcaaatgaaaatattatagccatattttagtggcaatagtagcacataatatgatgcaacttaaaatgataaaaatattttcagggaataagattctgtgattctttccctaagaggtaattttgataatatgtacctgttttgtaaatgtcaatagtcttggggatacaggtggtgtttggttacatggaaaagttccttagtggtgatttctgagattttagtgcacccaatacccaagcagtgtacactgtacccaatatgtagtctttcatccctcgcccccactcccaaccttcccccacaagtctctaaagtccattatatcactcttatatctttgcatactcatagcttagctcccacttatgagaacatatgatagttagtgctcaattcctgagttacttcacttagaataatggcctccagctccacccaagttgctgcaaaagacactatttagttcctttttatggctgagtagtattacatggtgtatatataccacattttatttatccacttgttggtcaatggacacttaacattagttccatatctttgtaatttcaagttgtgctgctataagcatgcatgagcctgtgtctttttcatataattacttcttttcctttgggtagatacccagcagtgggattgctggatcaaatgatagttctactttcagttctttatgttttccacagtggtcatactaatttacattcccatcaacagtgtaaagtgttcccttttcatcacacccatgccaacacctattgttttttgactttttaattacggccattcttgcaggagtaaggtggtatttcattgtggttttaatttgcatttccctgatgttgacaatatttaactctttagttatagattccagctattatcaatttacacctattgcattcttctcatcttttgttttcttgtgattctgatgcacaaatatcatttgtgcaaccacttactgttgaacatgtctgatgaacacttactattgaacatgtctgatgaatgaataatgaaataggaaaagggattaaaactagcctttattaattgtttgctataggccagacatttttggatgtactatcacatttcatccaaacaacaacctaaaagaaaatactgtgattatccccatttcacatctaaggaacctggtctttaggaagattaagtcatttggccaagatcacaagtagaccacagagactagatttgaatgcaagtctgtttgactccaaacctttttactatctgcccatgacccctgatcaccaacatctcaatgtatgaacatgtgctttcttagctcacacaactcactcctgaccccttttttatattgcaagtgcatagtcattagtaaaaagaaggatttttgatgatactgacctcatcttgaatttaattaggctcatatgacagaattccatagatggaattgacatcctaggtcatatagtccaagtccttgtttatatttgatacctagtgagattaaagggacattaaaaagtaaagaaaggaaagacctcatatttcttaccttccagtagagaaatctttctatgaaatcagaggaaagaattagaggaccagaatttttcctaaaatcaactttcatacatcttttttcatataaaaggcatagctgcatacaatgctaaaatattgtattacatttcctttatattgatgggaggaagggggtaaattgcagaaaacattgtaaatttagatatgcttgggcctctgacagtgcctagcaaatatcaggagatcaataatgaaataaatattatcaaagagtagtcttcttgatgaaccttctctgagtatcacaactgctttaggaacctctagattcaaggtctagtaattgcaaacagtgagctgataagaaaaacagactgtatgggaaattacatgcttcctgcatgactgccttttgttctcccacattttgatataaagtcacattaacagttcatgagtaaatattcgataatgtgaacgtaaagtgttcaaataatagagtgactaaaatgcctgaaaacaaataatttttaattagaaactcataatcatttattttctctttttccacattatctcaagctcacaaattatatttattctttcctatggcaaaatccattttgttaacactaattttgagtttaacaagaagtgtactccaaagtagcctaataatactaattataatgtttcctgctatgttatcagtttgaatttatatgaatctttagacttgaggcttctttttcctagcatagtgatggtctgggctttttctcaatttttgccagagctcagctctcactaattagtttctttctgcatgagaaaaagattttgcttcatctttttccttataatagcagaacaaaaagaagaatcagctgcatccatgctaatttcccctgtgacatttccaaacaggatttgatttctctatgcatgcctctttccttctcttcatggtttttgaacatatacaaaagctcatttaaaccaattaaataaaattgtttttaatctctttctctagagtcaacttcctgcttactccaactctgtatctttgaaggaagtatagggtggtctatgccttttttctcccagaatctacacttgaaaagacacatttttccatgcaactataaaatgttctcctcactcaacattgaaattgtatagcagtgattaagagagtgagctgtagagccaggttccctgggtttaaatcccacttgttagtatcatgaagatgggcaagttacttacccttcctgtgtttcagtttcttcatctgcaaaatggggacaataatagaatgtccactataagattattgtgaggattaagggaattaatacaggtaaaacgtgtactgatgcaggtctggtacacattaagtgcctaataaatattcagtattatgatataaagaaccctataagtgtagactccttgagattaatagagtttaacgataagttttactttatagctggtcaagtttatttcttctgaactaaaagaatctatagagtctcaatttctggagcttcagagggaaggagagaagcaatgtaagcaacattctacagaaatataaataatactactaataattagcatcttaaaatttcaattcaatgaacatttatttagcgcctatgatatatgcaagacagtttgattttagtcatctgatgtatagccacatactaaaaaatactgattttagtcatctgatgtatagccacatactaaaaaatacttcctccatcagttccctcctcaggaagttcagttcccaatcccaggctagtaccttggttccttatgtaaataaacatccaccaattacatgctatctgcaaagcactctgctaggccctgcaaatggaaaaaaaaatgataaaacatagtccaggccctcaatgagcttacagtcaaatataatagaggagacaagaacagagaggctcataatacaactagaataaaatgactgccgaataaaaggaaagatttatgcaggtgttcaaatggaaagtgagataagtttgcaggttagtctttgcagtctcataaaaatctttatggagaaaaggacaatggtcatagggcttaaagagtaagtttataatcctgaccagtggagatgaaagactagcattgaaaattgcatgacaagacaattccattaaactgaaacatcaagtgtgtgtaggaaaagatgggggttatgactggaaacgtcacttggactgcaattatgaagggccttgacaaacaggtcaagagtttaagaagcagtatagaaagtcttcgtcctggatctagccctcccagagtgtccatcaggattataaagtccttaaaatattagtcaaaaggaacgacatcattagaaatgatagagaaacaataatgtgatgttttattacctttctctggatttatactctgatcctaatattcaaaactatcttaataacatgaacttttggtcatagttttaaacaaaaacagtgttaaatatattttttaaaacacagtaagtcttgtaagatcttttctaacatgacattttgcagggcccatattttccttctgaaatgggaaaaattcataaaagtagacaccaaactgggttacttctagtcaagcgcatggtacgcaaaggaccagacaaaaagggcctgtgacatttcttcttccttttgtgttttttaggagaggcatgctgcggaggtgcgcaggaacaaggaactccaggttgaactgtctggctgaagcaagggagggtctggcacgccccaccaatagtaaatccccctgcctatattataatggatcatgcgatatcaggatggggaatgtatgacatggtttaaaaagaactcattataaaaaaaaaaaaacaaaaaaaatcaaaaattaaaaaaaatcaatgcggtctctttgcagaatgttttgcttgatgtttaaaaaataccttggatcttattttgtaaatacttacatttttgttaaaaaatacaagtattgcattatgcaagttatttcataatcttacatgtcctgtaacaggcttttgatgttgtgtctttccactcaaatgaatttgctaggtctgttctttttgaagctccccatgtctaactccattccaaaagaaaaatgaggtcagtagacagtctatggtgctagaaacccaccattgcctaatgacctagaaggctttgttgtctctgagcttgactaagaccatacctagatcacaggtattatgactccacatgaaccttcacatttgttcgctcataatctacttactgcctaaaaactacaaaaccaggctaagaaataccaccagtcatagcatttacttctgcttctcctggattatgtgctacaaatgtgctttggctttagaaagggatggatgagaagacagacctgagaccaatctgggtagaagcaaaaagttgaaccttttaaagtgctgaacacaaatccaaattcgaatggttcaagcagccgtgaaatcgctcttcataaagtgggcttaattctctagtttaagttcttttgatggaatgaattaattaatgtgtcaggtggcttatttgtggatgccatgattgatgatgttcattttaagctcttacctatagtacaagtacatgatgctactgaatatttttccacttggaaactgtgagctggttgttgcattaaaacacacatacaaacaaaatcaaaaacactgcggactttcactcaagctggtctttcttccccagtgtaaggcaatcctgcctactaacaacaccaacaacaaaacactccatctgtgaagctgacgcagttaagggggctaggcagggcatttgtgccaactaagaatcaccagatacccaccataagtacctatcgcagttttgaagtcgtttctccccaactcccaactcctgaaggttgctgcctgcatatttactcttcattagtgctattttcctgtatgtcattgtgagcaagctgtgattaataaagaattggagttctgtgaactaataaaggtttggtctgttctcttgcctcttcatgtgtctgaccctggagttcagattttaaaagggcagccactgaattagacaatctcacaagcaaagagataataaggagagtttgaaggaaaccagtgccctgctgagtcatttccctttgggaacagcaggccgcatctcttttctcttgccctctgatgggccaacactgtgggagaggtcttcaatctgctccctttaaaaacatacaggactgaacaagataccacaaaatgcagactgataattctgaggaagtgagcccttttcaaaacattgaaggttcctggcattatctcaagttctcccctgtggcccacagaacgcctcccccagcctaggccgtctgttaagatgtgatccctgacgtactgctgaattttgtactgccaataaacacactgcacacatgggcgcagcagggtcctgtcagtgacccaaatggatcttacaggaccaaaggaaccacatccagttttagcagaaaatcataaataattgaaaccacacagatttgtgaataagtacagtgggctatggtccataggagctctaacaagccaagtttagggttgcggccacctggtctctattttgaatatcctttaatatttgtgggaatctaatactgctacaaacaatgcctgagcttgatgtgaaacacatgagcttccaatgagcatgaaatgagttgtctcttcatactggttttctatagcttagtttggaaaacattacccgaattactcattgggtcagaccctatgataagccctggaaacatgaacatgaatgaccacaagaagctcataatccagtagcttatttgggccccaaaacagcaaacatgatatgttggtcagccctctgcagcatatttggggaaaatggatcatttctaagaagtttctgaaatattagaaatatttgacaaacttctcatagtactttgtatgagaactgaaactgctgaccagtccagctgcctaatctcaaagatgaagaagcaggccgggcacggtggctcatgcctgtaatcccaacactttgggaggccagggcaggcagatcacttgaagggaggagttcgagaccagcctgggaaacaaggtgaaatatcttttctacaaaaaaaaatacaaaaagtagccaggcatgctggggagtaactgtagtctcagctacttgaaaggatgagataggagaatgacctgagcctggggagatcaaggctgcagtgagctgagatcacaccgctgcactccagcctgggtgacagaacgagaccatgtctcaaaaaaaaaaaaaaaaaaaaaaagcgaaaaagcagaggagggaaaggtgagtggttggctccagaccacagtgcccacatctcatgacttcttcttcccttatttgaggtcctcagaagaacatctccaagttgggtgcagcatatactgcttgggtgatgggtgtaccaaactctcacaaataaccacgaaagtacttactcatgtaaccaaataccacctgtttcccaaaaacctatagaaataaaaaagcaaaaaaaaatttaaaagaaaaataaataaataaaagagcataattcacaatggggggaaaaaaagaacacctccaaacctgccagaccagtgaggccagagggcctcaaatgtcagtgtgcttccaagttatgtgaacggcttgttaaaaagagatttctgagtcctgcctccagagctttggattcctgaggtctctggaagcctgaaaatgtgcatttctaacatattcccagaagatgctaatactgcttgtctgcagccctcacttgagaagccctgctgtcaacaatgcaactgatccaggtgtgtggattctctgtggctgccacactgagggaatctgtttcaatcagggcatgttccattggaggcagggcaagaagagaaatgcattcactcttattcaaaaaagttcgggggtgggaggatgcagagaaatccagaaaaaagcaagataaacagctgtgccttgcaggataataaaacaatggaagcaatcaatcgtgcagccaggggctgggatagagctaaggtggaatcagagtaccaatgactcggcatctctcacacaacatgatttcagtaaggatcaaaaatctgaagacgctccccaccctttaatagatgaactgagtgctttttgcaaggatttgccattctgtgaattacccagtttgttagcctggttccagttacagattttttgtttgtttgtttgtttgttttacggagtcttggcttcttcagtactgtgtttaaattgcttaaatgtctttgaaatgttctgtgaaaattcacaaagttccattagtttgatgattcaccctcctcccctatctctcctacacccaaaaatgtatggaacaactgaatgttgacaaaggtcattctcttttctagcaagagtctcgcctttgtgctcttcatcagccttcacaacacctgagaggagaaaacaatgggggtaaaagtgctaataggcgtattgtggcccatctaccctcctcctactgagggagaggatgaaaagggggagacgtgacttctgtggctttagcagccaaataacacatcatctctctcctcccagcccctggacaagacatccttgacgtctaatcaggcagtttgctgaaactgctaaggcaagcacagagcccagaacaaagctaaggcccccaagctcccccacattacctgagtcgtcttggattcctcactgtcccccaccacccacctccagttgaatatcaaatggtg

SEQ ID NO: 2 - RNA, 341 nt, Homo sapiens
  source 1..341: mol_type = other RNA
ttccgaactcatatacctggggattttattactctgggaattatgtgttctgccccatcactctctcttaattggatttttaaaattatattcatattgcaggactcggcagaagaccttcgagagaaaggtagaaaataagaatttggctctctgtgtgagcatgtgtgcgtgtgtgcgagagagagagacagacagcctgcctaagaagaaatgaatgtgaatgcggcttgtggcacagttgacaaggatgataaatcaataatgcaagcttactatcatttatgaatagcaatactgaagaaattaaaacaaaagattgctgtctcaatatatcttat

SEQ ID NO: 3 - DNA, 21 bp, Synthetic construct
  source 1..21: mol_type = other DNA
ccacgaactttagcttctcca

SEQ ID NO: 4 - DNA, 20 bp, Synthetic construct
  source 1..20: mol_type = other DNA
gccaattgtttcagcacctg

SEQ ID NO: 5 - DNA, 26 bp, Synthetic construct
  source 1..26: mol_type = other DNA
actttcttctttcctctgcagcctcc

SEQ ID NO: 6 - DNA, 21 bp, Synthetic construct
  source 1..21: mol_type = other DNA
aaggcaatcctgcctactaac

SEQ ID NO: 7 - DNA, 23 bp, Synthetic construct
  source 1..23: mol_type = other DNA
ggtgggtatctggtgattcttag

SEQ ID NO: 8 - DNA, 24 bp, Synthetic construct
  source 1..24: mol_type = other DNA
tccatctgtgaagctgacgcagtt

SEQ ID NO: 9 - skipped

SEQ ID NO: 10 - DNA, 18 bp, Synthetic construct
  source 1..18: mol_type = other DNA
aataaaatccccaggtat

SEQ ID NO: 11 - DNA, 18 bp, Synthetic construct
  source 1..18: mol_type = other DNA
gagtaataaaatccccag

SEQ ID NO: 12 - DNA, 18 bp, Synthetic construct
  source 1..18: mol_type = other DNA
cccagagtaataaaatcc

SEQ ID NO: 13 - DNA, 18 bp, Synthetic construct
  source 1..18: mol_type = other DNA
aattcccagagtaataaa

SEQ ID NO: 14 - DNA, 18 bp, Synthetic construct
  source 1..18: mol_type = other DNA
acataattcccagagtaa

SEQ ID NO: 15 - DNA, 18 bp, Synthetic construct
  source 1..18: mol_type = other DNA
gaacacataattcccaga

SEQ ID NO: 16 - DNA, 18 bp, Synthetic construct
  source 1..18: mol_type = other DNA
ggcagaacacataattcc

SEQ ID NO: 17 - DNA, 18 bp, Synthetic construct
  source 1..18: mol_type = other DNA
atggggcagaacacataa

SEQ ID NO: 18 - DNA, 18 bp, Synthetic construct
  source 1..18: mol_type = other DNA
agtgatggggcagaacac

SEQ ID NO: 19 - DNA, 18 bp, Synthetic construct
  source 1..18: mol_type = other DNA
agagagtgatggggcaga

SEQ ID NO: 20 - DNA, 18 bp, Synthetic construct
  source 1..18: mol_type = other DNA
taagagagagtgatgggg

SEQ ID NO: 21 - DNA, 18 bp, Synthetic construct
  source 1..18: mol_type = other DNA
caattaagagagagtgat

SEQ ID NO: 22 - DNA, 18 bp, Synthetic construct
  source 1..18: mol_type = other DNA
aatccaattaagagagag

SEQ ID NO: 23 - DNA, 18 bp, Synthetic construct
  source 1..18: mol_type = other DNA
taaaaatccaattaagag

SEQ ID NO: 24 - DNA, 18 bp, Synthetic construct
  source 1..18: mol_type = other DNA
attttaaaaatccaatta

SEQ ID NO: 25 - DNA, 18 bp, Synthetic construct
  source 1..18: mol_type = other DNA
tataattttaaaaatcca

SEQ ID NO: 26 - DNA, 18 bp, Synthetic construct
  source 1..18: mol_type = other DNA
tgaatataattttaaaaa

SEQ ID NO: 27 - DNA, 18 bp, Synthetic construct
  source 1..18: mol_type = other DNA
aatatgaatataatttta

SEQ ID NO: 28 - DNA, 18 bp, Synthetic construct
  source 1..18: mol_type = other DNA
ctgcaatatgaatataat

SEQ ID NO: 29 - DNA, 18 bp, Synthetic construct
  source 1..18: mol_type = other DNA
agtcctgcaatatgaata

SEQ ID NO: 30 - DNA, 18 bp, Synthetic construct
  source 1..18: mol_type = other DNA
gccgagtcctgcaatatg

SEQ ID NO: 31 - DNA, 18 bp, Synthetic construct
  source 1..18: mol_type = other DNA
ttctgccgagtcctgcaa

SEQ ID NO: 32 - DNA, 18 bp, Synthetic construct
  source 1..18: mol_type = other DNA
ggtcttctgccgagtcct

SEQ ID NO: 33 - DNA, 18 bp, Synthetic construct
  source 1..18: mol_type = other DNA
cgaaggtcttctgccgag

SEQ ID NO: 34 - DNA, 18 bp, Synthetic construct
  source 1..18: mol_type = other DNA
ctctcgaaggtcttctgc

SEQ ID NO: 35 - DNA, 18 bp, Synthetic construct
  source 1..18: mol_type = other DNA
ctttctctcgaaggtctt

SEQ ID NO: 36 - DNA, 18 bp, Synthetic construct
  source 1..18: mol_type = other DNA
ctacctttctctcgaagg

SEQ ID NO: 37 - DNA, 18 bp, Synthetic construct
  source 1..18: mol_type = other DNA
ttttctacctttctctcg

SEQ ID NO: 38 - DNA, 18 bp, Synthetic construct
  source 1..18: mol_type = other DNA
cttattttctacctttct

SEQ ID NO: 39 - DNA, 18 bp, Synthetic construct
  source 1..18: mol_type = other DNA
aattcttattttctacct

SEQ ID NO: 40 - DNA, 18 bp, Synthetic construct
  source 1..18: mol_type = other DNA
gccaaattcttattttct

SEQ ID NO: 41 - DNA, 18 bp, Synthetic construct
  source 1..18: mol_type = other DNA
gagagccaaattcttatt

SEQ ID NO: 42 - DNA, 18 bp, Synthetic construct
  source 1..18: mol_type = other DNA
cacagagagccaaattct

SEQ ID NO: 43 - DNA, 18 bp, Synthetic construct
  source 1..18: mol_type = other DNA
ctcacacagagagccaaa

SEQ ID NO: 44 - DNA, 18 bp, Synthetic construct
  source 1..18: mol_type = other DNA
catgctcacacagagagc

SEQ ID NO: 45 - DNA, 18 bp, Synthetic construct
  source 1..18: mol_type = other DNA
cacacatgctcacacaga

SEQ ID NO: 46 - DNA, 18 bp, Synthetic construct
  source 1..18: mol_type = other DNA
cacgcacacatgctcaca

SEQ ID NO: 47 - DNA, 18 bp, Synthetic construct
  source 1..18: mol_type = other DNA
cacacacgcacacatgct

SEQ ID NO: 48 - DNA, 18 bp, Synthetic construct
  source 1..18: mol_type = other DNA
ctcgcacacacgcacaca

SEQ ID NO: 49 - DNA, 18 bp, Synthetic construct
  source 1..18: mol_type = other DNA
ctctctcgcacacacgca

SEQ ID NO: 50 - DNA, 18 bp, Synthetic construct
  source 1..18: mol_type = other DNA
ctctctctctcgcacaca

SEQ ID NO: 51 - DNA, 18 bp, Synthetic construct
  source 1..18: mol_type = other DNA
ctgtctctctctctcgca

SEQ ID NO: 52 - DNA, 18 bp, Synthetic construct
  source 1..18: mol_type = other DNA
ctgtctgtctctctctct

SEQ ID NO: 53 - DNA, 18 bp, Synthetic construct
  source 1..18: mol_type = other DNA
caggctgtctgtctctct

SEQ ID NO: 54 - DNA, 18 bp, Synthetic construct
  source 1..18: mol_type = other DNA
taggcaggctgtctgtct

SEQ ID NO: 55 - DNA, 18 bp, Synthetic construct
  source 1..18: mol_type = other DNA
ttcttaggcaggctgtct

SEQ ID NO: 56 - DNA, 18 bp, Synthetic construct
  source 1..18: mol_type = other DNA
tttcttcttaggcaggct

SEQ ID NO: 57 - DNA, 18 bp, Synthetic construct
  source 1..18: mol_type = other DNA
ttcatttcttcttaggca

SEQ ID NO: 58 - DNA, 18 bp, Synthetic construct
  source 1..18: mol_type = other DNA
cacattcatttcttctta

SEQ ID NO: 59 - DNA, 18 bp, Synthetic construct
  source 1..18: mol_type = other DNA
cattcacattcatttctt

SEQ ID NO: 60 - DNA, 18 bp, Synthetic construct
  source 1..18: mol_type = other DNA
gccgcattcacattcatt

SEQ ID NO: 61 - DNA, 18 bp, Synthetic construct
  source 1..18: mol_type = other DNA
acaagccgcattcacatt

SEQ ID NO: 62 - DNA, 18 bp, Synthetic construct
  source 1..18: mol_type = other DNA
tgccacaagccgcattca

SEQ ID NO: 63 - DNA, 18 bp, Synthetic construct
  source 1..18: mol_type = other DNA
actgtgccacaagccgca

SEQ ID NO: 64 - DNA, 18 bp, Synthetic construct
  source 1..18: mol_type = other DNA
gtcaactgtgccacaagc

SEQ ID NO: 65 - DNA, 18 bp, Synthetic construct
  source 1..18: mol_type = other DNA
ccttgtcaactgtgccac

SEQ ID NO: 66 - DNA, 18 bp, Synthetic construct
  source 1..18: mol_type = other DNA
tcatccttgtcaactgtg

SEQ ID NO: 67 - DNA, 18 bp, Synthetic construct
  source 1..18: mol_type = other DNA
tttatcatccttgtcaac

SEQ ID NO: 68 - DNA, 18 bp, Synthetic construct
  source 1..18: mol_type = other DNA
ttgatttatcatccttgt

SEQ ID NO: 69 - DNA, 18 bp, Synthetic construct
  source 1..18: mol_type = other DNA
attattgatttatcatcc

SEQ ID NO: 70 - DNA, 18 bp, Synthetic construct
  source 1..18: mol_type = other DNA
ttgcattattgatttatc

SEQ ID NO: 71 - DNA, 18 bp, Synthetic construct
  source 1..18: mol_type = other DNA
aagcttgcattattgatt

SEQ ID NO: 72 - DNA, 18 bp, Synthetic construct
  source 1..18: mol_type = other DNA
tagtaagcttgcattatt

SEQ ID NO: 73 - DNA, 18 bp, Synthetic construct
  source 1..18: mol_type = other DNA
atgatagtaagcttgcat

SEQ ID NO: 74 - DNA, 18 bp, Synthetic construct
  source 1..18: mol_type = other DNA
ataaatgatagtaagctt

SEQ ID NO: 75 - DNA, 18 bp, Synthetic construct
  source 1..18: mol_type = other DNA
attcataaatgatagtaa

SEQ ID NO: 76 - DNA, 18 bp, Synthetic construct
  source 1..18: mol_type = other DNA
tgctattcataaatgata

SEQ ID NO: 77 - DNA, 18 bp, Synthetic construct
  source 1..18: mol_type = other DNA
gtattgctattcataaat

SEQ ID NO: 78 - DNA, 18 bp, Synthetic construct
  source 1..18: mol_type = other DNA
ttcagtattgctattcat

SEQ ID NO: 79 - DNA, 18 bp, Synthetic construct
  source 1..18: mol_type = other DNA
tttcttcagtattgctat

SEQ ID NO: 80 - DNA, 18 bp, Synthetic construct
  source 1..18: mol_type = other DNA
ttaatttcttcagtattg

SEQ ID NO: 81 - DNA, 18 bp, Synthetic construct
  source 1..18: mol_type = other DNA
tgttttaatttcttcagt

SEQ ID NO: 82 - DNA, 18 bp, Synthetic construct
  source 1..18: mol_type = other DNA
cttttgttttaatttctt

SEQ ID NO: 83 - DNA, 18 bp, Synthetic construct
  source 1..18: mol_type = other DNA
caatcttttgttttaatt

SEQ ID NO: 84 - DNA, 18 bp, Synthetic construct
  source 1..18: mol_type = other DNA
acagcaatcttttgtttt

SEQ ID NO: 85 - DNA, 18 bp, Synthetic construct
  source 1..18: mol_type = other DNA
tgagacagcaatcttttg

SEQ ID NO: 86 - DNA, 18 bp, Synthetic construct
  source 1..18: mol_type = other DNA
atattgagacagcaatct

SEQ ID NO: 87 - DNA, 18 bp, Synthetic construct
  source 1..18: mol_type = other DNA
agatatattgagacagca

SEQ ID NO: 88 - DNA, 18 bp, Synthetic construct
  source 1..18: mol_type = other DNA
taataaaatccccaggta

SEQ ID NO: 89 - DNA, 18 bp, Synthetic construct
  source 1..18: mol_type = other DNA
agagtaataaaatcccca

SEQ ID NO: 90 - DNA, 18 bp, Synthetic construct
  source 1..18: mol_type = other DNA
tcccagagtaataaaatc

SEQ ID NO: 91 - DNA, 18 bp, Synthetic construct
  source 1..18: mol_type = other DNA
taattcccagagtaataa

SEQ ID NO: 92 - DNA, 18 bp, Synthetic construct
  source 1..18: mol_type = other DNA
cacataattcccagagta

SEQ ID NO: 93 - DNA, 18 bp, Synthetic construct
  source 1..18: mol_type = other DNA
agaacacataattcccag

SEQ ID NO: 94 - DNA, 18 bp, Synthetic construct
  source 1..18: mol_type = other DNA
gggcagaacacataattc

SEQ ID NO: 95 - DNA, 18 bp, Synthetic construct
  source 1..18: mol_type = other DNA
gatggggcagaacacata

SEQ ID NO: 96 - DNA, 18 bp, Synthetic construct
  source 1..18: mol_type = other DNA
gagtgatggggcagaaca

SEQ ID NO: 97 - DNA, 18 bp, Synthetic construct
  source 1..18: mol_type = other DNA
gagagagtgatggggcag

SEQ ID NO: 98 - DNA, 18 bp, Synthetic construct
  source 1..18: mol_type = other DNA
ttaagagagagtgatggg

SEQ ID NO: 99 - DNA, 18 bp, Synthetic construct
  source 1..18: mol_type = other DNA
ccaattaagagagagtga

SEQ ID NO: 100 - DNA, 18 bp, Synthetic construct
  source 1..18: mol_type = other DNA
aaatccaattaagagaga

SEQ ID NO: 101 - DNA, 18 bp, Synthetic construct
  source 1..18: mol_type = other DNA
ttaaaaatccaattaaga

SEQ ID NO: 102 - DNA, 18 bp, Synthetic construct
  source 1..18: mol_type = other DNA
aattttaaaaatccaatt

SEQ ID NO: 103 - DNA, 18 bp, Synthetic construct
  source 1..18: mol_type = other DNA
atataattttaaaaatcc

SEQ ID NO: 104 - DNA, 18 bp, Synthetic construct
  source 1..18: mol_type = other DNA
atgaatataattttaaaa

SEQ ID NO: 105 - DNA, 18 bp, Synthetic construct
  source 1..18: mol_type = other DNA
caatatgaatataatttt

SEQ ID NO: 106 - DNA, 18 bp, Synthetic construct
  source 1..18: mol_type = other DNA
cctgcaatatgaatataa

SEQ ID NO: 107 - DNA, 18 bp, Synthetic construct
  source 1..18: mol_type = other DNA
gagtcctgcaatatgaat

SEQ ID NO: 108 - DNA, 18 bp, Synthetic construct
  source 1..18: mol_type = other DNA
tgccgagtcctgcaatat

SEQ ID NO: 109 - DNA, 18 bp, Synthetic construct
  source 1..18: mol_type = other DNA
cttctgccgagtcctgca

SEQ ID NO: 110 - DNA, 18 bp, Synthetic construct
  source 1..18: mol_type = other DNA
aggtcttctgccgagtcc

SEQ ID NO: 111 - DNA, 18 bp, Synthetic construct
  source 1..18: mol_type = other DNA
tcgaaggtcttctgccga

SEQ ID NO: 112 - DNA, 18 bp, Synthetic construct
  source 1..18: mol_type = other DNA
tctctcgaaggtcttctg

SEQ ID NO: 113 - DNA, 18 bp, Synthetic construct
  source 1..18: mol_type = other DNA
cctttctctcgaaggtct

SEQ ID NO: 114 - DNA, 18 bp, Synthetic construct
  source 1..18: mol_type = other DNA
tctacctttctctcgaag

SEQ ID NO: 115 - DNA, 18 bp, Synthetic construct
  source 1..18: mol_type = other DNA
attttctacctttctctc

SEQ ID NO: 116 - DNA, 18 bp, Synthetic construct
  source 1..18: mol_type = other DNA
tcttattttctacctttc

SEQ ID NO: 117 - DNA, 18 bp, Synthetic construct
  source 1..18: mol_type = other DNA
aaattcttattttctacc

SEQ ID NO: 118 - DNA, 18 bp, Synthetic construct
  source 1..18: mol_type = other DNA
agccaaattcttattttc

SEQ ID NO: 119 - DNA, 18 bp, Synthetic construct
  source 1..18: mol_type = other DNA
agagagccaaattcttat

SEQ ID NO: 120 - DNA, 18 bp, Synthetic construct
  source 1..18: mol_type = other DNA
acacagagagccaaattc

SEQ ID NO: 121 - DNA, 18 bp, Synthetic construct
  source 1..18: mol_type = other DNA
gctcacacagagagccaa

SEQ ID NO: 122 - DNA, 18 bp, Synthetic construct
  source 1..18: mol_type = other DNA
acatgctcacacagagag

SEQ ID NO: 123 - DNA, 18 bp, Synthetic construct
  source 1..18: mol_type = other DNA
gcacacatgctcacacag

SEQ ID NO: 124 - DNA, 18 bp, Synthetic construct
  source 1..18: mol_type = other DNA
acacgcacacatgctcac

SEQ ID NO: 125 - DNA, 18 bp, Synthetic construct
  source 1..18: mol_type = other DNA
gcacacacgcacacatgc

SEQ ID NO: 126 - DNA, 18 bp, Synthetic construct
  source 1..18: mol_type = other DNA
tctcgcacacacgcacac

SEQ ID NO: 127 - DNA, 18 bp, Synthetic construct
  source 1..18: mol_type = other DNA
tctctctcgcacacacgc

SEQ ID NO: 128 - DNA, 18 bp, Synthetic construct
  source 1..18: mol_type = other DNA
tctctctctctcgcacac

SEQ ID NO: 129 - DNA, 18 bp, Synthetic construct
  source 1..18: mol_type = other DNA
tctgtctctctctctcgc

SEQ ID NO: 130 - DNA, 18 bp, Synthetic construct
  source 1..18: mol_type = other DNA
gctgtctgtctctctctc

SEQ ID NO: 131 - DNA, 18 bp, Synthetic construct
  source 1..18: mol_type = other DNA
gcaggctgtctgtctctc

SEQ ID NO: 132 - DNA, 18 bp, Synthetic construct
  source 1..18: mol_type = other DNA
ttaggcaggctgtctgtc

SEQ ID NO: 133 - DNA, 18 bp, Synthetic construct
  source 1..18: mol_type = other DNA
cttcttaggcaggctgtc

SEQ ID NO: 134 - DNA, 18 bp, Synthetic construct
  source 1..18: mol_type = other DNA
atttcttcttaggcaggc

SEQ ID NO: 135 - DNA, 18 bp, Synthetic construct
  source 1..18: mol_type = other DNA
attcatttcttcttaggc

SEQ ID NO: 136 - DNA, 18 bp, Synthetic construct
  source 1..18: mol_type = other DNA
tcacattcatttcttctt

SEQ ID NO: 137 - DNA, 18 bp, Synthetic construct
  source 1..18: mol_type = other DNA
gcattcacattcatttct

SEQ ID NO: 138 - DNA, 18 bp, Synthetic construct
  source 1..18: mol_type = other DNA
agccgcattcacattcat

SEQ ID NO: 139 - DNA, 18 bp, Synthetic construct
  source 1..18: mol_type = other DNA
cacaagccgcattcacat

SEQ ID NO: 140 - DNA, 18 bp, Synthetic construct
  source 1..18: mol_type = other DNA
gtgccacaagccgcattc

SEQ ID NO: 141 - DNA, 18 bp, Synthetic construct
  source 1..18: mol_type = other DNA
aactgtgccacaagccgc

SEQ ID NO: 142 - DNA, 18 bp, Synthetic construct
  source 1..18: mol_type = other DNA
tgtcaactgtgccacaag

SEQ ID NO: 143 - DNA, 18 bp, Synthetic construct
  source 1..18: mol_type = other DNA
tccttgtcaactgtgcca

SEQ ID NO: 144 - DNA, 18 bp, Synthetic construct
  source 1..18: mol_type = other DNA
atcatccttgtcaactgt

SEQ ID NO: 145 - DNA, 18 bp, Synthetic construct
  source 1..18: mol_type = other DNA
atttatcatccttgtcaa

SEQ ID NO: 146 - DNA, 18 bp, Synthetic construct
  source 1..18: mol_type = other DNA
attgatttatcatccttg

SEQ ID NO: 147 - DNA, 18 bp, Synthetic construct
  source 1..18: mol_type = other DNA
cattattgatttatcatc

SEQ ID NO: 148 - DNA, 18 bp, Synthetic construct
  source 1..18: mol_type = other DNA
cttgcattattgatttat

SEQ ID NO: 149 - DNA, 18 bp, Synthetic construct
  source 1..18: mol_type = other DNA
taagcttgcattattgat

SEQ ID NO: 150 - DNA, 18 bp, Synthetic construct
  source 1..18: mol_type = other DNA
atagtaagcttgcattat

SEQ ID NO: 151 - DNA, 18 bp, Synthetic construct
  source 1..18: mol_type = other DNA
aatgatagtaagcttgca

SEQ ID NO: 152 - DNA, 18 bp, Synthetic construct
  source 1..18: mol_type = other DNA
cataaatgatagtaagct

SEQ ID NO: 153 - DNA, 18 bp, Synthetic construct
  source 1..18: mol_type = other DNA
tattcataaatgatagta

SEQ ID NO: 154 - DNA, 18 bp, Synthetic construct
  source 1..18: mol_type = other DNA
ttgctattcataaatgat

SEQ ID NO: 155 - DNA, 18 bp, Synthetic construct
  source 1..18: mol_type = other DNA
agtattgctattcataaa

SEQ ID NO: 156 - DNA, 18 bp, Synthetic construct
  source 1..18: mol_type = other DNA
cttcagtattgctattca

SEQ ID NO: 157 - DNA, 18 bp, Synthetic construct
  source 1..18: mol_type = other DNA
atttcttcagtattgcta

SEQ ID NO: 158 - DNA, 18 bp, Synthetic construct
  source 1..18: mol_type = other DNA
tttaatttcttcagtatt

SEQ ID NO: 159 - DNA, 18 bp, Synthetic construct
  source 1..18: mol_type = other DNA
ttgttttaatttcttcag

SEQ ID NO: 160 - DNA, 18 bp, Synthetic construct
  source 1..18: mol_type = other DNA
tcttttgttttaatttct

SEQ ID NO: 161 - DNA, 18 bp, Synthetic construct
  source 1..18: mol_type = other DNA
gcaatcttttgttttaat

SEQ ID NO: 162 - DNA, 18 bp, Synthetic construct
  source 1..18: mol_type = other DNA
gacagcaatcttttgttt

SEQ ID NO: 163 - DNA, 18 bp, Synthetic construct
  source 1..18: mol_type = other DNA
ttgagacagcaatctttt

SEQ ID NO: 164 - DNA, 18 bp, Synthetic construct
  source 1..18: mol_type = other DNA
tatattgagacagcaatc

SEQ ID NO: 165 - DNA, 18 bp, Synthetic construct
  source 1..18: mol_type = other DNA
aagatatattgagacagc

SEQ ID NO: 166 - DNA, 18 bp, Synthetic construct
  source 1..18: mol_type = other DNA
gtaataaaatccccaggt

SEQ ID NO: 167 - DNA, 18 bp, Synthetic construct
  source 1..18: mol_type = other DNA
cagagtaataaaatcccc

SEQ ID NO: 168 - DNA, 18 bp, Synthetic construct
  source 1..18: mol_type = other DNA
ttcccagagtaataaaat

SEQ ID NO: 169 - DNA, 18 bp, Synthetic construct
  source 1..18: mol_type = other DNA
ataattcccagagtaata

SEQ ID NO: 170 - DNA, 18 bp, Synthetic construct
  source 1..18: mol_type = other DNA
acacataattcccagagt

SEQ ID NO: 171 - DNA, 18 bp, Synthetic construct
  source 1..18: mol_type = other DNA
cagaacacataattccca

SEQ ID NO: 172 - DNA, 18 bp, Synthetic construct
  source 1..18: mol_type = other DNA
ggggcagaacacataatt

SEQ ID NO: 173 - DNA, 18 bp, Synthetic construct
  source 1..18: mol_type = other DNA
tgatggggcagaacacat

SEQ ID NO: 174 - DNA, 18 bp, Synthetic construct
  source 1..18: mol_type = other DNA
agagtgatggggcagaac

SEQ ID NO: 175 - DNA, 18 bp, Synthetic construct
  source 1..18: mol_type = other DNA
agagagagtgatggggca

SEQ ID NO: 176 - DNA, 18 bp, Synthetic construct
  source 1..18: mol_type = other DNA
attaagagagagtgatgg

SEQ ID NO: 177 - DNA, 18 bp, Synthetic construct
  source 1..18: mol_type = other DNA
tccaattaagagagagtg

SEQ ID NO: 178 - DNA, 18 bp, Synthetic construct
  source 1..18: mol_type = other DNA
aaaatccaattaagagag

SEQ ID NO: 179 - DNA, 18 bp, Synthetic construct
  source 1..18: mol_type = other DNA
tttaaaaatccaattaag

SEQ ID NO: 180 - DNA, 18 bp, Synthetic construct
  source 1..18: mol_type = other DNA
taattttaaaaatccaat

SEQ ID NO: 181 - DNA, 18 bp, Synthetic construct
  source 1..18: mol_type = other DNA
aatataattttaaaaatc

SEQ ID NO: 182 - DNA, 18 bp, Synthetic construct
  source 1..18: mol_type = other DNA
tatgaatataattttaaa

SEQ ID NO: 183 - DNA, 18 bp, Synthetic construct
  source 1..18: mol_type = other DNA
gcaatatgaatataattt

SEQ ID NO: 184 - DNA, 18 bp, Synthetic construct
  source 1..18: mol_type = other DNA
tcctgcaatatgaatata

SEQ ID NO: 185 - DNA, 18 bp, Synthetic construct
  source 1..18: mol_type = other DNA
cgagtcctgcaatatgaa

SEQ ID NO: 186 - DNA, 18 bp, Synthetic construct
  source 1..18: mol_type = other DNA
ctgccgagtcctgcaata

SEQ ID NO: 187 - DNA, 18 bp, Synthetic construct
  source 1..18: mol_type = other DNA
tcttctgccgagtcctgc

SEQ ID NO: 188 - DNA, 18 bp, Synthetic construct
  source 1..18: mol_type = other DNA
aaggtcttctgccgagtc

SEQ ID NO: 189 - DNA, 18 bp, Synthetic construct
  source 1..18: mol_type = other DNA
ctcgaaggtcttctgccg

SEQ ID NO: 190 - DNA, 18 bp, Synthetic construct
  source 1..18: mol_type = other DNA
ttctctcgaaggtcttct

SEQ ID NO: 191 - DNA, 18 bp, Synthetic construct
  source 1..18: mol_type = other DNA
acctttctctcgaaggtc

SEQ ID NO: 192 - DNA, 18 bp, Synthetic construct
  source 1..18: mol_type = other DNA
ttctacctttctctcgaa

SEQ ID NO: 193 - DNA, 18 bp, Synthetic construct
  source 1..18: mol_type = other DNA
tattttctacctttctct

SEQ ID NO: 194 - DNA, 18 bp, Synthetic construct
  source 1..18: mol_type = other DNA
ttcttattttctaccttt

SEQ ID NO: 195 - DNA, 18 bp, Synthetic construct
  source 1..18: mol_type = other DNA
caaattcttattttctac

SEQ ID NO: 196 - DNA, 18 bp, Synthetic construct
  source 1..18: mol_type = other DNA
gagccaaattcttatttt

SEQ ID NO: 197 - DNA, 18 bp, Synthetic construct
  source 1..18: mol_type = other DNA
cagagagccaaattctta

SEQ ID NO: 198 - DNA, 18 bp, Synthetic construct
  source 1..18: mol_type = other DNA
cacacagagagccaaatt

SEQ ID NO: 199 - DNA, 18 bp, Synthetic construct
  source 1..18: mol_type = other DNA
tgctcacacagagagcca

SEQ ID NO: 200 - DNA, 18 bp, Synthetic construct
  source 1..18: mol_type = other DNA
cacatgctcacacagaga

SEQ ID NO: 201 - DNA, 18 bp, Synthetic construct
  source 1..18: mol_type = other DNA
cgcacacatgctcacaca

SEQ ID NO: 202 - DNA, 18 bp, Synthetic construct
  source 1..18: mol_type = other DNA
cacacgcacacatgctca

SEQ ID NO: 203 - DNA, 18 bp, Synthetic construct
  source 1..18: mol_type = other DNA
cgcacacacgcacacatg

SEQ ID NO: 204 - DNA, 18 bp, Synthetic construct
  source 1..18: mol_type = other DNA
ctctcgcacacacgcaca

SEQ ID NO: 205 - DNA, 18 bp, Synthetic construct
  source 1..18: mol_type = other DNA
ctctctctcgcacacacg

SEQ ID NO: 206 - DNA, 18 bp, Synthetic construct
  source 1..18: mol_type = other DNA
gtctctctctctcgcaca

SEQ ID NO: 207 - DNA, 18 bp, Synthetic construct
  source 1..18: mol_type = other DNA
gtctgtctctctctctcg

SEQ ID NO: 208 - DNA, 18 bp, Synthetic construct
  source 1..18: mol_type = other DNA
ggctgtctgtctctctct

SEQ ID NO: 209 - DNA, 18 bp, Synthetic construct
  source 1..18: mol_type = other DNA
ggcaggctgtctgtctct

SEQ ID NO: 210 - DNA, 18 bp, Synthetic construct
  source 1..18: mol_type = other DNA
cttaggcaggctgtctgt

SEQ ID NO: 211 - DNA, 18 bp, Synthetic construct
  source 1..18: mol_type = other DNA
tcttcttaggcaggctgt

SEQ ID NO: 212 - DNA, 18 bp, Synthetic construct
  source 1..18: mol_type = other DNA
catttcttcttaggcagg

SEQ ID NO: 213 - DNA, 18 bp, Synthetic construct
  source 1..18: mol_type = other DNA
cattcatttcttcttagg

SEQ ID NO: 214 - DNA, 18 bp, Synthetic construct
  source 1..18: mol_type = other DNA
ttcacattcatttcttct

SEQ ID NO: 215 - DNA, 18 bp, Synthetic construct
  source 1..18: mol_type = other DNA
cgcattcacattcatttc

SEQ ID NO: 216 - DNA, 18 bp, Synthetic construct
  source 1..18: mol_type = other DNA
aagccgcattcacattca

SEQ ID NO: 217 - DNA, 18 bp, Synthetic construct
  source 1..18: mol_type = other DNA
ccacaagccgcattcaca

SEQ ID NO: 218 - DNA, 18 bp, Synthetic construct
  source 1..18: mol_type = other DNA
tgtgccacaagccgcatt

SEQ ID NO: 219 - DNA, 18 bp, Synthetic construct
  source 1..18: mol_type = other DNA
caactgtgccacaagccg

SEQ ID NO: 220 - DNA, 18 bp, Synthetic construct
  source 1..18: mol_type = other DNA
ttgtcaactgtgccacaa

SEQ ID NO: 221 - DNA, 18 bp, Synthetic construct
  source 1..18: mol_type = other DNA
atccttgtcaactgtgcc

SEQ ID NO: 222 - DNA, 18 bp, Synthetic construct
  source 1..18: mol_type = other DNA
tatcatccttgtcaactg

SEQ ID NO: 223 - DNA, 18 bp, Synthetic construct
  source 1..18: mol_type = other DNA
gatttatcatccttgtca

SEQ ID NO: 224 - DNA, 18 bp, Synthetic construct
  source 1..18: mol_type = other DNA
tattgatttatcatcctt

SEQ ID NO: 225 - DNA, 18 bp, Synthetic construct
  source 1..18: mol_type = other DNA
gcattattgatttatcat

SEQ ID NO: 226 - DNA, 18 bp, Synthetic construct
  source 1..18: mol_type = other DNA
gcttgcattattgattta

SEQ ID NO: 227 - DNA, 18 bp, Synthetic construct
  source 1..18: mol_type = other DNA
gtaagcttgcattattga

SEQ ID NO: 228 - DNA, 18 bp, Synthetic construct
  source 1..18: mol_type = other DNA
gatagtaagcttgcatta

SEQ ID NO: 229 - DNA, 18 bp, Synthetic construct
  source 1..18: mol_type = other DNA
aaatgatagtaagcttgc

SEQ ID NO: 230 - DNA, 18 bp, Synthetic construct
  source 1..18: mol_type = other DNA
tcataaatgatagtaagc

SEQ ID NO: 231 - DNA, 18 bp, Synthetic construct
  source 1..18: mol_type = other DNA
ctattcataaatgatagt

SEQ ID NO: 232 - DNA, 18 bp, Synthetic construct
  source 1..18: mol_type = other DNA
attgctattcataaatga

SEQ ID NO: 233 - DNA, 18 bp, Synthetic construct
  source 1..18: mol_type = other DNA
cagtattgctattcataa

SEQ ID NO: 234 - DNA, 18 bp, Synthetic construct
  source 1..18: mol_type = other DNA
tcttcagtattgctattc

SEQ ID NO: 235 - DNA, 18 bp, Synthetic construct
  source 1..18: mol_type = other DNA
aatttcttcagtattgct

SEQ ID NO: 236 - DNA, 18 bp, Synthetic construct
  source 1..18: mol_type = other DNA
ttttaatttcttcagtat

SEQ ID NO: 237 - DNA, 18 bp, Synthetic construct
  source 1..18: mol_type = other DNA
tttgttttaatttcttca

SEQ ID NO: 238 - DNA, 18 bp, Synthetic construct
  source 1..18: mol_type = other DNA
atcttttgttttaatttc

SEQ ID NO: 239 - DNA, 18 bp, Synthetic construct
  source 1..18: mol_type = other DNA
agcaatcttttgttttaa

SEQ ID NO: 240 - DNA, 18 bp, Synthetic construct
  source 1..18: mol_type = other DNA
agacagcaatcttttgtt

SEQ ID NO: 241 - DNA, 18 bp, Synthetic construct
  source 1..18: mol_type = other DNA
attgagacagcaatcttt

SEQ ID NO: 242 - DNA, 18 bp, Synthetic construct
  source 1..18: mol_type = other DNA
atatattgagacagcaat

SEQ ID NO: 243 - DNA, 18 bp, Synthetic construct
  source 1..18: mol_type = other DNA
taagatatattgagacag

SEQ ID NO: 244 - DNA, 18 bp, Synthetic construct
  source 1..18: mol_type = other DNA
agtaataaaatccccagg

SEQ ID NO: 245 - DNA, 18 bp, Synthetic construct
  source 1..18: mol_type = other DNA
ccagagtaataaaatccc

SEQ ID NO: 246 - DNA, 18 bp, Synthetic construct
  source 1..18: mol_type = other DNA
attcccagagtaataaaa

SEQ ID NO: 247 - DNA, 18 bp, Synthetic construct
  source 1..18: mol_type = other DNA
cataattcccagagtaat

SEQ ID NO: 248 - DNA, 18 bp, Synthetic construct
  source 1..18: mol_type = other DNA
aacacataattcccagag

SEQ ID NO: 249 - DNA, 18 bp, Synthetic construct
  source 1..18: mol_type = other DNA
gcagaacacataattccc

SEQ ID NO: 250 - DNA, 18 bp, Synthetic construct
  source 1..18: mol_type = other DNA
tggggcagaacacataat

SEQ ID NO: 251 - DNA, 18 bp, Synthetic construct
  source 1..18: mol_type = other DNA
gtgatggggcagaacaca

SEQ ID NO: 252 - DNA, 18 bp, Synthetic construct
  source 1..18: mol_type = other DNA
gagagtgatggggcagaa

SEQ ID NO: 253 - DNA, 18 bp, Synthetic construct
  source 1..18: mol_type = other DNA
aagagagagtgatggggc

SEQ ID NO: 254 - DNA, 18 bp, Synthetic construct
  source 1..18: mol_type = other DNA
aattaagagagagtgatg

SEQ ID NO: 255 - DNA, 18 bp, Synthetic construct
  source 1..18: mol_type = other DNA
atccaattaagagagagt

SEQ ID NO: 256 - DNA, 18 bp, Synthetic construct
  source 1..18: mol_type = other DNA
aaaaatccaattaagaga

SEQ ID NO: 257 - DNA, 18 bp, Synthetic construct
  source 1..18: mol_type = other DNA
ttttaaaaatccaattaa

SEQ ID NO: 258 - DNA, 18 bp, Synthetic construct
  source 1..18: mol_type = other DNA
ataattttaaaaatccaa

SEQ ID NO: 259 - DNA, 18 bp, Synthetic construct
  source 1..18: mol_type = other DNA
gaatataattttaaaaat

SEQ ID NO: 260 - DNA, 18 bp, Synthetic construct
  source 1..18: mol_type = other DNA
atatgaatataattttaa

SEQ ID NO: 261 - DNA, 18 bp, Synthetic construct
  source 1..18: mol_type = other DNA
tgcaatatgaatataatt

SEQ ID NO: 262 - DNA, 18 bp, Synthetic construct
  source 1..18: mol_type = other DNA
gtcctgcaatatgaatat

SEQ ID NO: 263 - DNA, 18 bp, Synthetic construct
  source 1..18: mol_type = other DNA
ccgagtcctgcaatatga

SEQ ID NO: 264 - DNA, 18 bp, Synthetic construct
  source 1..18: mol_type = other DNA
tctgccgagtcctgcaat

SEQ ID NO: 265 - DNA, 18 bp, Synthetic construct
  source 1..18: mol_type = other DNA
gtcttctgccgagtcctg

SEQ ID NO: 266 - DNA, 18 bp, Synthetic construct
  source 1..18: mol_type = other DNA
gaaggtcttctgccgagt

SEQ ID NO: 267 - DNA, 18 bp, Synthetic construct
  source 1..18: mol_type = other DNA
tctcgaaggtcttctgcc

SEQ ID NO: 268 - DNA, 18 bp, Synthetic construct
  source 1..18: mol_type = other DNA
tttctctcgaaggtcttc

SEQ ID NO: 269 - DNA, 18 bp, Synthetic construct
  source 1..18: mol_type = other DNA
tacctttctctcgaaggt

SEQ ID NO: 270 - DNA, 18 bp, Synthetic construct
  source 1..18: mol_type = other DNA
tttctacctttctctcga

SEQ ID NO: 271 - DNA, 18 bp, Synthetic construct
  source 1..18: mol_type = other DNA
ttattttctacctttctc

SEQ ID NO: 272 - DNA, 18 bp, Synthetic construct
  source 1..18: mol_type = other DNA
attcttattttctacctt

SEQ ID NO: 273 - DNA, 18 bp, Synthetic construct
  source 1..18: mol_type = other DNA
ccaaattcttattttcta

SEQ ID NO: 274 - DNA, 18 bp, Synthetic construct
  source 1..18: mol_type = other DNA
agagccaaattcttattt

SEQ ID NO: 275 - DNA, 18 bp, Synthetic construct
  source 1..18: mol_type = other DNA
acagagagccaaattctt

SEQ ID NO: 276 - DNA, 18 bp, Synthetic construct
  source 1..18: mol_type = other DNA
tcacacagagagccaaat

SEQ ID NO: 277 - DNA, 18 bp, Synthetic construct
  source 1..18: mol_type = other DNA
atgctcacacagagagcc

SEQ ID NO: 278 - DNA, 18 bp, Synthetic construct
  source 1..18: mol_type = other DNA
acacatgctcacacagag

SEQ ID NO: 279 - DNA, 18 bp, Synthetic construct
  source 1..18: mol_type = other DNA
acgcacacatgctcacac

SEQ ID NO: 280 - DNA, 18 bp, Synthetic construct
  source 1..18: mol_type = other DNA
acacacgcacacatgctc

SEQ ID NO: 281 - DNA, 18 bp, Synthetic construct
  source 1..18: mol_type = other DNA
tcgcacacacgcacacat

SEQ ID NO: 282 - DNA, 18 bp, Synthetic construct
  source 1..18: mol_type = other DNA
tctctcgcacacacgcac

SEQ ID NO: 283 - DNA, 18 bp, Synthetic construct
  source 1..18: mol_type = other DNA
tctctctctcgcacacac

SEQ ID NO: 284 - DNA, 18 bp, Synthetic construct
  source 1..18: mol_type = other DNA
tgtctctctctctcgcac

SEQ ID NO: 285 - DNA, 18 bp, Synthetic construct
  source 1..18: mol_type = other DNA
tgtctgtctctctctctc

SEQ ID NO: 286 - DNA, 18 bp, Synthetic construct
  source 1..18: mol_type = other DNA
aggctgtctgtctctctc

SEQ ID NO: 287 - DNA, 18 bp, Synthetic construct
  source 1..18: mol_type = other DNA
aggcaggctgtctgtctc

SEQ ID NO: 288 - DNA, 18 bp, Synthetic construct
  source 1..18: mol_type = other DNA
tcttaggcaggctgtctg

SEQ ID NO: 289 - DNA, 18 bp, Synthetic construct
  source 1..18: mol_type = other DNA
ttcttcttaggcaggctg

SEQ ID NO: 290 - DNA, 18 bp, Synthetic construct
  source 1..18: mol_type = other DNA
tcatttcttcttaggcag

SEQ ID NO: 291 - DNA, 18 bp, Synthetic construct
  source 1..18: mol_type = other DNA
acattcatttcttcttag

SEQ ID NO: 292 - DNA, 18 bp, Synthetic construct
  source 1..18: mol_type = other DNA
attcacattcatttcttc

SEQ ID NO: 293 - DNA, 18 bp, Synthetic construct
  source 1..18: mol_type = other DNA
ccgcattcacattcattt

SEQ ID NO: 294 - DNA, 18 bp, Synthetic construct
  source 1..18: mol_type = other DNA
caagccgcattcacattc

SEQ ID NO: 295 - DNA, 18 bp, Synthetic construct
  source 1..18: mol_type = other DNA
gccacaagccgcattcac

SEQ ID NO: 296 - DNA, 18 bp, Synthetic construct
  source 1..18: mol_type = other DNA
ctgtgccacaagccgcat

SEQ ID NO: 297 - DNA, 18 bp, Synthetic construct
  source 1..18: mol_type = other DNA
tcaactgtgccacaagcc

SEQ ID NO: 298 - DNA, 18 bp, Synthetic construct
  source 1..18: mol_type = other DNA
cttgtcaactgtgccaca

SEQ ID NO: 299 - DNA, 18 bp, Synthetic construct
  source 1..18: mol_type = other DNA
catccttgtcaactgtgc

SEQ ID NO: 300 - DNA, 18 bp, Synthetic construct
  source 1..18: mol_type = other DNA
ttatcatccttgtcaact

SEQ ID NO: 301 - DNA, 18 bp, Synthetic construct
  source 1..18: mol_type = other DNA
tgatttatcatccttgtc

SEQ ID NO: 302 - DNA, 18 bp, Synthetic construct
  source 1..18: mol_type = other DNA
ttattgatttatcatcct

SEQ ID NO: 303 - DNA, 18 bp, Synthetic construct
  source 1..18: mol_type = other DNA
tgcattattgatttatca

SEQ ID NO: 304 - DNA, 18 bp, Synthetic construct
  source 1..18: mol_type = other DNA
agcttgcattattgattt

SEQ ID NO: 305 - DNA, 18 bp, Synthetic construct
  source 1..18: mol_type = other DNA
agtaagcttgcattattg

SEQ ID NO: 306 - DNA, 18 bp, Synthetic construct
  source 1..18: mol_type = other DNA
tgatagtaagcttgcatt

SEQ ID NO: 307 - DNA, 18 bp, Synthetic construct
  source 1..18: mol_type = other DNA
taaatgatagtaagcttg

SEQ ID NO: 308 - DNA, 18 bp, Synthetic construct
  source 1..18: mol_type = other DNA
ttcataaatgatagtaag

SEQ ID NO: 309 - DNA, 18 bp, Synthetic construct
  source 1..18: mol_type = other DNA
gctattcataaatgatag

SEQ ID NO: 310 - DNA, 18 bp, Synthetic construct
  source 1..18: mol_type = other DNA
tattgctattcataaatg

SEQ ID NO: 311 - DNA, 18 bp, Synthetic construct
  source 1..18: mol_type = other DNA
tcagtattgctattcata

SEQ ID NO: 312 - DNA, 18 bp, Synthetic construct
  source 1..18: mol_type = other DNA
ttcttcagtattgctatt

SEQ ID NO: 313 - DNA, 18 bp, Synthetic construct
  source 1..18: mol_type = other DNA
taatttcttcagtattgc

SEQ ID NO: 314 - DNA, 18 bp, Synthetic construct
  source 1..18: mol_type = other DNA
gttttaatttcttcagta

SEQ ID NO: 315 - DNA, 18 bp, Synthetic construct
  source 1..18: mol_type = other DNA
ttttgttttaatttcttc

SEQ ID NO: 316 - DNA, 18 bp, Synthetic construct
  source 1..18: mol_type = other DNA
aatcttttgttttaattt

SEQ ID NO: 317 - DNA, 18 bp, Synthetic construct
  source 1..18: mol_type = other DNA
cagcaatcttttgtttta

SEQ ID NO: 318 - DNA, 18 bp, Synthetic construct
  source 1..18: mol_type = other DNA
gagacagcaatcttttgt

SEQ ID NO: 319 - DNA, 18 bp, Synthetic construct
  source 1..18: mol_type = other DNA
tattgagacagcaatctt

SEQ ID NO: 320 - DNA, 18 bp, Synthetic construct
  source 1..18: mol_type = other DNA
gatatattgagacagcaa

SEQ ID NO: 321 - DNA, 18 bp, Synthetic construct
  source 1..18: mol_type = other DNA
ataagatatattgagaca

(19) United States
(12) Patent Application Publication
(10) Pub. No.: US 2024/0316092 A1
(43) Pub. Date: Sep. 26, 2024

(54) COMPOUNDS AND METHODS FOR INCREASING STMN2 EXPRESSION

(71) Applicants: Ionis Pharmaceuticals, Inc., Carlsbad, CA (US); Ludwig Institute For Cancer Research, Zurich (CH)

(72) Inventors: Huynh-Hoa Bui, San Diego, CA (US); Don W. Cleveland, La Jolla, CA (US); Ze'ev Melamed, San Diego, CA (US)

(73) Assignees: Ionis Pharmaceuticals, Inc., Carlsbad, CA (US); Ludwig Institute For Cancer Research, Zurich (CH)

(21) Appl. No.: 18/493,056

(22) Filed: Oct. 24, 2023

Related U.S. Application Data
(63) Continuation of application No. 16/973,652, filed on Dec. 9, 2020, now Pat. No. 11,833,168.
(60) Provisional application No. 62/685,192, filed on Jun. 14, 2018.

(51) Int. Cl. A61K 31/7125; C12N 15/113

(52) U.S. Cl. CPC A61K 31/7125; C12N 15/113; C12N 2310/315; C12N 2310/3181; C12N 2310/321; C12N 2310/3341; C12N 2310/341; C12N 2310/344; C12N 2310/346; C12N 2310/351

(57) ABSTRACT

Provided are compounds, methods, and pharmaceutical compositions for increasing the amount or activity of STMN2 RNA in a cell or animal, and in certain embodiments increasing the amount of STMN2 protein in a cell or animal. Such compounds, methods, and pharmaceutical compositions are useful to ameliorate at least one symptom of a neurodegenerative disease. Such symptoms include ataxia, neuropathy, synaptic dysfunction, deficits in cognition, and decreased longevity. Such neurodegenerative diseases include amyotrophic lateral sclerosis (ALS), frontotemporal dementia (FTD), Alzheimer's disease (AD), and dementia with Lewy bodies (DLB).

### SEQUENCE LISTING

The present application is being filed along with a Sequence Listing in electronic format. The Sequence Listing is provided as a file entitled BIOL0338SEQ.xml, created on Oct. 18, 2023, which is 336 KB in size. The information in the electronic format of the sequence listing is incorporated herein by reference in its entirety.

### FIELD

Provided are compounds, methods, and pharmaceutical compositions for increasing the amount or activity of STMN2 RNA in a cell or animal, and in certain instances increasing the amount of stathmin-2 protein in a cell or animal. Such compounds, methods, and pharmaceutical compositions are useful to ameliorate at least one symptom of a neurodegenerative disease. Such symptoms include ataxia, neuropathy, synaptic dysfunction, deficits in cognition, and decreased longevity. Such neurodegenerative diseases include amyotrophic lateral sclerosis (ALS), frontotemporal dementia (FTD), Alzheimer's disease (AD), and dementia with Lewy bodies (DLB).

### BACKGROUND

The STMN2 gene encodes the stathmin-2 protein, a member of the stathmin family of phosphoproteins. Stathmin proteins function in microtubule dynamics and signal transduction. Stathmin-2 plays a regulatory role in neuronal growth and is also thought to be involved in osteogenesis.

Amyotrophic lateral sclerosis (ALS, also known as Lou Gehrig's disease) is a disorder characterized by a selective degeneration of upper and lower motor neurons (Rowland, N. Engl. J. Med. 2001, 344, 1688-1700). ALS is a devastating progressive neurodegenerative disease affecting as many as 30,000 Americans at any given time. The progressive degeneration of the motor neurons in ALS eventually leads to their death. When the motor neurons die, the ability of the brain to initiate and control muscle movement is lost. With voluntary muscle action progressively affected, patients in the later stages of the disease may become totally paralyzed.

Frontotemporal dementia (FTD) refers to a group of disorders caused by progressive nerve cell loss in the brain's frontal lobes or temporal lobes. Nerve cell damage caused by FTD leads to loss of function in the frontal lobes or temporal lobes, which variably cause deterioration in behavior and personality, language disturbances, or alterations in muscle or motor functions.

Alzheimer's disease (AD) is an irreversible, progressive brain disorder that slowly destroys memory and thinking skills, and eventually the ability to carry out the simplest tasks. AD is the most common cause of dementia among older adults. Dementia is the loss of cognitive functioning and behavioral abilities to such an extent that it interferes with a person's daily life and activities. Dementia ranges in severity from the mildest stage, when it is just beginning to affect a person's functioning, to the most severe stage, when the person must depend completely on others for basic activities of daily living.

Dementia with Lewy bodies (DLB) is a type of progressive dementia that leads to a decline in thinking, reasoning and independent function because of abnormal Lewy body deposition in neurons. DLB causes a progressive decline in mental abilities. People with DLB may experience visual hallucinations, and changes in alertness and attention. Other effects include Parkinson's disease-like symptoms such as rigid muscles, slow movement, and tremors.

Currently there is a lack of acceptable options for treating neurodegenerative diseases such as amyotrophic lateral sclerosis (ALS), frontotemporal dementia (FTD), Alzheimer's disease (AD), and dementia with Lewy bodies (DLB). It is therefore an object herein to provide compounds, methods, and pharmaceutical compositions for the treatment of such diseases.

### SUMMARY OF THE INVENTION

Provided herein are compounds, methods, and pharmaceutical compositions for increasing the amount or activity of STMN2 RNA, and in certain embodiments increasing the amount of stathmin-2 protein in a cell or animal. While not limited to a particular mechanism, it is thought that the modified oligonucleotides described herein increase STMN2 RNA expression by preventing usage of a premature polyadenylation site in the first intron of STMN2 pre-mRNA. In certain embodiments, the animal has a neurodegenerative disease. In certain embodiments, the animal has amyotrophic lateral sclerosis (ALS), frontotemporal dementia (FTD), Alzheimer's disease (AD), and dementia with Lewy bodies (DLB). In certain embodiments, compounds useful for increasing expression of STMN2 RNA are oligomeric compounds or modified oligonucleotides. In certain embodiments, the oligomeric compound comprises a modified oligonucleotide.

Also provided are methods useful for ameliorating at least one symptom of a neurodegenerative disease. In certain embodiments, the neurodegenerative disease is amyotrophic lateral sclerosis (ALS), frontotemporal dementia (FTD), Alzheimer's disease (AD), and dementia with Lewy bodies (DLB). In certain embodiments symptoms include ataxia, neuropathy, synaptic dysfunction, deficits in cognition, and decreased longevity. In certain embodiments, amelioration of these symptoms results in improved motor function, reduced neuropathy, improved synaptic function, improved cognition, and survival.

### DETAILED DESCRIPTION OF THE INVENTION

It is to be understood that both the foregoing general description and the following detailed description are exemplary and explanatory only and are not restrictive. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated-by-reference for the portions of the document discussed herein, as well as in their entirety.

### DEFINITIONS

Unless specific definitions are provided, the nomenclature used in connection with, and the procedures and techniques of, analytical chemistry, synthetic organic chemistry, and medicinal and pharmaceutical chemistry described herein are those well known and commonly used in the art. Where permitted, all patents, applications, published applications and other publications and other data referred to throughout in the disclosure are incorporated by reference herein in their entirety.

Unless otherwise indicated, the following terms have the following meanings:

### DEFINITIONS

As used herein, “2′-deoxynucleoside” means a nucleoside comprising a 2′-H(H) deoxyribosyl sugar moiety, as found in naturally occurring deoxyribonucleic acids (DNA). In certain embodiments, a 2′-deoxynucleoside may comprise a modified nucleobase or may comprise an RNA nucleobase (uracil).

As used herein, “2′-substituted nucleoside” means a nucleoside comprising a 2′-substituted sugar moiety. As used herein, “2′-substituted” in reference to a sugar moiety means a sugar moiety comprising at least one 2′-substituent group other than H or OH.

As used herein, “5-methyl cytosine” means a cytosine modified with a methyl group attached to the 5-position. A 5-methyl cytosine is a modified nucleobase.

As used herein, “administering” means providing a pharmaceutical agent to an animal.

As used herein, “animal” means a human or non-human animal.

As used herein, “antisense activity” means any detectable and/or measurable change attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound.

As used herein, “antisense compound” means an oligomeric compound or oligomeric duplex capable of achieving at least one antisense activity.

As used herein, “ameliorate” in reference to a treatment means improvement in at least one symptom relative to the same symptom in the absence of the treatment. In certain embodiments, amelioration is the reduction in the severity or frequency of a symptom or the delayed onset or slowing of progression in the severity or frequency of a symptom. In certain embodiments, the symptom is ataxia, neuropathy, synaptic dysfunction, deficits in cognition, and decreased longevity. In certain embodiments, amelioration of these symptoms results in improved motor function, reduced neuropathy, improved synaptic function, improved cognition, and survival.

As used herein, “bicyclic nucleoside” or “BNA” means a nucleoside comprising a bicyclic sugar moiety. As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety. In certain embodiments, the bicyclic sugar moiety does not comprise a furanosyl moiety.

As used herein, “cleavable moiety” means a bond or group of atoms that is cleaved under physiological conditions, for example, inside a cell, an animal, or a human.

As used herein, “complementary” in reference to an oligonucleotide means that at least 70% of the nucleobases of the oligonucleotide or one or more regions thereof and the nucleobases of another nucleic acid or one or more regions thereof are capable of hydrogen bonding with one another when the nucleobase sequence of the oligonucleotide and the other nucleic acid are aligned in opposing directions. Complementary nucleobases means nucleobases that are capable of forming hydrogen bonds with one another. Complementary nucleobase pairs include adenine (A) and thymine (T), adenine (A) and uracil (U), cytosine (C) and guanine (G), 5-methyl cytosine (mC) and guanine (G). Complementary oligonucleotides and/or nucleic acids need not have nucleobase complementarity at each nucleoside. Rather, some mismatches are tolerated. As used herein, “fully complementary” or “100% complementary” in reference to oligonucleotides means that oligonucleotides are complementary to another oligonucleotide or nucleic acid at each nucleoside of the oligonucleotide.

As used herein, “conjugate group” means a group of atoms that is directly or indirectly attached to an oligonucleotide. Conjugate groups include a conjugate moiety and a conjugate linker that attaches the conjugate moiety to the oligonucleotide.

As used herein, “conjugate linker” means a single bond or a group of atoms comprising at least one bond that connects a conjugate moiety to an oligonucleotide.

As used herein, “conjugate moiety” means a group of atoms that is attached to an oligonucleotide via a conjugate linker.

As used herein, “contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

As used herein, “constrained ethyl” or “cEt” or “cEt modified sugar” means a β-D ribosyl bicyclic sugar moiety wherein the second ring of the bicyclic sugar is formed via a bridge connecting the 4′-carbon and the 2′carbon of the β-D ribosyl sugar moiety, wherein the bridge has the formula 4′-CH(CH₃)—O-2′, and wherein the methyl group of the bridge is in the S configuration.

As used herein, “cEt” nucleoside” means a nucleoside comprising a cEt modified sugar.

As used herein, “chirally enriched population” means a plurality of molecules of identical molecular formula, wherein the number or percentage of molecules within the population that contain a particular stereochemical configuration at a particular chiral center is greater than the number or percentage of molecules expected to contain the same particular stereochemical configuration at the same particular chiral center within the population if the particular chiral center were stereorandom. Chiraly enriched populations of molecules having multiple chiral centers within each molecule may contain one or more stereorandom chiral centers. In certain embodiments, the molecules are modified oligonucleotides. In certain embodiments, the molecules are compounds comprising modified oligonucleotides.

As used herein, “gapmer” means a modified oligonucleotide comprising an internal region having a plurality of nucleosides that support RNase H cleavage positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings.” Unless otherwise indicated. “gapmer” refers to a sugar motif. Unless otherwise indicated, the sugar moieties of the nucleosides of the gap of a gapmer are unmodified 2′-deoxyribosyl. Thus, the term “MOE gapmer” indicates a gapmer having a sugar motif of 2′-MOE nucleosides in both wings and a gap of 2′-deoxynucleosides. Unless otherwise indicated, a MOE gapmer may comprise one or more modified internucleoside linkages and/or modified nucleobases and such modifications do not necessarily follow the gapmer pattern of the sugar modifications.

As used herein, “hotspot region” is a range of nucleobases on a target nucleic acid amenable to oligomeric compound-mediated increase of the amount or activity of the target nucleic acid.

As used herein, “hybridization” means the pairing or annealing of complementary oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases.

As used herein, “increasing the amount or activity” refers to more transcriptional expression or activity relative to the transcriptional expression or activity in an untreated or control sample.

As used herein, the term “internucleoside linkage” is the covalent linkage between adjacent nucleosides in an oligonucleotide. As used herein “modified internucleoside linkage” means any internucleoside linkage other than a phosphodiester internucleoside linkage. “Phosphorothioate linkage” is a modified internucleoside linkage in which one of the non-bridging oxygen atoms of a phosphodiester internucleoside linkage is replaced with a sulfur atom.

As used herein, “linker-nucleoside” means a nucleoside that links, either directly or indirectly, an oligonucleotide to a conjugate moiety. Linker-nucleosides are located within the conjugate linker of an oligomeric compound. Linker-nucleosides are not considered part of the oligonucleotide portion of an oligomeric compound even if they are contiguous with the oligonucleotide.

As used herein, “non-bicyclic modified sugar moiety” means a modified sugar moiety that comprises a modification, such as a substituent, that does not form a bridge between two atoms of the sugar to form a second ring.

As used herein, “mismatch” or “non-complementary” means a nucleobase of a first oligonucleotide that is not complementary with the corresponding nucleobase of a second oligonucleotide or target nucleic acid when the first and second oligonucleotide are aligned.

As used herein, “MOE” means methoxyethyl. “2′-MOE” or “2′-MOE modified sugar” means a 2′-OCH₂CH₂OCH₃ group in place of the 2′ OH group of a ribosyl sugar moiety.

As used herein, “2′-MOE nucleoside” means a nucleoside comprising a 2′-MOE modified sugar.

As used herein, “motif” means the pattern of unmodified and/or modified sugar moieties, nucleobases, and/or internucleoside linkages, in an oligonucleotide.

As used herein, “neurodegenerative disease” means a condition marked by progressive loss of structure or function of neurons, including death of neurons. In certain embodiments, neurodegenerative disease is amyotrophic lateral sclerosis (ALS), frontotemporal dementia (FTD), Alzheimer's disease (AD), and dementia with Lewy bodies (DLB).

As used herein, “nucleobase” means an unmodified nucleobase or a modified nucleobase. As used herein an “unmodified nucleobase” is adenine (A), thymine (T), cytosine (C), uracil (U), and guanine (G). As used herein, a “modified nucleobase” is a group of atoms other than unmodified A, T, C, U, or G capable of pairing with at least one unmodified nucleobase. A “5-methyl cytosine” is a modified nucleobase. A universal base is a modified nucleobase that can pair with any one of the five unmodified nucleobases. As used herein, “nucleobase sequence” means the order of contiguous nucleobases in a nucleic acid or oligonucleotide independent of any sugar or internucleoside linkage modification.

As used herein, “nucleoside” means a compound comprising a nucleobase and a sugar moiety. The nucleobase and sugar moiety are each, independently, unmodified or modified. As used herein, “modified nucleoside” means a nucleoside comprising a modified nucleobase and/or a modified sugar moiety. Modified nucleosides include abasic nucleosides, which lack a nucleobase. “Linked nucleosides” are nucleosides that are connected in a contiguous sequence (i.e., no additional nucleosides are presented between those that are linked).

As used herein, “oligomeric compound” means an oligonucleotide and optionally one or more additional features, such as a conjugate group or terminal group. An oligomeric compound may be paired with a second oligomeric compound that is complementary to the first oligomeric compound or may be unpaired. A “singled-stranded oligomeric compound” is an unpaired oligomeric compound. The term “oligomeric duplex” means a duplex formed by two oligomeric compounds having complementary nucleobase sequences. Each oligomeric compound of an oligomeric duplex may be referred to as a “duplexed oligomeric compound.”

As used herein, “oligonucleotide” means a strand of linked nucleosides connected via internucleoside linkages, wherein each nucleoside and internucleoside linkage may be modified or unmodified. Unless otherwise indicated, oligonucleotides consist of 8-50 linked nucleosides. As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

As used herein, “pharmaceutically acceptable carrier or diluent” means any substance suitable for use in administering to an animal. Certain such carriers enable pharmaceutical compositions to be formulated as, for example, tablets, pills, dragees, capsules, liquids, gels, syrups, slurries, suspension and lozenges for the oral ingestion by a subject. In certain embodiments, a pharmaceutically acceptable carrier or diluent is sterile water, sterile saline, sterile buffer solution, or sterile artificial cerebrospinal fluid.

As used herein “pharmaceutically acceptable salts” means physiologically and pharmaceutically acceptable salts of compounds. Pharmaceutically acceptable salts retain the desired biological activity of the parent compound and do not impart undesired toxicological effects thereto.

As used herein “pharmaceutical composition” means a mixture of substances suitable for administering to a subject. For example, a pharmaceutical composition may comprise an oligomeric compound and a sterile aqueous solution. In certain embodiments, a pharmaceutical composition shows activity in free uptake assay in certain cell lines.

As used herein, “phosphorus moiety” means a group of atoms comprising a phosphorus atom. In certain embodiments, a phosphorus moiety comprises a mono-, di-, or tri-phosphate, or phosphorothioate.

As used herein “prodrug” means a therapeutic agent in a form outside the body that is converted to a different form within an animal or cells thereof. Typically, conversion of a prodrug within the animal is facilitated by the action of an enzyme (e.g., endogenous or viral enzyme) or chemicals present in cells or tissues and/or by physiologic conditions.

As used herein, “RNAi compound” means an antisense compound that acts, at least in part, through RISC or Ago2 to modulate a target nucleic acid and/or protein encoded by a target nucleic acid. RNAi compounds include, but are not limited to double-stranded siRNA, single-stranded RNA (ssRNA), and microRNA, including microRNA mimics. In certain embodiments, an RNAi compound modulates the amount, activity, and/or splicing of a target nucleic acid. The term RNAi compound excludes antisense compounds that act through RNase H.

As used herein, “self-complementary” in reference to an oligonucleotide means an oligonucleotide that at least partially hybridizes to itself.

As used herein, “standard cell assay” means the assay described in Example 1 and reasonable variations thereof.

As used herein, “stereorandom chiral center” in the context of a population of molecules of identical molecular formula means a chiral center having a random stereochemical configuration. For example, in a population of molecules comprising a stereorandom chiral center, the number of molecules having the (S) configuration of the stereorandom chiral center may be but is not necessarily the same as the number of molecules having the (R) configuration of the stereorandom chiral center. The stereochemical configuration of a chiral center is considered random when it is the result of a synthetic method that is not designed to control the stereochemical configuration. In certain embodiments, a stereorandom chiral center is a stereorandom phosphorothioate internucleoside linkage.

As used herein, “sugar moiety” means an unmodified sugar moiety or a modified sugar moiety. As used herein, “unmodified sugar moiety” means a 2′-OH (H) furanosyl moiety, as found in RNA (an “unmodified RNA sugar moiety”), or a 2′-H(H) moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. As used herein, “modified sugar moiety” or “modified sugar” means a modified furanosyl sugar moiety or a sugar surrogate.

As used herein, “sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or nucleic acids.

As used herein, “target nucleic acid” and “target RNA” mean a nucleic acid that an antisense compound is designed to affect.

As used herein, “target region” means a portion of a target nucleic acid to which an oligomeric compound is designed to hybridize.

As used herein, “terminal group” means a chemical group or group of atoms that is covalently linked to a terminus of an oligonucleotide.

As used herein, “therapeutically effective amount” means an amount of a pharmaceutical agent that provides a therapeutic benefit to an animal. For example, a therapeutically effective amount improves a symptom of a disease.

The present disclosure provides the following non-limiting numbered embodiments:
Embodiment 1. An oligomeric compound, comprising a modified oligonucleotide consisting of 12 to 50 linked nucleosides wherein the nucleobase sequence of the modified oligonucleotide is at least 90% complementary to an equal length portion of a STMN2 nucleic acid, and wherein the modified oligonucleotide comprises at least one modification selected from a modified sugar, a sugar surrogate, and a modified internucleoside linkage.
Embodiment 2. An oligomeric compound, comprising a modified oligonucleotide consisting of 12 to 50 linked nucleosides and having a nucleobase sequence comprising at least 8, at least 9, at least 10, at least 11, at least 12, at least 13, at least 14, at least 15, at least 16, at least 17, or at least 18 consecutive nucleobases of any of the nucleobase sequences of SEQ ID NOs: 10-321.
Embodiment 3. An oligomeric compound, comprising a modified oligonucleotide consisting of 12 to 50 linked nucleosides and having a nucleobase sequence comprising at least 8, at least 9, at least 10, at least 11, at least 12, at least 13, at least 14, at least 15, at least 16, at least 17, or at least 18 consecutive nucleobases complementary to:
8819-8841 of SEQ ID NO: 1 or 100-122 of SEQ ID NO: 2;
8827-8851 of SEQ ID NO: 1 or 108-132 of SEQ ID NO: 2;
8836-8880 of SEQ ID NO: 1 or 117-161 of SEQ ID NO 2; or
8913-8948 of SEQ ID NO: 1 or 194-229 of SEQ ID NO 2.
Embodiment 4. The oligomeric compound of any one of embodiments 1-3, wherein the STMN2 nucleic acid has the nucleobase sequence of SEQ ID NOs: 1 or SEQ ID NO: 2.
Embodiment 5. The oligomeric compound of any one of embodiments 1-4, wherein the modified oligonucleotide is a single-stranded modified oligonucleotide.
Embodiment 6. The oligomeric compound of any one of embodiments 1-5, wherein at least one internucleoside linkage of the modified oligonucleotide is a modified internucleoside linkage.
Embodiment 7. The oligomeric compound of embodiment 6, wherein the modified internucleoside linkage is a phosphorothioate internucleoside linkage.
Embodiment 8. The oligomeric compound of any one of embodiments 1-5, wherein each internucleoside linkage of the modified oligonucleotide is a modified internucleoside linkage.
Embodiment 9. The oligomeric compound of embodiment 8, wherein each modified internucleoside linkage of the modified oligonucleotide is a phosphorothioate internucleoside linkage.
Embodiment 10. The oligomeric compound of any one of embodiments 1-7, wherein at least one internucleoside linkage of the modified oligonucleotide is a phosphodiester internucleoside linkage.
Embodiment 11. The oligomeric compound of any one of embodiments 1-7 and 10, wherein each internucleoside linkage of the modified oligonucleotide is either a phosphodiester internucleoside linkage or a phosphorothioate internucleoside linkage.
Embodiment 12. The oligomeric compound of any one of embodiments 1-11, wherein at least one nucleobase of the modified oligonucleotide comprises a modified nucleobase.
Embodiment 13. The oligomeric compound of embodiment 12, wherein the modified nucleobase is a 5-methyl cytosine.
Embodiment 14. The oligomeric compound of any one of embodiments 1-13, wherein the modified oligonucleotide comprises at least 1, at least 2, at least 3, at least 4, at least 5, at least 6, at least 7, at least 8, at least 9, at least 10, at least 11, at least 12, at least 13, at least 14, at least 15, at least 16, at least 17, or at least 18 modified nucleosides comprising a modified sugar moiety.
Embodiment 15. The oligomeric compound of embodiment 14, wherein the modified oligonucleotide comprises at least 1, at least 2, at least 3, at least 4, at least 5, at least 6, at least 7, at least 8, at least 9, at least 10, at least 11, at least 12, at least 13, at least 14, at least 15, at least 16, at least 17, or at least 18 modified nucleosides comprising a bicyclic sugar moiety.
Embodiment 16. The oligomeric compound of embodiment 15, wherein the modified oligonucleotide comprises at least 1 modified nucleoside comprising a bicyclic sugar moiety having a 2′-4″ bridge, wherein the 2′-4′ bridge is selected from —O—CH₂—; and —O—CH(CH₃)—.
Embodiment 17. The oligomeric compound of any of embodiments 1-13, wherein the modified oligonucleotide comprises at least 1 modified nucleoside comprising a non-bicyclic sugar moiety.
Embodiment 18. The oligomeric compound of embodiment 17, wherein the modified oligonucleotide comprises at least 1 modified nucleoside comprising a modified non-bicyclic sugar moiety comprising a 2′-MOE or 2′-OMe.
Embodiment 19. The oligomeric compound of embodiment 18, wherein each modified nucleoside of the modified oligonucleotide comprises a modified non-bicyclic sugar moiety comprising a 2′-MOE or 2′-OMe.
Embodiment 20. The oligomeric compound of any of embodiments 1-13, wherein the modified oligonucleotide comprises at least 1 modified nucleoside comprising a sugar surrogate.
Embodiment 21. The oligomeric compound of embodiment 20, wherein the modified oligonucleotide comprises at least 1 modified nucleoside comprising a sugar surrogate selected from morpholino and PNA.
Embodiment 22. The oligomeric compound of any of embodiments 1-18 and 20-21, wherein the modified oligonucleotide is a gapmer.
Embodiment 23. The oligomeric compound of any of embodiments 1-18 and 20-21, wherein the modified oligonucleotide has a sugar motif comprising:
a 5′-region consisting of 1-6 linked 5′-nucleosides;
a central region consisting of 6-10 linked central region nucleosides; and
a 3′-region consisting of 1-6 linked 5′-nucleosides; wherein each of the 5′-region nucleosides and each of the 3′-region nucleosides comprises a modified sugar moiety and each of the central region nucleosides comprises a 2′-deoxynucleoside sugar moiety.
Embodiment 24. The oligomeric compound of embodiments 1-7 or 10-23, wherein the modified oligonucleotide consists of 20 linked nucleosides and has the following internucleoside motif: sooosssssssssssooss; wherein,
s=a phosphorothioate internucleoside linkage, and
o=a phosphodiester internucleoside linkage.
Embodiment 25. The oligomeric compound of any one of embodiments 1-23, wherein the modified oligonucleotide consists of 12-18, 12-20, 14-20, 16-20, or 17-19 linked nucleosides.
Embodiment 26. The oligomeric compound of any one of embodiments 1-23 and 25, wherein the modified oligonucleotide consists of 16, 17, or 18 linked nucleosides.
Embodiment 27. The oligomeric compound of any of embodiments 1-26 consisting of the modified oligonucleotide.
Embodiment 28. The oligomeric compound of any of embodiments 1-26 comprising a conjugate group comprising a conjugate moiety and a conjugate linker.
Embodiment 29. The oligomeric compound of embodiment 28, wherein the conjugate group comprises a GalNAc cluster comprising 1-3 GalNAc ligands.
Embodiment 30. The oligomeric compound of embodiment 28 or 29, wherein the conjugate linker consists of a single bond.

Embodiment 31. The oligomeric compound of embodiment 28, wherein the conjugate linker is cleavable.
Embodiment 32. The oligomeric compound of embodiment 28, wherein the conjugate linker comprises 1-3 linker-nucleosides.
Embodiment 33. The oligomeric compound of any of embodiments 28-32, wherein the conjugate group is attached to the modified oligonucleotide at the 5′-end of the modified oligonucleotide.

Embodiment 34. The oligomeric compound of any of embodiments 28-32, wherein the conjugate group is attached to the modified oligonucleotide at the 3′-end of the modified oligonucleotide.

Embodiment 35. The oligomeric compound of any of embodiments 1-26 or 28-34 comprising a terminal group.
Embodiment 36. The oligomeric compound of any of embodiments 1-35 wherein the oligomeric compound is a singled-stranded oligomeric compound.
Embodiment 37. The oligomeric compound of any of embodiments 1-31 or 33-34, wherein the oligomeric compound does not comprise linker-nucleosides.
Embodiment 38. An oligomeric duplex comprising an oligomeric compound of any of embodiments 1-35 and 37.
Embodiment 39. An antisense compound comprising or consisting of an oligomeric compound of any of embodiments 1-37 or an oligomeric duplex of embodiment 38.
Embodiment 40. A modified oligonucleotide consisting of 12 to 50 linked nucleosides and having a nucleobase sequence comprising at least 8, at least 9, at least 10, at least 11, at least 12, at least 13, at least 14, at least 15, at least 16, at least 17, or at least 18 consecutive nucleobases of any of the nucleobase sequences of SEQ ID NOs: 10-321.
Embodiment 41. A pharmaceutical composition comprising an oligomeric compound of any of embodiments 1-37, an oligomeric duplex of embodiment 38, an antisense compound of embodiment 39, or a modified oligonucleotide of embodiment 40 and at least one of a pharmaceutically acceptable carrier or diluent.
Embodiment 42. The pharmaceutical composition of embodiment 41, wherein the modified oligonucleotide is a sodium salt.
Embodiment 43. A method comprising administering to an animal the pharmaceutical composition of any of embodiments 41-42.
Embodiment 44. The method of embodiment 43, wherein the animal is a human.

Embodiment 45. A method of treating a disease associated with STMN2 comprising administering to an individual having or at risk for developing a disease associated with STMN2 a therapeutically effective amount of a pharmaceutical composition of embodiments 41 and 42, and thereby treating the disease associated with STMN2.
Embodiment 46. The method of embodiment 45, wherein the disease associated with STMN2 is a neurodegenerative disease.
Embodiment 47. The method of embodiment 46, wherein the neurodegenerative disease is amyotrophic lateral sclerosis (ALS), frontotemporal dementia (FTD), Alzheimer's disease (AD), and dementia with Lewy bodies (DLB).
Embodiment 48. The method of embodiment 47, wherein at least one symptom of the neurodegenerative disease is ameliorated.

Embodiment 49. The method of embodiment 48, wherein the symptom is ataxia, neuropathy, synaptic dysfunction, deficits in cognition, and decreased longevity.

### I. CERTAIN OLIGONUCLEOTIDES

In certain embodiments, provided herein are oligonucleotides, which consist of linked nucleosides. Oligonucleotides may be unmodified oligonucleotides (RNA or DNA) or may be modified oligonucleotides. Modified oligonucleotides comprise at least one modification relative to unmodified RNA or DNA. That is, modified oligonucleotides comprise at least one modified nucleoside (comprising a modified sugar moiety and/or a modified nucleobase) and/or at least one modified internucleoside linkage.

### A. Certain Modified Nucleosides

Modified nucleosides comprise a modified sugar moiety or a modified nucleobase or both a modified sugar moiety and a modified nucleobase.

### 1. Certain Sugar Moieties

In certain embodiments, modified sugar moieties are non-bicyclic modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified sugar moieties comprising a furanosyl ring with one or more substituent groups none of which bridges two atoms of the furanosyl ring to form a bicyclic structure. Such non bridging substituents may be at any position of the furanosyl, including but not limited to substituents at the 2′, 4′, and/or 5′ positions. In certain embodiments one or more non-bridging substituent of non-bicyclic modified sugar moieties is branched. Examples of 2′-substituent groups suitable for non-bicyclic modified sugar moieties include but are not limited to: 2′-F, 2′-OCH₃ (“OMe” or “O-methyl”), and 2′-O(CH₂)₂OCH₃(“MOE”). In certain embodiments, 2′-substituent groups are selected from among: halo, allyl, amino, azido, SH, CN, OCN, CF₃, OCF₃, O-C₁-C₁₀ alkoxy, O-C₁-C₁₀ substituted alkoxy, O-C₁-C₁₀ alkyl, O-C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. Examples of 4′-substituent groups suitable for non-bicyclic modified sugar moieties include but are not limited to alkoxy (e.g., methoxy), alkyl, and those described in Manoharan et al., WO 2015/106128. Examples of 5′-substituent groups suitable for non-bicyclic modified sugar moieties include but are not limited to: 5′-methyl (R or S), 5′-vinyl, and 5′-methoxy. In certain embodiments, non-bicyclic modified sugar moieties comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836.).

In certain embodiments, a 2′-substituted non-bicyclic modified nucleoside comprises a sugar moiety comprising a non-bridging 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl.

In certain embodiments, a 2′-substituted nucleoside non-bicyclic modified nucleoside comprises a sugar moiety comprising a non-bridging 2′-substituent group selected from: F, OCF₃, OCH₃, OCH₂CH₂OCH₃, O(CH₂)₂SCH, O(CH₂)₂ON(CH₃)₂, O(CH₂)₂O(CH₂)₂(CH₃)₂, and OCH₂C(=O)—N(H)CH₃ (“NMA”).

In certain embodiments, a 2′-substituted non-bicyclic modified nucleoside comprises a sugar moiety comprising a non-bridging 2′-substituent group selected from: F, OCH₃, and OCH₂CH₂OCH₃.

Certain modified sugar moieties comprise a substituent that bridges two atoms of the furanosyl ring to form a second ring, resulting in a bicyclic sugar moiety. In certain such embodiments, the bicyclic sugar moiety comprises a bridge between the 4′ and the 2′ furanose ring atoms. Examples of such 4′ to 2′ bridging sugar substituents include but are not limited to: 4′-CH₂-2′, 4′-(CH₂)₂-2′, 4′-(CH₂)₃-2′, 4′-CH₂—O-2′ (“LNA”), 4′-CH₂-S-2′, 4′-(CH₂)₂-O-2′ (“ENA”), 4′-CH(CH₃)—O-2′ (referred to as “constrained ethyl” or “cEt”), 4′-CH₂—O—CH₂-2′, 4′-CH₂-N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J. Org. Chem., 2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4′-C(RₐR_{b})—O—N(R)-2′, 4′-CH₂—O—N(R)-2′, and 4′-CH₂-N(R)—O-2′, wherein each R, Rₐ, and R_{b} is, independently, H, a protecting group, or C₁-C₁₂ alkyl (see, e.g. Imanishi et al., U.S. Pat. No. 7,427,672).

In certain embodiments, such 4′ to 2′ bridges independently comprise from 1 to 4 linked groups independently selected from: —[C(Rₐ)(R_{b})]ₙ—, —[C(Rₐ)(R_{b})]ₙ—O—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—;
wherein:
x is 0, 1, or 2;
n is 1, 2, 3, or 4;
each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and
each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Freier et al., Nucleic Acids Research, 1997, 25 (22), 4429-4443, Alback et al., J. Org. Chem., 2006, 71, 7731-7740, Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129, 8362-8379; Wengel et a., U.S. Pat. No. 7,053,207; Imanishi et al., U.S. Pat. No. 6,268,490; Imanishi et al. U.S. Pat. No. 6,770,748; Imanishi et al., U.S. RE44,779; Wengel et al., U.S. Pat. No. 6,794,499; Wengel et al., U.S. Pat. No. 6,670,461; Wengel et al., U.S. Pat. No. 7,034,133; Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191; Torsten et al., WO 2004/106356; Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. Patent Publication Nos. Allerson et al., US2008/0039618 and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

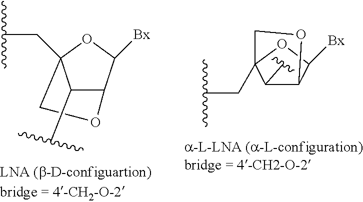

α-L-methyleneoxy (4′-CH₂—O-2′) or α-L-LNA bicyclic nucleosides have been incorporated into oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“MNA”) (see, e.g., Leumann, CJ. Bioorg. & Med. Chem. 2002, 10, 841-854), fluoro HNA:

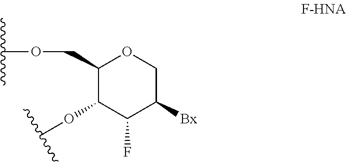

“F-HNA”, see e.g. Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3′-fluoro tetrahydropyran), and nucleosides comprising additional modified THP compounds having the formula:

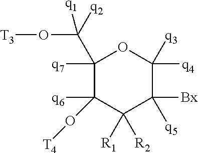

wherein, independently, for each of said modified THP nucleoside:
Bx is a nucleobase moiety;
T₃ and T₄ are each, independently, an internucleoside linking group linking the modified THP nucleoside to the remainder of an oligonucleotide or one of T₃ and T₄ is an internucleoside linking group linking the modified THP nucleoside to the remainder of an oligonucleotide and the other of T₃ and T₄ is H, a hydroxyl protecting group, a linked conjugate group, or a 5′ or 3′-terminal group;
   q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and
each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

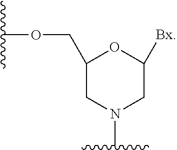

In certain embodiments, morpholinos may be modified, for example by adding or altering various substituent groups from the above morpholino structure. Such sugar surrogates are referred to herein as “modified morpholinos.”

In certain embodiments, sugar surrogates comprise acyclic moieites. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., WO2011/133876.

Many other bicyclic and tricyclic sugar and sugar surrogate ring systems are known in the art that can be used in modified nucleosides).

### 2. Certain Modified Nucleobases

In certain embodiments, modified oligonucleotides comprise one or more nucleoside comprising an unmodified nucleobase. In certain embodiments, modified oligonucleotides comprise one or more nucleoside comprising a modified nucleobase. In certain embodiments, modified oligonucleotides comprise one or more nucleoside that does not comprise a nucleobase, referred to as an abasic nucleoside.

In certain embodiments, modified nucleobases are selected from: 5-substituted pyrimidines, 6-azapyrimidines, alkyl or alkynyl substituted pyrimidines, alkyl substituted purines, and N-2, N-6 and O-6 substituted purines. In certain embodiments, modified nucleobases are selected from: 2-aminopropyladeninc, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (-C=C-CH3) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering. Kroschwitz, J.I., Ed., John Wiley & Sons, 1990, 858-859; Englisch et al., Angewandte Chemie, International Edition, 1991, 30, 613; Sanghvi. Y.S., Chapter 15, Antisense Research and Applications, Crooke, S.T. and Lebleu, B., Eds., CRC Press, 1993, 273-288; and those disclosed in Chapters 6 and 15, Antisense Drug Technology, Crooke S.T., Ed., CRC Press, 2008, 163-166 and 442-443.

Publications that teach the preparation of certain of the above noted modified nucleobases as well as other modified nucleobases include without limitation, Manoharan et al., US2003/0158403; Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Frochler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Frochler et al., U.S. Pat. No. 5,594,121; Switzer et al., U.S. Pat. No. 5,596,091; Cook et al., U.S. Pat. No. 5,614,617; Frochler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Frochler et al., U.S. Pat. No. 5,830,653; Cook et al., U.S. Pat. No. 5,808,027; Cook et al., 6.166.199; and Matteucci et al., U.S. Pat. No. 6,005,096.

### 3. Certain Modified Internucleoside Linkages

In certain embodiments, nucleosides of modified oligonucleotides may be linked together using any internucleoside linkage. The two main classes of internucleoside linking groups are defined by the presence or absence of a phosphorus atom. Representative phosphorus-containing internucleoside linkages include but are not limited to phosphates, which contain a phosphodiester bond (“P═O”) (also referred to as unmodified or naturally occurring linkages), phosphotriesters, methylphosphonates, phosphoramidates, and phosphorothioates (“P═S”), and phosphorodithioates (“HS—P═S”). Representative non-phosphorus containing internucleoside linking groups include but are not limited to methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)—). Modified internucleoside linkages, compared to naturally occurring phosphate linkages, can be used to alter, typically increase, nuclease resistance of the oligonucleotide. In certain embodiments, internucleoside linkages having a chiral atom can be prepared as a racemic mixture, or as separate enantiomers. Methods of preparation of phosphorous-containing and non-phosphorous-containing internucleoside linkages are well known to those skilled in the art.

Representative internucleoside linkages having a chiral center include but are not limited to alkylphosphonates and phosphorothioates. Modified oligonucleotides comprising internucleoside linkages having a chiral center can be prepared as populations of modified oligonucleotides comprising stereorandom internucleoside linkages, or as populations of modified oligonucleotides comprising phosphorothioatc linkages in particular stereochemical configurations. In certain embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate linkage. Nonetheless, as is well understood by those of skill in the art, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkages in a particular, independently selected stereochemical configuration. In certain embodiments, the particular configuration of the particular phosphorothioate linkage is present in at least 65% of the molecules in the population. In certain embodiments, the particular configuration of the particular phosphorothioate linkage is present in at least 70% of the molecules in the population. In certain embodiments, the particular configuration of the particular phosphorothioate linkage is present in at least 80% of the molecules in the population. In certain embodiments, the particular configuration of the particular phosphorothioate linkage is present in at least 90% of the molecules in the population. In certain embodiments, the particular configuration of the particular phosphorothioate linkage is present in at least 99% of the molecules in the population. Such chirally enriched populations of modified oligonucleotides can be generated using synthetic methods known in the art, e.g., methods described in Oka et al., JACS 125, 8307 (2003), Wan et al. Nuc. Acid. Res. 42, 13456 (2014), and WO 2017/015555. In certain embodiments, a population of modified oligonucleotides is enriched for modified oligonucleotides having at least one indicated phosphorothioate in the (Sp) configuration. In certain embodiments, a population of modified oligonucleotides is enriched for modified oligonucleotides having at least one phosphorothioate in the (Rp) configuration. In certain embodiments, modified oligonucleotides comprising (Rp) and/or (Sp) phosphorothioates comprise one or more of the following formulas, respectively, wherein “B” indicates a nucleobase:

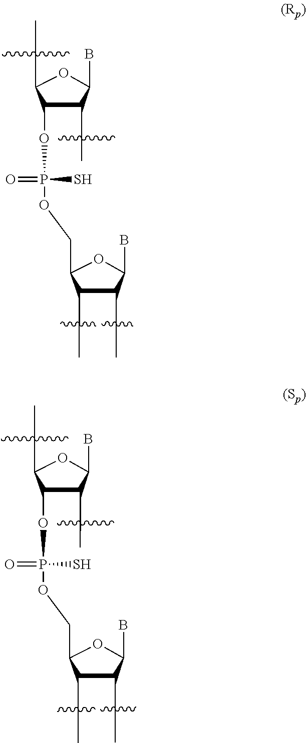

Unless otherwise indicated, chiral internucleoside linkages of modified oligonucleotides described herein can be stereorandom or in a particular stereochemical configuration.

Neutral internucleoside linkages include, without limitation, phosphotriesters, methylphosphonates, MMI (3′-CH₂—N(CH₃)—O-5′), amide-3(3′-CH₂—C(═O)—N(H)-5′), amide-4(3′-CH₂—N(H)—C(═O)-5′), formacetal (3′-O—CH₂—O-5′), methoxypropyl, and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y.S. Sanghvi and P.D. Cook, Eds., ACS Symposium Series 580; Chapters 3 and 4, 40-65). Further neutral internucleoside linkages include nonionic linkages comprising mixed N, O, S and CH₂ component parts.

### B. Certain Motifs

In certain embodiments, modified oligonucleotides comprise one or more modified nucleosides comprising a modified sugar moiety. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

### 1. Certain Sugar Motifs

In certain embodiments, oligonucleotides comprise one or more type of modified sugar and/or unmodified sugar moiety arranged along the oligonucleotide or region thereof in a defined pattern or sugar motif. In certain instances, such sugar motifs include but are not limited to any of the sugar modifications discussed herein.

In certain embodiments, modified oligonucleotides comprise or consist of a region having a gapmer motif, which is defined by two external regions or “wings” and a central or internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap (i.e., the wing/gap junction). In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar motifs of the two wings are the same as one another (symmetric gapmer). In certain embodiments, the sugar motif of the 5′-wing differs from the sugar motif of the 3′-wing (asymmetric gapmer).

In certain embodiments, the wings of a gapmer comprise 1-5 nucleosides. In certain embodiments, each nucleoside of each wing of a gapmer is a modified nucleoside. In certain embodiments, at least one nucleoside of each wing of a gapmer is a modified nucleoside. In certain embodiments, at least two nucleosides of each wing of a gapmer are modified nucleosides. In certain embodiments, at least three nucleosides of each wing of a gapmer are modified nucleosides. In certain embodiments, at least four nucleosides of each wing of a gapmer are modified nucleosides.

In certain embodiments, the gap of a gapmer comprises 7-12 nucleosides. In certain embodiments, each nucleoside of the gap of a gapmer is an unmodified 2′-deoxy nucleoside.

In certain embodiments, the gapmer is a deoxy gapmer. In embodiments, the nucleosides on the gap side of each wing/gap junction are unmodified 2′-deoxy nucleosides and the nucleosides on the wing sides of each wing/gap junction are modified nucleosides. In certain embodiments, each nucleoside of the gap is an unmodified 2′-deoxy nucleoside. In certain embodiments, each nucleoside of each wing of a gapmer is a modified nucleoside.

Herein, the lengths (number of nucleosides) of the three regions of a gapmer may be provided using the notation [#of nucleosides in the 5′-wing]-[#of nucleosides in the gap]-[#of nucleosides in the 3′-wing]. Thus, a 5-10-5 gapmer consists of 5 linked nucleosides in each wing and 10 linked nucleosides in the gap. Where such nomenclature is followed by a specific modification, that modification is the modification in each sugar moiety of each wing and the gap nucleosides comprise unmodified deoxynucleosides sugars. Thus, a 5-10-5 MOE gapmer consists of 5 linked MOE modified nucleosides in the 5′-wing. 10 linked deoxynucleosides in the gap, and 5 linked MOE nucleosides in the 3′-wing.

In certain embodiments, modified oligonucleotides are 5-10-5 MOE gapmers. In certain embodiments, modified oligonucleotides are 3-10-3 BNA gapmers. In certain embodiments, modified oligonucleotides are 3-10-3 cEt gapmers. In certain embodiments, modified oligonucleotides are 3-10-3 LNA gapmers.

In certain embodiments, modified oligonucleotides comprise or consist of a region having a fully modified sugar motif. In such embodiments, each nucleoside of the fully modified region of the modified oligonucleotide comprises a modified sugar moiety. In certain embodiments, modified oligonucleotides comprise or consist of a region having a fully modified sugar motif, wherein each nucleoside within the fully modified region comprises the same modified sugar moiety (uniformly modified sugar motif). In certain embodiments, the uniformly modified sugar motif is 7 to 20 nucleosides in length. In certain embodiments, each nucleoside of the uniformly modified sugar motif is a 2′-substituted nucleoside, a sugar surrogate, or a bicyclic nucleoside. In certain embodiments, each nucleoside of the uniformly modified sugar motif comprises either a 2′-OCH₂CH₂OCH₃ group or a 2′-OCH₃ group. In certain embodiments, modified oligonucleotides having at least one fully modified sugar motif may also have at least 1, at least 2, at least 3, or at least 4 2′-deoxynucleosides.

In certain embodiments, each nucleoside of the entire modified oligonucleotide comprises a modified sugar moiety (fully modified oligonucleotide). In certain embodiments, a fully modified oligonucleotide comprises different 2′-modifications. In certain embodiments, each nucleoside of a fully modified oligonucleotide is a 2-substituted nucleoside, a sugar surrogate, or a bicyclic nucleoside. In certain embodiments, each nucleoside of a fully modified oligonucleotide comprises either a 2′-OCH₂CH₂OCH₃ group and at least one 2′-OCH₃ group.

In certain embodiments, each nucleoside of a fully modified oligonucleotide comprises the same 2′-modification (uniformly modified oligonucleotide). In certain embodiments, each nucleoside of a uniformly modified oligonucleotide is a 2′-substituted nucleoside, a sugar surrogate, or a bicyclic nucleoside. In certain embodiments, each nucleoside of a uniformly modified oligonucleotide comprises either a 2′-OCH₂CH₂OCH₃ group or a 2′-OCH₃ group

In certain embodiments, modified oligonucleotides comprise at least 12, at last 13, at least 14, at least 15, at least 16, at least 17, at least 18, at least 19, or at least 20 nucleosides comprising a modified sugar moiety. In certain embodiments, each nucleoside of a modified oligonucleotide is a 2′-substituted nucleoside, a sugar surrogate, a bicyclic nucleoside, or a 2′-deoxynucleoside. In certain embodiments, each nucleoside of a modified oligonucleotide comprises a 2′-OCH₂CH₂OCH₃ group, a 2′-H(H) deoxyribosyl sugar moiety, or a cEt modified sugar.

### 2. Certain Nucleobase Motifs

In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methyl cytosines. In certain embodiments, all of the cytosine nucleobases are 5-methyl cytosines and all of the other nucleobases of the modified oligonucleotide are unmodified nucleobases.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

In certain embodiments, oligonucleotides having a gapmer motif comprise a nucleoside comprising a modified nucleobase. In certain such embodiments, one nucleoside comprising a modified nucleobase is in the central gap of an oligonucleotide having a gapmer motif. In certain such embodiments, the sugar moiety of said nucleoside is a 2′-deoxyribosyl moiety. In certain embodiments, the modified nucleobase is selected from: a 2-thiopyrimidine and a 5-propynepyrimidine.

### 3. Certain Internucleoside Linkage Motifs

In certain embodiments, oligonucleotides comprise modified and/or unmodified internucleoside linkages arranged along the oligonucleotide or region thereof in a defined pattern or motif. In certain embodiments, each internucleoside linking group is a phosphodiester internucleoside linkage (P═O). In certain embodiments, each internucleoside linking group of a modified oligonucleotide is a phosphorothioate internucleoside linkage (P═S). In certain embodiments, each internucleoside linkage of a modified oligonucleotide is independently selected from a phosphorothioate internucleoside linkage and phosphodiester internucleoside linkage. In certain embodiments, each phosphorothioate internucleoside linkage is independently selected from a stereorandom phosphorothioate a (Sp) phosphorothioate, and a (Rp) phosphorothioate. In certain embodiments, the sugar motif of a modified oligonucleotide is a gapmer and the internucleoside linkages within the gap are all modified. In certain such embodiments, some or all of the internucleoside linkages in the wings are unmodified phosphodiester internucleoside linkages. In certain embodiments, the terminal internucleoside linkages are modified. In certain embodiments, the sugar motif of a modified oligonucleotide is a gapmer, and the internucleoside linkage motif comprises at least one phosphodiester internucleoside linkage in at least one wing, wherein the at least one phosphodiester linkage is not a terminal internucleoside linkage, and the remaining internucleoside linkages are phosphorothioate internucleoside linkages. In certain such embodiments, all of the phosphorothioate linkages are stereorandom. In certain embodiments, all of the phosphorothioate linkages in the wings are (Sp) phosphorothioates, and the gap comprises at least one Sp, Sp, Rp motif. In certain embodiments, populations of modified oligonucleotides are enriched for modified oligonucleotides comprising such internucleoside linkage motifs.

### C. Certain Lengths

It is possible to increase or decrease the length of an oligonucleotide without eliminating activity. For example, in Woolf et al. (Proc. Natl. Acad. Sci. USA 89:7305-7309, 1992), a series of oligonucleotides 13-25 nucleobases in length were tested for their ability to induce cleavage of a target RNA in an oocyte injection model. Oligonucleotides 25 nucleobases in length with 8 or 11 mismatch bases near the ends of the oligonucleotides were able to direct specific cleavage of the target RNA, albeit to a lesser extent than the oligonucleotides that contained no mismatches. Similarly, target specific cleavage was achieved using 13 nucleobase oligonucleotides, including those with 1 or 3 mismatches.

In certain embodiments, oligonucleotides (including modified oligonucleotides) can have any of a variety of ranges of lengths. In certain embodiments, oligonucleotides consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides

### D. Certain Modified Oligonucleotides

In certain embodiments, the above modifications (sugar, nucleobase, internucleoside linkage) are incorporated into a modified oligonucleotide. In certain embodiments, modified oligonucleotides are characterized by their modification motifs and overall lengths. In certain embodiments, such parameters are each independent of one another. Thus, unless otherwise indicated, each internucleoside linkage of an oligonucleotide having a gapmer sugar motif may be modified or unmodified and may or may not follow the gapmer modification pattern of the sugar modifications. For example, the internucleoside linkages within the wing regions of a sugar gapmer may be the same or different from one another and may be the same or different from the internucleoside linkages of the gap region of the sugar motif. Likewise, such sugar gapmer oligonucleotides may comprise one or more modified nucleobase independent of the gapmer pattern of the sugar modifications. Unless otherwise indicated, all modifications are independent of nucleobase sequence.

### E. Certain Populations of Modified Oligonucleotides

Populations of modified oligonucleotides in which all of the modified oligonucleotides of the population have the same molecular formula can be stereorandom populations or chirally enriched populations. All of the chiral centers of all of the modified oligonucleotides are stereorandom in a stereorandom population. In a chirally enriched population, at least one particular chiral center is not stereorandom in the modified oligonucleotides of the population. In certain embodiments, the modified oligonucleotides of a chirally enriched population are enriched for β-D ribosyl sugar moieties, and all of the phosphorothioate internucleoside linkages are stereorandom. In certain embodiments, the modified oligonucleotides of a chirally enriched population are enriched for both β-D ribosyl sugar moieties and at least one, particular phosphorothioate internucleoside linkage in a particular stereochemical configuration.

### F. Nucleobase Sequence

In certain embodiments, oligonucleotides (unmodified or modified oligonucleotides) are further described by their nucleobase sequence. In certain embodiments oligonucleotides have a nucleobase sequence that is complementary to a second oligonucleotide or an identified reference nucleic acid, such as a target nucleic acid. In certain such embodiments, a region of an oligonucleotide has a nucleobase sequence that is complementary to a second oligonucleotide or an identified reference nucleic acid, such as a target nucleic acid. In certain embodiments, the nucleobase sequence of a region or entire length of an oligonucleotide is at least 50%, at least 60%, at least 70%, at least 80%, at least 85%, at least 90%, at least 95%, or 100% complementary to the second oligonucleotide or nucleic acid, such as a target nucleic acid.

### II. CERTAIN OLIGOMERIC COMPOUNDS

In certain embodiments, provided herein are oligomeric compounds, which consist of an oligonucleotide (modified or unmodified) and optionally one or more conjugate groups and/or terminal groups. Conjugate groups consist of one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to cither or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3″-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphate moietics, protecting groups, modified or unmodified nucleosides, and two or more nucleosides that are independently modified or unmodified.

### A. Certain Conjugate Groups

In certain embodiments, oligonucleotides are covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge and clearance. In certain embodiments, conjugate groups impart a new property on the attached oligonucleotide, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060) , a thioether, e.g., hexyl-S-tritylthiol (Manoharan et al., Ann. N.Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al,, FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GalNAc cluster (e.g., WO2014/179620).

### 1. Conjugate Moieties

Conjugate moieties include, without limitation, intercalators, reporter molecules, polyamines, polyamides, peptides, carbohydrates, vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

### 2. Conjugate Linkers

Conjugate moieties are attached to oligonucleotides through conjugate linkers. In certain oligomeric compounds, the conjugate linker is a single chemical bond (i.e., the conjugate moiety is attached directly to an oligonucleotide through a single bond). In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to parent compounds, such as the oligonucleotides provided herein. In general, a bifunctional linking moiety comprises at least two functional groups. One of the functional groups is selected to bind to a particular site on a parent compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-malcimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxy, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, conjugate linkers comprise 2-5 linker-nucleosides. In certain embodiments, conjugate linkers comprise exactly 3 linker-nucleosides. In certain embodiments, conjugate linkers comprise the TCA motif. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methyl cytosine, 4-N-benzoyl-5-methyl cytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such an oligomeric compound is more than 30. Alternatively, an oligomeric compound may comprise a modified oligonucleotide consisting of 8-30 nucleosides and no conjugate group. The total number of contiguous linked nucleosides in such an oligomeric compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances oligomeric compounds comprising a particular conjugate moiety are better taken up by a particular cell type, but once the oligomeric compound has been taken up, it is desirable that the conjugate group be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides. In certain such embodiments, the one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxy nucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate linkage. In certain such embodiments, the cleavable moiety is 2′-deoxyadenosine.

### B. Certain Terminal Groups

In certain embodiments, oligomeric compounds comprise one or more terminal groups. In certain such embodiments, oligomeric compounds comprise a stabilized 5′-phophate. Stabilized 5-phosphates include, but are not limited to 5′-phosphanates, including, but not limited to 5′-vinylphosphonates. In certain embodiments, terminal groups comprise one or more abasic nucleosides and/or inverted nucleosides. In certain embodiments, terminal groups comprise one or more 2′-linked nucleosides. In certain such embodiments, the 2′-linked nucleoside is an abasic nucleoside.

### III. OLIGOMERIC DUPLEXES

In certain embodiments, oligomeric compounds described herein comprise an oligonucleotide, having a nucleobase sequence complementary to that of a target nucleic acid. In certain embodiments, an oligomeric compound is paired with a second oligomeric compound to form an oligomeric duplex. Such oligomeric duplexes comprise a first oligomeric compound having a region complementary to a target nucleic acid and a second oligomeric compound having a region complementary to the first oligomeric compound. In certain embodiments, the first oligomeric compound of an oligomeric duplex comprises or consists of (1) a modified or unmodified oligonucleotide and optionally a conjugate group and (2) a second modified or unmodified oligonucleotide and optionally a conjugate group. Either or both oligomeric compounds of an oligomeric duplex may comprise a conjugate group. The oligonucleotides of each oligomeric compound of an oligomeric duplex may include non-complementary overhanging nucleosides.

### IV. ANTISENSE ACTIVITY

In certain embodiments, oligomeric compounds and oligomeric duplexes are capable of hybridizing to a target nucleic acid, resulting in at least one antisense activity; such oligomeric compounds and oligomeric duplexes are antisense compounds. In certain embodiments, antisense compounds have antisense activity when they increase the amount or activity of a target nucleic acid by 25% or more in the standard cell assay. In certain embodiments, antisense compounds selectively affect one or more target nucleic acid. Such antisense compounds comprise a nucleobase sequence that hybridizes to one or more target nucleic acid. resulting in one or more desired antisense activity and does not hybridize to one or more non-target nucleic acid or does not hybridize to one or more non-target nucleic acid in such a way that results in significant undesired antisense activity.

In certain antisense activities, hybridization of an antisense compound to a target nucleic acid results in recruitment of a protein that cleaves the target nucleic acid. For example, certain antisense compounds result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, described herein are antisense compounds that are sufficiently “DNA-like” to elicit RNase H activity. In certain embodiments, one or more non-DNA-like nucleoside in the gap of a gapmer is tolerated.

In certain antisense activities, an antisense compound or a portion of an antisense compound is loaded into an RNA-induced silencing complex (RISC), ultimately resulting in cleavage of the target nucleic acid. For example, certain antisense compounds result in cleavage of the target nucleic acid by Argonaute. Antisense compounds that are loaded into RISC are RNAi compounds. RNAi compounds may be double-stranded (siRNA) or single-stranded (ssRNA).

In certain embodiments, hybridization of an antisense compound to a target nucleic acid does not result in recruitment of a protein that cleaves that target nucleic acid. In certain embodiments, hybridization of the antisense compound to the target nucleic acid results in alteration of splicing of the target nucleic acid. In certain embodiments, hybridization of an antisense compound to a target nucleic acid results in inhibition of a binding interaction between the target nucleic acid and a protein or other nucleic acid. In certain embodiments, hybridization of an antisense compound to a target nucleic acid results in alteration of translation of the target nucleic acid. In certain embodiments, hybridization of an antisense compound to a target nucleic acid results in an increase in the amount or activity of a target nucleic acid.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein, and/or a phenotypic change in a cell or animal.

### V. CERTAIN TARGET NUCLEIC ACIDS

In certain embodiments, oligomeric compounds comprise or consist of an oligonucleotide comprising a region that is complementary to a target nucleic acid. In certain embodiments, the target nucleic acid is an endogenous RNA molecule. In certain embodiments, the target nucleic acid encodes a protein. In certain such embodiments, the target nucleic acid is selected from: a mature RNA and a pre-mRNA, including intronic, exonic and untranslated regions. In certain embodiments, the target RNA is a mature RNA. In certain embodiments, the target nucleic acid is a pre-mRNA. In certain such embodiments, the target region is entirely within an intron. In certain embodiments, the target region spans an intron/exon junction. In certain embodiments, the target region is at least 50% within an intron. In certain embodiments, the target nucleic acid is the RNA transcriptional product of a retrogene. In certain embodiments, the target nucleic acid is a non-coding RNA. In certain such embodiments, the target non-coding RNA is selected from: a long non-coding RNA, a short non-coding RNA, an intronic RNA molecule.

### A. Complementarity/Mismatches to the Target Nucleic Acid

It is possible to introduce mismatch bases without eliminating activity. For example, Gautschi et al (J. Natl. Cancer Inst. 93:463-471, March 2001) demonstrated the ability of an oligonucleotide having 100% complementarity to the bel-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo. Maher and Dolnick (Nuc. Acid. Res. 16:3341-3358, 1988) tested a series of tandem 14 nucleobase oligonucleotides, and a 28 and 42 nucleobase oligonucleotides comprised of the sequence of two or three of the tandem oligonucleotides, respectively, for their ability to arrest translation of human DHFR in a rabbit reticulocyte assay. Each of the three 14 nucleobase oligonucleotides alone was able to inhibit translation, albeit at a more modest level than the 28 or 42 nucleobase oligonucleotides.

In certain embodiments, oligonucleotides that are complementary to the target nucleic acid over the entire length of the oligonucleotide. In certain embodiments, oligonucleotides are 99%, 95%, 90%, 85%, or 80% complementary to the target nucleic acid. In certain embodiments, oligonucleotides are at least 80% complementary to the target nucleic acid over the entire length of the oligonucleotide and comprise a region that is 100% or fully complementary to a target nucleic acid. In certain embodiments, the region of full complementarity is from 6 to 20, 10 to 18, or 18 to 20 nucleobases in length.

In certain embodiments, oligonucleotides comprise one or more mismatched nucleobases relative to the target nucleic acid. In certain embodiments, antisense activity against the target is reduced by such mismatch, but activity against a non-target is reduced by a greater amount. Thus, in certain embodiments selectivity of the oligonucleotide is improved. In certain embodiments, the mismatch is specifically positioned within an oligonucleotide having a gapmer motif. In certain embodiments, the mismatch is at position 1, 2, 3, 4, 5, 6, 7, or 8 from the 5′-end of the gap region. In certain embodiments, the mismatch is at position 9, 8, 7, 6, 5, , 3, 2, 1 from the 3′-end of the gap region. In certain embodiments, the mismatch is at position 1, 2, 3, or 4 from the 5′-end of the wing region. In certain embodiments, the mismatch is at position 4, 3, 2, or 1 from the 3′-end of the wing region.

### B. STMN2

In certain embodiments, oligomeric compounds comprise or consist of an oligonucleotide comprising a region that is complementary to a target nucleic acid, wherein the target nucleic acid is STMN2. In certain embodiments, STMN2 nucleic acid has the sequence set forth in SEQ ID NO: 1 (complement of GENBANK Accession No. NC_000008.11 truncated from nucleobase 79608001 to 79669000) and SEQ ID NO: 2.

In certain embodiments, contacting a cell with an oligomeric compound complementary to SEQ ID NO: 1 or SEQ ID NO: 2 increases the amount of STMN2 RNA, and in certain embodiments increases the amount of stathmin-2 protein. In certain embodiments, contacting a cell in an animal with an oligomeric compound complementary to SEQ ID NO: 1 or SEQ ID NO: 2 ameliorates one or more symptoms of a neurodegenerative disease. Such symptoms include ataxia, neuropathy, synaptic dysfunction, deficits in cognition, and decreased longevity. In certain embodiments, contacting a cell in an animal with an oligonucleotide complementary to SEQ ID NO: 1 or SEQ ID NO: 2 results in improved motor function, reduced neuropathy, improved synaptic function, improved cognition, and survival.

### VI. CERTAIN HOTSPOT REGIONS

### 1. Nucleobases 8819-8841 of SEQ ID NO: 1 (Also 100-122 of SEQ ID NO: 2)

In certain embodiments, nucleobases 8819-8841 of SEQ ID NO: 1 comprise a hotspot region. In certain embodiments, modified oligonucleotides are complementary to nucleobases 8819-8841 of SEQ ID NO: 1. In certain embodiments, such modified oligonucleotides are 18 nucleobases in length. In certain embodiments, such modified oligonucleotides are uniformly MOE modified oligonucleotides. In certain embodiments, the nucleosides of the modified oligonucleotides are linked by phosphorothioate internucleoside linkages.

The nucleobase sequences of SEQ ID Nos: 32, 33, 110, 188, 265, 266 are complementary to nucleobases 8819-8841 of SEQ ID NO: 1.

In certain embodiments. modified oligonucleotides complementary to nucleobases 8819-8841 of SEQ ID NO: 1 achieve at least 151% expression of STMN2 RNA in vitro in the standard cell assay.

### 2. Nucleobases 8827-8851 of SEQ ID NO: 1 (Also 108-132 of SEQ ID NO: 2)

In certain embodiments, nucleobases 8827-8851 of SEQ ID NO: 1 comprise a hotspot region. In certain embodiments, modified oligonucleotides are complementary to nucleobases 8827-8851 of SEQ ID NO: 1. In certain embodiments, such modified oligonucleotides are 18 nucleobases in length. In certain embodiments, such modified oligonucleotides are uniformly MOE modified oligonucleotides. In certain embodiments, the nucleosides of the modified oligonucleotides are linked by phosphorothioate internucleoside linkages.

The nucleobase sequences of SEQ ID Nos: 34, 35, 112, 113, 190, 191, 267, and 268 are complementary to nucleobases 8819-8841 of SEQ ID NO: 1.

In certain embodiments, modified oligonucleotides complementary to nucleobases 8827-8851 of SEQ ID NO: 1 achieve at least 146% expression of STMN2 RNA in vitro in the standard cell assay.

### 3. Nucleobases 8836-8880 of SEQ ID NO: 1 (Also 117-161 of SEQ ID NO: 2)

In certain embodiments, nucleobases 8836-8880 of SEQ ID NO: 1 comprise a hotspot region. In certain embodiments, modified oligonucleotides are complementary to nucleobases 8836-8880 of SEQ ID NO: 1. In certain embodiments, such modified oligonucleotides are 18 nucleobases in length. In certain embodiments, such modified oligonucleotides are uniformly MOE modified oligonucleotides. In certain embodiments, the nucleosides of the modified oligonucleotides are linked by phosphorothioate internucleoside linkages.

The nucleobase sequences of SEQ ID Nos: 36-42, 114-120, 192-198, and 270-276 are complementary to nucleobases 8836-8880 of SEQ ID NO: 1.

In certain embodiments, modified oligonucleotides complementary to nucleobases 8836-8880 of SEQ ID NO: 1 achieve at least 136% expression of STMN2 RNA in vitro in the standard cell assay.

### 4. Nucleobases 8913-8948 of SEQ ID NO: 1 (Also 194-229 of SEQ ID NO: 2)

In certain embodiments, nucleobases 8913-8948 of SEQ ID NO: 1 comprise a hotspot region. In certain embodiments, modified oligonucleotides are complementary to nucleobases 8913-8948 of SEQ ID NO: 1. In certain embodiments, such modified oligonucleotides are 18 nucleobases in length. In certain embodiments, such modified oligonucleotides are uniformly MOE modified oligonucleotides. In certain embodiments, the nucleosides of the modified oligonucleotides are linked by phosphorothioate internucleoside linkages.

The nucleobase sequences of SEQ ID Nos: 56-59, 133-137, 211-215, and 289-293 are complementary to nucleobases 8913-8948 of SEQ ID NO: 1.

In certain embodiments, modified oligonucleotides complementary to nucleobases 8913-8948 of SEQ ID NO: 1 achieve at least 150% expression of STMN2 RNA in vitro in the standard cell assay.

### C. Certain Target Nucleic Acids in Certain Tissues

In certain embodiments, oligomeric compounds comprise or consist of an oligonucleotide comprising a region that is complementary to a target nucleic acid, wherein the target nucleic acid is expressed in a pharmacologically relevant tissue. In certain embodiments, the pharmacologically relevant tissues are the cells and tissues that comprise the central nervous system (CNS), including spinal cord, cortex, cerebellum, and pons.

### VII. CERTAIN PHARMACEUTICAL COMPOSITIONS

In certain embodiments, described herein are pharmaceutical compositions comprising one or more oligomeric compounds. In certain embodiments, the one or more oligomeric compounds each consists of a modified oligonucleotide. In certain embodiments, the pharmaceutical composition comprises a pharmaceutically acceptable diluent or carrier. In certain embodiments, a pharmaceutical composition comprises or consists of a sterile saline solution and one or more oligomeric compound. In certain embodiments, the sterile saline is pharmaceutical grade saline. In certain embodiments, a pharmaceutical composition comprises or consists of one or more oligomeric compound and sterile water. In certain embodiments, the sterile water is pharmaceutical grade water. In certain embodiments, a pharmaceutical composition comprises or consists of one or more oligomeric compound and phosphate-buffered saline (PBS). In certain embodiments, the sterile PBS is pharmaceutical grade PBS. In certain embodiments, a pharmaceutical composition comprises or consists of one or more oligomeric compound and artificial cerebrospinal fluid. In certain embodiments, the artificial cerebrospinal fluid is pharmaceutical grade.

In certain embodiments, a pharmaceutical composition comprises a modified oligonucleotide and artificial cerebrospinal fluid. In certain embodiments, a pharmaceutical composition consists of a modified oligonucleotide and artificial cerebrospinal fluid. In certain embodiments, a pharmaceutical composition consists essentially of a modified oligonucleotide and artificial cerebrospinal fluid. In certain embodiments, the artificial cerebrospinal fluid is pharmaceutical grade.

In certain embodiments pharmaceutical compositions comprise one or more oligomeric compound and one or more excipients. In certain embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, ligomeric compounds may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments, pharmaceutical compositions comprising an oligomeric compound encompass any pharmaceutically acceptable salts of the oligomeric compound, esters of the oligomeric compound, or salts of such esters. In certain embodiments, pharmaceutical compositions comprising oligomeric compounds comprising one or more oligonucleotide, upon administration to an animal, including a human, are capable of providing (directly or indirectly) the biologically active metabolite or residue thereof. Accordingly, for example, the disclosure is also drawn to pharmaceutically acceptable salts of oligomeric compounds, prodrugs, pharmaceutically acceptable salts of such prodrugs, and other bioequivalents. Suitable pharmaceutically acceptable salts include, but are not limited to, sodium and potassium salts. In certain embodiments, prodrugs comprise one or more conjugate group attached to an oligonucleotide, wherein the conjugate group is cleaved by endogenous nucleases within the body.

Lipid moieties have been used in nucleic acid therapies in a variety of methods. In certain such methods, the nucleic acid, such as an oligomeric compound, is introduced into preformed liposomes or lipoplexes made of mixtures of cationic lipids and neutral lipids. In certain methods, DNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In certain embodiments, pharmaceutical compositions comprise a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, pharmaceutical compositions comprise one or more tissue-specific delivery molecules designed to deliver the one or more pharmaceutical agents of the present invention to specific tissues or cell types. For example, in certain embodiments, pharmaceutical compositions include liposomes coated with a tissue-specific antibody.

In certain embodiments, pharmaceutical compositions comprise a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol. 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™, the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions are prepared for oral administration. In certain embodiments, pharmaceutical compositions are prepared for buccal administration. In certain embodiments, a pharmaceutical composition is prepared for administration by injection (e.g., intravenous, subcutaneous, intramuscular, intrathecal, intracerebroventricular, etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiment, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes.

### Nonlimiting Disclosure and Incorporation by Reference

Each of the literature and patent publications listed herein is incorporated by reference in its entirety.

While certain compounds, compositions and methods described herein have been described with specificity in accordance with certain embodiments, the following examples serve only to illustrate the compounds described herein and are not intended to limit the same. Each of the references, GenBank accession numbers, and the like recited in the present application is incorporated herein by reference in its entirety.

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH in place of one 2′-H of DNA) or as an RNA having a modified base (thymine (methylated uracil) in place of a uracil of RNA). Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified nucleobases, such as “AT^{m}CGAUCG.” wherein ^{m}C indicates a cytosine base comprising a methyl group at the 5-position.

Certain compounds described herein (e.g., modified oligonucleotides) have one or more asymmetric center and thus give rise to enantiomers, diastereomers, and other stereoisomeric configurations that may be defined, in terms of absolute stereochemistry, as (R) or (S), as α or β such as for sugar anomers, or as (D) or (L), such as for amino acids, etc. Compounds provided herein that are drawn or described as having certain stereoisomeric configurations include only the indicated compounds. Compounds provided herein that are drawn or described with undefined stereochemistry included all such possible isomers, including their stereorandom and optically pure forms, unless specified otherwise. Likewise, all tautomeric forms of the compounds herein are also included unless otherwise indicated. Unless otherwise indicated, compounds described herein are intended to include corresponding salt forms.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ¹²C, ¹⁵N in place of ¹⁴N, ¹⁷O or ¹⁸O in place of ¹⁶O, and ³³S, ³⁴, ³⁵S, or ³⁶S in place of ³²S. In certain embodiments, non-radioactive isotopic substitutions may impart new properties on the oligomeric compound that are beneficial for use as a therapeutic or research tool. In certain embodiments, radioactive isotopic substitutions may make the compound suitable for research or diagnostic purposes such as imaging.

### EXAMPLES

The following examples illustrate certain embodiments of the present disclosure and are not limiting. Moreover, where specific embodiments are provided, the inventors have contemplated generic application of those specific embodiments. For example, disclosure of an oligonucleotide having a particular motif provides reasonable support for additional oligonucleotides having the same or similar motif. And, for example, where a particular high-affinity modification appears at a particular position, other high-affinity modifications at the same position are considered suitable, unless otherwise indicated.

### Example 1: Effect of Uniformly MOE Modified Oligonucleotides With Phosphorothioate Internucleoside Linkages on Human STMN2 In Vitro, Single Dose

Modified oligonucleotides complementary to a human STMN2 nucleic acid were designed and tested for their effect on STMN2 RNA cultured CRISPR-edited SH-SY5Y cells. SH-SY5Y cells were genetically engineered to express a familial ALS-causing mutation (asparagine substituted to serine at amino acid 352-TDP-43N352S) from both endogenous TDP-43 alleles using a CRISPR-Cas9 site selective nuclease.

Cultured CRISPR-edited SH-SY5Y cells at a density of 20,000 cells per well were transfected using electroporation with 7,000 nM concentration of modified oligonucleotide or no modified oligonucleotide for untreated controls. After approximately 24 hours, RNA was isolated from the cells and STMN2 RNA levels were measured by quantitative real-time PCR. Human primer probe set RTS40280 (forward sequence CCACGAACTTTAGCTTCTCCA, designated herein as SEQ ID NO: 3: reverse sequence GCCAATTGTTTCAGCACCTG, designated herein as SEQ ID NO: 4: probe sequence ACTTTCTTCTTTCCTCTGCAGCCTCC, designated herein as SEQ ID NO: 5) was used to measure mRNA levels. STMN2 mRNA levels were adjusted according to total RNA content, as measured by RiboGreen®. Results are presented in the tables below as percent control of the amount of STMN2 RNA, relative to untreated control cells.

The modified oligonucleotides in the tables below are uniformly modified oligonucleotides. The oligonucleotides are 18 nucleobases in length and each nucleoside has a 2′-MOE group. Each internucleoside linkage is a phosphorothioate internucleoside linkage and each cytosine residue is a 5-methyl cytosine. “Start Site” indicates the 5′-most nucleoside to which the gapmer is complementary in the human nucleic acid sequence. “Stop Site” indicates the 3′-most nucleoside to which the modified oligonucleotide is complementary in the human nucleic acid sequence.

Each modified oligonucleotide listed in the tables below is complementary to human STMN2 nucleic acid sequences SEQ ID NO: 1 and SEQ ID NO: 2, as indicated. As shown below, modified oligonucleotides complementary to human STMN2 increased the amount of human STMN2 RNA.

TABLE 1
Percent control of human STMN2 RNA with uniformly MOE modified
oligonucleotides with phosphorothioate internucleoside linkages

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | STMN2 | SEQ |
| Compound | Start | Stop | Start | Stop | | (% | ID |
| Number | Site | Site | Site | Site | Sequence (5′ to 3′) | control) | NO |
| 1186531 | 8732 | 8749 | 13 | 30 | AATAAAATCCCCAGGTAT | 97 | 10 |
| 1186535 | 8736 | 8753 | 17 | 34 | GAGTAATAAAATCCCCAG | 103 | 11 |
| 1186539 | 8740 | 8757 | 21 | 38 | CCCAGAGTAATAAAATCC | 42 | 12 |
| 1186543 | 8744 | 8761 | 25 | 42 | AATTCCCAGAGTAATAAA | 86 | 13 |
| 1186547 | 8748 | 8765 | 29 | 46 | ACATAATTCCCAGAGTAA | 81 | 14 |
| 1186551 | 8752 | 8769 | 33 | 50 | GAACACATAATTCCCAGA | 70 | 15 |
| 1186555 | 8756 | 8773 | 37 | 54 | GGCAGAACACATAATTCC | 103 | 16 |
| 1186559 | 8760 | 8777 | 41 | 58 | ATGGGGCAGAACACATAA | 91 | 17 |
| 1186563 | 8764 | 8781 | 45 | 62 | AGTGATGGGGCAGAACAC | 88 | 18 |
| 1186567 | 8768 | 8785 | 49 | 66 | AGAGAGTGATGGGGCAGA | 113 | 19 |
| 1186571 | 8772 | 8789 | 53 | 70 | TAAGAGAGAGTGATGGGG | 115 | 20 |
| 1186575 | 8776 | 8793 | 57 | 74 | CAATTAAGAGAGAGTGAT | 112 | 21 |
| 1186579 | 8780 | 8797 | 61 | 78 | AATCCAATTAAGAGAGAG | 165 | 22 |
| 1186583 | 8784 | 8801 | 65 | 82 | TAAAAATCCAATTAAGAG | 166 | 23 |
| 1186587 | 8788 | 8805 | 69 | 86 | ATTTTAAAAATCCAATTA | 116 | 24 |
| 1186591 | 8792 | 8809 | 73 | 90 | TATAATTTTAAAAATCCA | 138 | 25 |
| 1186595 | 8796 | 8813 | 77 | 94 | TGAATATAATTTTAAAAA | 109 | 26 |
| 1186599 | 8800 | 8817 | 81 | 98 | AATATGAATATAATTTTA | 103 | 27 |
| 1186603 | 8804 | 8821 | 85 | 102 | CTGCAATATGAATATAAT | 156 | 28 |
| 1186607 | 8808 | 8825 | 89 | 106 | AGTCCTGCAATATGAATA | 192 | 29 |
| 1186611 | 8812 | 8829 | 93 | 110 | GCCGAGTCCTGCAATATG | 149 | 30 |
| 1186615 | 8816 | 8833 | 97 | 114 | TTCTGCCGAGTCCTGCAA | 127 | 31 |
| 1186619 | 8820 | 8837 | 101 | 118 | GGTCTTCTGCCGAGTCCT | 173 | 32 |
| 1186623 | 8824 | 8841 | 105 | 122 | CGAAGGTCTTCTGCCGAG | 175 | 33 |
| 1186627 | 8828 | 8845 | 109 | 126 | CTCTCGAAGGTCTTCTGC | 204 | 34 |
| 1186631 | 8832 | 8849 | 113 | 130 | CTTTCTCTCGAAGGTCTT | 190 | 35 |
| 1186635 | 8836 | 8853 | 117 | 134 | CTACCTTTCTCTCGAAGG | 160 | 36 |
| 1186639 | 8840 | 8857 | 121 | 138 | TTTTCTACCTTTCTCTCG | 173 | 37 |
| 1186643 | 8844 | 8861 | 125 | 142 | CTTATTTTCTACCTTTCT | 172 | 38 |
| 1186647 | 8848 | 8865 | 129 | 146 | AATTCTTATTTTCTACCT | 153 | 39 |
| 1186651 | 8852 | 8869 | 133 | 150 | GCCAAATTCTTATTTTCT | 211 | 40 |
| 1186655 | 8856 | 8873 | 137 | 154 | GAGAGCCAAATTCTTATT | 222 | 41 |
| 1186659 | 8860 | 8877 | 141 | 158 | CACAGAGAGCCAAATTCT | 193 | 42 |
| 1186663 | 8864 | 8881 | 145 | 162 | CTCACACAGAGAGCCAAA | 131 | 43 |
| 1186667 | 8868 | 8885 | 149 | 166 | CATGCTCACACAGAGAGC | 132 | 44 |
| 1186671 | 8872 | 8889 | 153 | 170 | CACACATGCTCACACAGA | 205 | 45 |
| 1186675 | 8876 | 8893 | 157 | 174 | CACGCACACATGCTCACA | 156 | 46 |
| 1186679 | 8880 | 8897 | 161 | 178 | CACACACGCACACATGCT | 157 | 47 |
| 1186683 | 8884 | 8901 | 165 | 182 | CTCGCACACACGCACACA | 131 | 48 |
| 1186687 | 8888 | 8905 | 169 | 186 | CTCTCTCGCACACACGCA | 152 | 49 |
| 1186691 | 8892 | 8909 | 173 | 190 | CTCTCTCTCTCGCACACA | 182 | 50 |
| 1186695 | 8896 | 8913 | 177 | 194 | CTGTCTCTCTCTCTCGCA | 162 | 51 |
| 1186699 | 8900 | 8917 | 181 | 198 | CTGTCTGTCTCTCTCTCT | 163 | 52 |
| 1186703 | 8904 | 8921 | 185 | 202 | CAGGCTGTCTGTCTCTCT | 117 | 53 |
| 1186707 | 8908 | 8925 | 189 | 206 | TAGGCAGGCTGTCTGTCT | 96 | 54 |
| 1186711 | 8912 | 8929 | 193 | 210 | TTCTTAGGCAGGCTGTCT | 136 | 55 |
| 1186715 | 8916 | 8933 | 197 | 214 | TTTCTTCTTAGGCAGGCT | 209 | 56 |
| 1186719 | 8920 | 8937 | 201 | 218 | TTCATTTCTTCTTAGGCA | 196 | 57 |
| 1186723 | 8924 | 8941 | 205 | 222 | CACATTCATTTCTTCTTA | 199 | 58 |
| 1186727 | 8928 | 8945 | 209 | 226 | CATTCACATTCATTTCTT | 218 | 59 |
| 1186731 | 8932 | 8949 | 213 | 230 | GCCGCATTCACATTCATT | 133 | 60 |
| 1186735 | 8936 | 8953 | 217 | 234 | ACAAGCCGCATTCACATT | 182 | 61 |
| 1186739 | 8940 | 8957 | 221 | 238 | TGCCACAAGCCGCATTCA | 164 | 62 |
| 1186743 | 8944 | 8961 | 225 | 242 | ACTGTGCCACAAGCCGCA | 139 | 63 |
| 1186747 | 8948 | 8965 | 229 | 246 | GTCAACTGTGCCACAAGC | 166 | 64 |
| 1186751 | 8952 | 8969 | 233 | 250 | CCTTGTCAACTGTGCCAC | 179 | 65 |
| 1186755 | 8956 | 8973 | 237 | 254 | TCATCCTTGTCAACTGTG | 155 | 66 |
| 1186759 | 8960 | 8977 | 241 | 258 | TTTATCATCCTTGTCAAC | 123 | 67 |
| 1186763 | 8964 | 8981 | 245 | 262 | TTGATTTATCATCCTTGT | 118 | 68 |
| 1186767 | 8968 | 8985 | 249 | 266 | ATTATTGATTTATCATCC | 115 | 69 |
| 1186771 | 8972 | 8989 | 253 | 270 | TTGCATTATTGATTTATC | 67 | 70 |
| 1186775 | 8976 | 8993 | 257 | 274 | AAGCTTGCATTATTGATT | 55 | 71 |
| 1186779 | 8980 | 8997 | 261 | 278 | TAGTAAGCTTGCATTATT | 78 | 72 |
| 1186783 | 8984 | 9001 | 265 | 282 | ATGATAGTAAGCTTGCAT | 80 | 73 |
| 1186787 | 8988 | 9005 | 269 | 286 | ATAAATGATAGTAAGCTT | 74 | 74 |
| 1186791 | 8992 | 9009 | 273 | 290 | ATTCATAAATGATAGTAA | 47 | 75 |
| 1186795 | 8996 | 9013 | 277 | 294 | TGCTATTCATAAATGATA | 54 | 76 |
| 1186799 | 9000 | 9017 | 281 | 298 | GTATTGCTATTCATAAAT | 64 | 77 |
| 1186803 | 9004 | 9021 | 285 | 302 | TTCAGTATTGCTATTCAT | 69 | 78 |
| 1186807 | 9008 | 9025 | 289 | 306 | TTTCTTCAGTATTGCTAT | 99 | 79 |
| 1186811 | 9012 | 9029 | 293 | 310 | TTAATTTCTTCAGTATTG | 127 | 80 |
| 1186815 | 9016 | 9033 | 297 | 314 | TGTTTTAATTTCTTCAGT | 124 | 81 |
| 1186819 | 9020 | 9037 | 301 | 318 | CTTTTGTTTTAATTTCTT | 83 | 82 |
| 1186823 | 9024 | 9041 | 305 | 322 | CAATCTTTTGTTTTAATT | 111 | 83 |
| 1186827 | 9028 | 9045 | 309 | 326 | ACAGCAATCTTTTGTTTT | 116 | 84 |
| 1186831 | 9032 | 9049 | 313 | 330 | TGAGACAGCAATCTTTTG | 147 | 85 |
| 1186835 | 9036 | 9053 | 317 | 334 | ATATTGAGACAGCAATCT | 103 | 86 |
| 1186839 | 9040 | 9057 | 321 | 338 | AGATATATTGAGACAGCA | 125 | 87 |

TABLE 2
Percent control of human STMN2 RNA with uniformly MOE modified
oligonucleotides with phosphorothioate internucleoside linkages

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | STMN2 | SEQ |
| Compound | Start | Stop | Start | Stop | | (% | ID |
| Number | Site | Site | Site | Site | Sequence (5′ to 3′) | control) | NO |
| 1186532 | 8733 | 8750 | 14 | 31 | TAATAAAATCCCCAGGTA | 72 | 88 |
| 1186536 | 8737 | 8754 | 18 | 35 | AGAGTAATAAAATCCCCA | 103 | 89 |
| 1186540 | 8741 | 8758 | 22 | 39 | TCCCAGAGTAATAAAATC | 92 | 90 |
| 1186544 | 8745 | 8762 | 26 | 43 | TAATTCCCAGAGTAATAA | 119 | 91 |
| 1186548 | 8749 | 8766 | 30 | 47 | CACATAATTCCCAGAGTA | 100 | 92 |
| 1186552 | 8753 | 8770 | 34 | 51 | AGAACACATAATTCCCAG | 54 | 93 |
| 1186556 | 8757 | 8774 | 38 | 55 | GGGCAGAACACATAATTC | 80 | 94 |
| 1186560 | 8761 | 8778 | 42 | 59 | GATGGGGCAGAACACATA | 94 | 95 |
| 1186564 | 8765 | 8782 | 46 | 63 | GAGTGATGGGGCAGAACA | 98 | 96 |
| 1186568 | 8769 | 8786 | 50 | 67 | GAGAGAGTGATGGGGCAG | 89 | 97 |
| 1186572 | 8773 | 8790 | 54 | 71 | TTAAGAGAGAGTGATGGG | 134 | 98 |
| 1186576 | 8777 | 8794 | 58 | 75 | CCAATTAAGAGAGAGTGA | 109 | 99 |
| 1186580 | 8781 | 8798 | 62 | 79 | AAATCCAATTAAGAGAGA | 108 | 100 |
| 1186584 | 8785 | 8802 | 66 | 83 | TTAAAAATCCAATTAAGA | 118 | 101 |
| 1186588 | 8789 | 8806 | 70 | 87 | AATTTTAAAAATCCAATT | 124 | 102 |
| 1186592 | 8793 | 8810 | 74 | 91 | ATATAATTTTAAAAATCC | 118 | 103 |
| 1186596 | 8797 | 8814 | 78 | 95 | ATGAATATAATTTTAAAA | 92 | 104 |
| 1186600 | 8801 | 8818 | 82 | 99 | CAATATGAATATAATTTT | 101 | 105 |
| 1186604 | 8805 | 8822 | 86 | 103 | CCTGCAATATGAATATAA | 177 | 106 |
| 1186608 | 8809 | 8826 | 90 | 107 | GAGTCCTGCAATATGAAT | 132 | 107 |
| 1186612 | 8813 | 8830 | 94 | 111 | TGCCGAGTCCTGCAATAT | 95 | 108 |
| 1186616 | 8817 | 8834 | 98 | 115 | CTTCTGCCGAGTCCTGCA | 127 | 109 |
| 1186620 | 8821 | 8838 | 102 | 119 | AGGTCTTCTGCCGAGTCC | 181 | 110 |
| 1186624 | 8825 | 8842 | 106 | 123 | TCGAAGGTCTTCTGCCGA | 139 | 111 |
| 1186628 | 8829 | 8846 | 110 | 127 | TCTCTCGAAGGTCTTCTG | 147 | 112 |
| 1186632 | 8833 | 8850 | 114 | 131 | CCTTTCTCTCGAAGGTCT | 162 | 113 |
| 1186636 | 8837 | 8854 | 118 | 135 | TCTACCTTTCTCTCGAAG | 170 | 114 |
| 1186640 | 8841 | 8858 | 122 | 139 | ATTTTCTACCTTTCTCTC | 151 | 115 |
| 1186644 | 8845 | 8862 | 126 | 143 | TCTTATTTTCTACCTTTC | 198 | 116 |
| 1186648 | 8849 | 8866 | 130 | 147 | AAATTCTTATTTTCTACC | 160 | 117 |
| 1186652 | 8853 | 8870 | 134 | 151 | AGCCAAATTCTTATTTTC | 186 | 118 |
| 1186656 | 8857 | 8874 | 138 | 155 | AGAGAGCCAAATTCTTAT | 182 | 119 |
| 1186660 | 8861 | 8878 | 142 | 159 | ACACAGAGAGCCAAATTC | 171 | 120 |
| 1186664 | 8865 | 8882 | 146 | 163 | GCTCACACAGAGAGCCAA | 120 | 121 |
| 1186668 | 8869 | 8886 | 150 | 167 | ACATGCTCACACAGAGAG | 133 | 122 |
| 1186672 | 8873 | 8890 | 154 | 171 | GCACACATGCTCACACAG | 166 | 123 |
| 1186676 | 8877 | 8894 | 158 | 175 | ACACGCACACATGCTCAC | 161 | 124 |
| 1186680 | 8881 | 8898 | 162 | 179 | GCACACACGCACACATGC | 142 | 125 |
| 1186684 | 8885 | 8902 | 166 | 183 | TCTCGCACACACGCACAC | 125 | 126 |
| 1186688 | 8889 | 8906 | 170 | 187 | TCTCTCTCGCACACACGC | 136 | 127 |
| 1186692 | 8893 | 8910 | 174 | 191 | TCTCTCTCTCTCGCACAC | 145 | 128 |
| 1186696 | 8897 | 8914 | 178 | 195 | TCTGTCTCTCTCTCTCGC | 129 | 129 |
| 1186700 | 8901 | 8918 | 182 | 199 | GCTGTCTGTCTCTCTCTC | 155 | 130 |
| 1186704 | 8905 | 8922 | 186 | 203 | GCAGGCTGTCTGTCTCTC | 138 | 131 |
| 1186708 | 8909 | 8926 | 190 | 207 | TTAGGCAGGCTGTCTGTC | 118 | 132 |
| 1186712 | 8913 | 8930 | 194 | 211 | CTTCTTAGGCAGGCTGTC | 170 | 133 |
| 1186716 | 8917 | 8934 | 198 | 215 | ATTTCTTCTTAGGCAGGC | 188 | 134 |
| 1186720 | 8921 | 8938 | 202 | 219 | ATTCATTTCTTCTTAGGC | 193 | 135 |
| 1186724 | 8925 | 8942 | 206 | 223 | TCACATTCATTTCTTCTT | 196 | 136 |
| 1186728 | 8929 | 8946 | 210 | 227 | GCATTCACATTCATTTCT | 205 | 137 |
| 1186732 | 8933 | 8950 | 214 | 231 | AGCCGCATTCACATTCAT | 120 | 138 |
| 1186736 | 8937 | 8954 | 218 | 235 | CACAAGCCGCATTCACAT | 140 | 139 |
| 1186740 | 8941 | 8958 | 222 | 239 | GTGCCACAAGCCGCATTC | 120 | 140 |
| 1186744 | 8945 | 8962 | 226 | 243 | AACTGTGCCACAAGCCGC | 112 | 141 |
| 1186748 | 8949 | 8966 | 230 | 247 | TGTCAACTGTGCCACAAG | 149 | 142 |
| 1186752 | 8953 | 8970 | 234 | 251 | TCCTTGTCAACTGTGCCA | 151 | 143 |
| 1186756 | 8957 | 8974 | 238 | 255 | ATCATCCTTGTCAACTGT | 164 | 144 |
| 1186760 | 8961 | 8978 | 242 | 259 | ATTTATCATCCTTGTCAA | 166 | 145 |
| 1186764 | 8965 | 8982 | 246 | 263 | ATTGATTTATCATCCTTG | 123 | 146 |
| 1186768 | 8969 | 8986 | 250 | 267 | CATTATTGATTTATCATC | 97 | 147 |
| 1186772 | 8973 | 8990 | 254 | 271 | CTTGCATTATTGATTTAT | 60 | 148 |
| 1186776 | 8977 | 8994 | 258 | 275 | TAAGCTTGCATTATTGAT | 58 | 149 |
| 1186780 | 8981 | 8998 | 262 | 279 | ATAGTAAGCTTGCATTAT | 86 | 150 |
| 1186784 | 8985 | 9002 | 266 | 283 | AATGATAGTAAGCTTGCA | 60 | 151 |
| 1186788 | 8989 | 9006 | 270 | 287 | CATAAATGATAGTAAGCT | 75 | 152 |
| 1186792 | 8993 | 9010 | 274 | 291 | TATTCATAAATGATAGTA | 4 | 153 |
| 1186796 | 8997 | 9014 | 278 | 295 | TTGCTATTCATAAATGAT | 46 | 154 |
| 1186800 | 9001 | 9018 | 282 | 299 | AGTATTGCTATTCATAAA | 42 | 155 |
| 1186804 | 9005 | 9022 | 286 | 303 | CTTCAGTATTGCTATTCA | 50 | 156 |
| 1186808 | 9009 | 9026 | 290 | 307 | ATTTCTTCAGTATTGCTA | 97 | 157 |
| 1186812 | 9013 | 9030 | 294 | 311 | TTTAATTTCTTCAGTATT | 133 | 158 |
| 1186816 | 9017 | 9034 | 298 | 315 | TIGTTTTAATTICTTCAG | 141 | 159 |
| 1186820 | 9021 | 9038 | 302 | 319 | TCTTTTGTTTTAATTTCT | 90 | 160 |
| 1186824 | 9025 | 9042 | 306 | 323 | GCAATCTTTTGTTTTAAT | 88 | 161 |
| 1186828 | 9029 | 9046 | 310 | 327 | GACAGCAATCTTTTGTTT | 122 | 162 |
| 1186832 | 9033 | 9050 | 314 | 331 | TTGAGACAGCAATCTTTT | 128 | 163 |
| 1186836 | 9037 | 9054 | 318 | 335 | TATATTGAGACAGCAATC | 105 | 164 |
| 1186840 | 9041 | 9058 | 322 | 339 | AAGATATATTGAGACAGC | 124 | 165 |

TABLE 3
Percent control of human STMN2 RNA with uniformly MOE modified
oligonucleotides with phosphorothioate internucleoside linkages

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | STMN2 | SEQ |
| Compound | Start | Stop | Start | Stop | | (% | ID |
| Number | Site | Site | Site | Site | Sequence (5′ to 3′) | control) | NO |
| 1186533 | 8734 | 8751 | 15 | 32 | GTAATAAAATCCCCAGGT | 84 | 166 |
| 1186537 | 8738 | 8755 | 19 | 36 | CAGAGTAATAAAATCCCC | 117 | 167 |
| 1186541 | 8742 | 8759 | 23 | 40 | TTCCCAGAGTAATAAAAT | 73 | 168 |
| 1186545 | 8746 | 8763 | 27 | 44 | ATAATTCCCAGAGTAATA | 72 | 169 |
| 1186549 | 8750 | 8767 | 31 | 48 | ACACATAATTCCCAGAGT | 76 | 170 |
| 1186553 | 8754 | 8771 | 35 | 52 | CAGAACACATAATTCCCA | 78 | 171 |
| 1186557 | 8758 | 8775 | 39 | 56 | GGGGCAGAACACATAATT | 66 | 172 |
| 1186561 | 8762 | 8779 | 43 | 60 | TGATGGGGCAGAACACAT | 87 | 173 |
| 1186565 | 8766 | 8783 | 47 | 64 | AGAGTGATGGGGCAGAAC | 97 | 174 |
| 1186569 | 8770 | 8787 | 51 | 68 | AGAGAGAGTGATGGGGCA | 94 | 175 |
| 1186573 | 8774 | 8791 | 55 | 72 | ATTAAGAGAGAGTGATGG | 128 | 176 |
| 1186577 | 8778 | 8795 | 59 | 76 | TCCAATTAAGAGAGAGTG | 164 | 177 |
| 1186581 | 8782 | 8799 | 63 | 80 | AAAATCCAATTAAGAGAG | 145 | 178 |
| 1186585 | 8786 | 8803 | 67 | 84 | TTTAAAAATCCAATTAAG | 128 | 179 |
| 1186589 | 8790 | 8807 | 71 | 88 | TAATTTTAAAAATCCAAT | 117 | 180 |
| 1186593 | 8794 | 8811 | 75 | 92 | AATATAATTTTAAAAATC | 79 | 181 |
| 1186597 | 8798 | 8815 | 79 | 96 | TATGAATATAATTTTAAA | 83 | 182 |
| 1186601 | 8802 | 8819 | 83 | 100 | GCAATATGAATATAATTT | 143 | 183 |
| 1186605 | 8806 | 8823 | 87 | 104 | TCCTGCAATATGAATATA | 190 | 184 |
| 1186609 | 8810 | 8827 | 91 | 108 | CGAGTCCTGCAATATGAA | 149 | 185 |
| 1186613 | 8814 | 8831 | 95 | 112 | CTGCCGAGTCCTGCAATA | 189 | 186 |
| 1186617 | 8818 | 8835 | 99 | 116 | TCTTCTGCCGAGTCCTGC | 99 | 187 |
| 1186621 | 8822 | 8839 | 103 | 120 | AAGGTCTTCTGCCGAGTC | 168 | 188 |
| 1186625 | 8826 | 8843 | 107 | 124 | CTCGAAGGTCTTCTGCCG | 127 | 189 |
| 1186629 | 8830 | 8847 | 111 | 128 | TTCTCTCGAAGGTCTTCT | 232 | 190 |
| 1186633 | 8834 | 8851 | 115 | 132 | ACCTTTCTCTCGAAGGTC | 146 | 191 |
| 1186637 | 8838 | 8855 | 119 | 136 | TTCTACCTTTCTCTCGAA | 230 | 192 |
| 1186641 | 8842 | 8859 | 123 | 140 | TATTTTCTACCTTTCTCT | 163 | 193 |
| 1186645 | 8846 | 8863 | 127 | 144 | TTCTTATTTTCTACCTTT | 176 | 194 |
| 1186649 | 8850 | 8867 | 131 | 148 | CAAATTCTTATTTTCTAC | 133 | 195 |
| 1186653 | 8854 | 8871 | 135 | 152 | GAGCCAAATTCTTATTTT | 172 | 196 |
| 1186657 | 8858 | 8875 | 139 | 156 | CAGAGAGCCAAATTCTTA | 174 | 197 |
| 1186661 | 8862 | 8879 | 143 | 160 | CACACAGAGAGCCAAATT | 204 | 198 |
| 1186665 | 8866 | 8883 | 147 | 164 | TGCTCACACAGAGAGCCA | 134 | 199 |
| 1186669 | 8870 | 8887 | 151 | 168 | CACATGCTCACACAGAGA | 150 | 200 |
| 1186673 | 8874 | 8891 | 155 | 172 | CGCACACATGCTCACACA | 154 | 201 |
| 1186677 | 8878 | 8895 | 159 | 176 | CACACGCACACATGCTCA | 150 | 202 |
| 1186681 | 8882 | 8899 | 163 | 180 | CGCACACACGCACACATG | 148 | 203 |
| 1186685 | 8886 | 8903 | 167 | 184 | CTCTCGCACACACGCACA | 142 | 204 |
| 1186689 | 8890 | 8907 | 171 | 188 | CTCTCTCTCGCACACACG | 211 | 205 |
| 1186693 | 8894 | 8911 | 175 | 192 | GTCTCTCTCTCTCGCACA | 156 | 206 |
| 1186697 | 8898 | 8915 | 179 | 196 | GTCTGTCTCTCTCTCTCG | 119 | 207 |
| 1186701 | 8902 | 8919 | 183 | 200 | GGCTGTCTGTCTCTCTCT | 155 | 208 |
| 1186705 | 8906 | 8923 | 187 | 204 | GGCAGGCTGTCTGTCTCT | 112 | 209 |
| 1186709 | 8910 | 8927 | 191 | 208 | CTTAGGCAGGCTGTCTGT | 112 | 210 |
| 1186713 | 8914 | 8931 | 195 | 212 | TCTTCTTAGGCAGGCTGT | 170 | 211 |
| 1186717 | 8918 | 8935 | 199 | 216 | CATTTCTTCTTAGGCAGG | 193 | 212 |
| 1186721 | 8922 | 8939 | 203 | 220 | CATTCATTTCTTCTTAGG | 157 | 213 |
| 1186725 | 8926 | 8943 | 207 | 224 | TTCACATTCATTTCTTCT | 161 | 214 |
| 1186729 | 8930 | 8947 | 211 | 228 | CGCATTCACATTCATTTC | 228 | 215 |
| 1186733 | 8934 | 8951 | 215 | 232 | AAGCCGCATTCACATTCA | 164 | 216 |
| 1186737 | 8938 | 8955 | 219 | 236 | CCACAAGCCGCATTCACA | 171 | 217 |
| 1186741 | 8942 | 8959 | 223 | 240 | TGTGCCACAAGCCGCATT | 141 | 218 |
| 1186745 | 8946 | 8963 | 227 | 244 | CAACTGTGCCACAAGCCG | 135 | 219 |
| 1186749 | 8950 | 8967 | 231 | 248 | TTGTCAACTGTGCCACAA | 144 | 220 |
| 1186753 | 8954 | 8971 | 235 | 252 | ATCCTTGTCAACTGTGCC | 127 | 221 |
| 1186757 | 8958 | 8975 | 239 | 256 | TATCATCCTTGTCAACTG | 169 | 222 |
| 1186761 | 8962 | 8979 | 243 | 260 | GATTTATCATCCTTGTCA | 157 | 223 |
| 1186765 | 8966 | 8983 | 247 | 264 | TATTGATTTATCATCCTT | 113 | 224 |
| 1186769 | 8970 | 8987 | 251 | 268 | GCATTATTGATTTATCAT | 37 | 225 |
| 1186773 | 8974 | 8991 | 255 | 272 | GCTTGCATTATTGATTTA | 68 | 226 |
| 1186777 | 8978 | 8995 | 259 | 276 | GTAAGCTTGCATTATTGA | 63 | 227 |
| 1186781 | 8982 | 8999 | 263 | 280 | GATAGTAAGCTTGCATTA | 70 | 228 |
| 1186785 | 8986 | 9003 | 267 | 284 | AAATGATAGTAAGCTTGC | 63 | 229 |
| 1186789 | 8990 | 9007 | 271 | 288 | TCATAAATGATAGTAAGC | 7 | 230 |
| 1186793 | 8994 | 9011 | 275 | 292 | CTATTCATAAATGATAGT | 65 | 231 |
| 1186797 | 8998 | 9015 | 279 | 296 | ATTGCTATTCATAAATGA | 37 | 232 |
| 1186801 | 9002 | 9019 | 283 | 300 | CAGTATTGCTATTCATAA | 71 | 233 |
| 1186805 | 9006 | 9023 | 287 | 304 | TCTTCAGTATTGCTATTC | 94 | 234 |
| 1186809 | 9010 | 9027 | 291 | 308 | AATTTCTTCAGTATTGCT | 96 | 235 |
| 1186813 | 9014 | 9031 | 295 | 312 | TTTTAATTTCTTCAGTAT | 98 | 236 |
| 1186817 | 9018 | 9035 | 299 | 316 | TTTGTTTTAATTTCTTCA | 99 | 237 |
| 1186821 | 9022 | 9039 | 303 | 320 | ATCTTTTGTTTTAATTTC | 114 | 238 |
| 1186825 | 9026 | 9043 | 307 | 324 | AGCAATCTTTTGTTTTAA | 116 | 239 |
| 1186829 | 9030 | 9047 | 311 | 328 | AGACAGCAATCTTTTGTT | 130 | 240 |
| 1186833 | 9034 | 9051 | 315 | 332 | ATTGAGACAGCAATCTTT | 101 | 241 |
| 1186837 | 9038 | 9055 | 319 | 336 | ATATATTGAGACAGCAAT | 98 | 242 |
| 1186841 | 9042 | 9059 | 323 | 340 | TAAGATATATTGAGACAG | 95 | 243 |

TABLE 4
Percent control of human STMN2 RNA with uniformly MOE modified
oligonucleotides with phosphorothioate internucleoside linkages

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | STMN2 | SEQ |
| Compound | Start | Stop | Start | Stop | | (% | ID |
| Number | Site | Site | Site | Site | Sequence (5′ to 3′) | control) | NO |
| 1186534 | 8735 | 8752 | 16 | 33 | AGTAATAAAATCCCCAGG | 71 | 244 |
| 1186538 | 8739 | 8756 | 20 | 37 | CCAGAGTAATAAAATCCC | 65 | 245 |
| 1186542 | 8743 | 8760 | 24 | 41 | ATTCCCAGAGTAATAAAA | 86 | 246 |
| 1186546 | 8747 | 8764 | 28 | 45 | CATAATTCCCAGAGTAAT | 69 | 247 |
| 1186550 | 8751 | 8768 | 32 | 49 | AACACATAATTCCCAGAG | 76 | 248 |
| 1186554 | 8755 | 8772 | 36 | 53 | GCAGAACACATAATTCCC | 95 | 249 |
| 1186558 | 8759 | 8776 | 40 | 57 | TGGGGCAGAACACATAAT | 88 | 250 |
| 1186562 | 8763 | 8780 | 44 | 61 | GTGATGGGGCAGAACACA | 89 | 251 |
| 1186566 | 8767 | 8784 | 48 | 65 | GAGAGTGATGGGGCAGAA | 95 | 252 |
| 1186570 | 8771 | 8788 | 52 | 69 | AAGAGAGAGTGATGGGGC | 98 | 253 |
| 1186574 | 8775 | 8792 | 56 | 73 | AATTAAGAGAGAGTGATG | 121 | 254 |
| 1186578 | 8779 | 8796 | 60 | 77 | ATCCAATTAAGAGAGAGT | 164 | 255 |
| 1186582 | 8783 | 8800 | 64 | 81 | AAAAATCCAATTAAGAGA | 137 | 256 |
| 1186586 | 8787 | 8804 | 68 | 85 | TTTTAAAAATCCAATTAA | 99 | 257 |
| 1186590 | 8791 | 8808 | 72 | 89 | ATAATTTTAAAAATCCAA | 106 | 258 |
| 1186594 | 8795 | 8812 | 76 | 93 | GAATATAATTTTAAAAAT | 95 | 259 |
| 1186598 | 8799 | 8816 | 80 | 97 | ATATGAATATAATTTTAA | 81 | 260 |
| 1186602 | 8803 | 8820 | 84 | 101 | TGCAATATGAATATAATT | 154 | 261 |
| 1186606 | 8807 | 8824 | 88 | 105 | GTCCTGCAATATGAATAT | 166 | 262 |
| 1186610 | 8811 | 8828 | 92 | 109 | CCGAGTCCTGCAATATGA | 159 | 263 |
| 1186614 | 8815 | 8832 | 96 | 113 | TCTGCCGAGTCCTGCAAT | 129 | 264 |
| 1186618 | 8819 | 8836 | 100 | 117 | GTCTTCTGCCGAGTCCTG | 151 | 265 |
| 1186622 | 8823 | 8840 | 104 | 121 | GAAGGTCTTCTGCCGAGT | 208 | 266 |
| 1186626 | 8827 | 8844 | 108 | 125 | TCTCGAAGGTCTTCTGCC | 165 | 267 |
| 1186630 | 8831 | 8848 | 112 | 129 | TTTCTCTCGAAGGTCTTC | 176 | 268 |
| 1186634 | 8835 | 8852 | 116 | 133 | TACCTTTCTCTCGAAGGT | 123 | 269 |
| 1186638 | 8839 | 8856 | 120 | 137 | TTTCTACCTTTCTCTCGA | 152 | 270 |
| 1186642 | 8843 | 8860 | 124 | 141 | TTATTTTCTACCTTTCTC | 137 | 271 |
| 1186646 | 8847 | 8864 | 128 | 145 | ATTCTTATTTTCTACCTT | 191 | 272 |
| 1186650 | 8851 | 8868 | 132 | 149 | CCAAATTCTTATTTTCTA | 167 | 273 |
| 1186654 | 8855 | 8872 | 136 | 153 | AGAGCCAAATTCTTATTT | 172 | 274 |
| 1186658 | 8859 | 8876 | 140 | 157 | ACAGAGAGCCAAATTCTT | 172 | 275 |
| 1186662 | 8863 | 8880 | 144 | 161 | TCACACAGAGAGCCAAAT | 159 | 276 |
| 1186666 | 8867 | 8884 | 148 | 165 | ATGCTCACACAGAGAGCC | 126 | 277 |
| 1186670 | 8871 | 8888 | 152 | 169 | ACACATGCTCACACAGAG | 142 | 278 |
| 1186674 | 8875 | 8892 | 156 | 173 | ACGCACACATGCTCACAC | 170 | 279 |
| 1186678 | 8879 | 8896 | 160 | 177 | ACACACGCACACATGCTC | 122 | 280 |
| 1186682 | 8883 | 8900 | 164 | 181 | TCGCACACACGCACACAT | 133 | 281 |
| 1186686 | 8887 | 8904 | 168 | 185 | TCTCTCGCACACACGCAC | 145 | 282 |
| 1186690 | 8891 | 8908 | 172 | 189 | TCTCTCTCTCGCACACAC | 134 | 283 |
| 1186694 | 8895 | 8912 | 176 | 193 | TGTCTCTCTCTCTCGCAC | 116 | 284 |
| 1186698 | 8899 | 8916 | 180 | 197 | TGTCTGTCTCTCTCTCTC | 171 | 285 |
| 1186702 | 8903 | 8920 | 184 | 201 | AGGCTGTCTGTCTCTCTC | 141 | 286 |
| 1186706 | 8907 | 8924 | 188 | 205 | AGGCAGGCTGTCTGTCTC | 139 | 287 |
| 1186710 | 8911 | 8928 | 192 | 209 | TCTTAGGCAGGCTGTCTG | 127 | 288 |
| 1186714 | 8915 | 8932 | 196 | 213 | TTCTTCTTAGGCAGGCTG | 150 | 289 |
| 1186718 | 8919 | 8936 | 200 | 217 | TCATTTCTTCTTAGGCAG | 215 | 290 |
| 1186722 | 8923 | 8940 | 204 | 221 | ACATTCATTTCTTCTTAG | 185 | 291 |
| 1186726 | 8927 | 8944 | 208 | 225 | ATTCACATTCATTTCTTC | 174 | 292 |
| 1186730 | 8931 | 8948 | 212 | 229 | CCGCATTCACATTCATTT | 161 | 293 |
| 1186734 | 8935 | 8952 | 216 | 233 | CAAGCCGCATTCACATTC | 150 | 294 |
| 1186738 | 8939 | 8956 | 220 | 237 | GCCACAAGCCGCATTCAC | 122 | 295 |
| 1186742 | 8943 | 8960 | 224 | 241 | CTGTGCCACAAGCCGCAT | 210 | 296 |
| 1186746 | 8947 | 8964 | 228 | 245 | TCAACTGTGCCACAAGCC | 181 | 297 |
| 1186750 | 8951 | 8968 | 232 | 249 | CTTGTCAACTGTGCCACA | 153 | 298 |
| 1186754 | 8955 | 8972 | 236 | 253 | CATCCTTGTCAACTGTGC | 107 | 299 |
| 1186758 | 8959 | 8976 | 240 | 257 | TTATCATCCTTGTCAACT | 164 | 300 |
| 1186762 | 8963 | 8980 | 244 | 261 | TGATTTATCATCCTTGTC | 138 | 301 |
| 1186766 | 8967 | 8984 | 248 | 265 | TTATTGATTTATCATCCT | 100 | 302 |
| 1186770 | 8971 | 8988 | 252 | 269 | TGCATTATTGATTTATCA | 53 | 303 |
| 1186774 | 8975 | 8992 | 256 | 273 | AGCTTGCATTATTGATTT | 57 | 304 |
| 1186778 | 8979 | 8996 | 260 | 277 | AGTAAGCTTGCATTATTG | 70 | 305 |
| 1186782 | 8983 | 9000 | 264 | 281 | TGATAGTAAGCTTGCATT | 72 | 306 |
| 1186786 | 8987 | 9004 | 268 | 285 | TAAATGATAGTAAGCTTG | 46 | 307 |
| 1186790 | 8991 | 9008 | 272 | 289 | TTCATAAATGATAGTAAG | 37 | 308 |
| 1186794 | 8995 | 9012 | 276 | 293 | GCTATTCATAAATGATAG | 37 | 309 |
| 1186798 | 8999 | 9016 | 280 | 297 | TATTGCTATTCATAAATG | 44 | 310 |
| 1186802 | 9003 | 9020 | 284 | 301 | TCAGTATTGCTATTCATA | 62 | 311 |
| 1186806 | 9007 | 9024 | 288 | 305 | TTCTTCAGTATTGCTATT | 55 | 312 |
| 1186810 | 9011 | 9028 | 292 | 309 | TAATTTCTTCAGTATTGC | 82 | 313 |
| 1186814 | 9015 | 9032 | 296 | 313 | GTTTTAATTTCTTCAGTA | 101 | 314 |
| 1186818 | 9019 | 9036 | 300 | 317 | TTTTGTTTTAATTTCTTC | 104 | 315 |
| 1186822 | 9023 | 9040 | 304 | 321 | AATCTTTTGTTTTAATTT | 97 | 316 |
| 1186826 | 9027 | 9044 | 308 | 325 | CAGCAATCTTTTGTTTTA | 157 | 317 |
| 1186830 | 9031 | 9048 | 312 | 329 | GAGACAGCAATCTTTTGT | 120 | 318 |
| 1186834 | 9035 | 9052 | 316 | 333 | TATTGAGACAGCAATCTT | 97 | 319 |
| 1186838 | 9039 | 9056 | 320 | 337 | GATATATTGAGACAGCAA | 110 | 320 |
| 1186842 | 9043 | 9060 | 324 | 341 | ATAAGATATATTGAGACA | 100 | 321 |

### Example 2: Effect of Uniformly MOE Modified Oligonucleotides With Phosphorothioate Internucleoside Linkages on Human STMN2 In Vitro, Multiple Doses

Modified oligonucleotides selected from the example above were tested at various doses in CRISPR-edited SH-SY5Y cells (described hereinabove in Example 1). Cells were plated at a density of 20,000 cells per well and transfected using electroporation with 555 nM, 1,666 nM, 5,000 nM, and 15,000 nM concentrations of modified oligonucleotide, as specified in the tables below. After a treatment period of approximately 24 hours, total RNA was isolated from the cells and STMN2 RNA levels were measured by quantitative real-time PCR. Human STMN2 primer probe set RTS40280 (described hereinabove in Example 1) was used to measure RNA levels. STMN2 RNA levels were adjusted according to total RNA content, as measured by RiboGreen®. Results are presented in the tables below as percent control of the amount of STMN2 RNA, relative to untreated control cells. As illustrated in the tables below, STMN2 RNA levels were increased in a dose-dependent manner in modified oligonucleotide-treated cells. IC₅₀ was calculated using the “log(agonist) vs. response—variable slope (4 parameters)” formula using Prism6 software (Min=100, Max=249).

TABLE 5
Dose-dependent percent increase of human STMN2 RNA
by uniformly modified oligonucleotides

| | STMN2 (% control) | | | | |
|---|---|---|---|---|---|
| Compound | 555 | 1,666 | 5,000 | 15,000 | IC₅₀ |
| Number | nM | nM | nM | nM | (nM) |
| 1186604 | 127 | 146 | 187 | 206 | 4009 |
| 1186607 | 126 | 126 | 174 | 201 | 6053 |
| 1186627 | 103 | 130 | 176 | 202 | 5912 |
| 1186629 | 130 | 149 | 181 | 213 | 3814 |
| 1186644 | 97 | 154 | 166 | 194 | 6577 |
| 1186651 | 129 | 151 | 184 | 217 | 3479 |
| 1186652 | 139 | 186 | 198 | 223 | 1654 |
| 1186655 | 121 | 162 | 183 | 201 | 3956 |
| 1186656 | 135 | 188 | 225 | 239 | 1322 |
| 1186671 | 106 | 132 | 160 | 196 | 7706 |
| 1186712 | 136 | 145 | 171 | 209 | 4601 |
| 1186715 | 112 | 134 | 165 | 233 | 4889 |
| 1186716 | 141 | 174 | 198 | 212 | 2033 |
| 1186720 | 118 | 176 | 212 | 208 | 2191 |
| 1186723 | 135 | 165 | 203 | 213 | 2311 |
| 1186724 | 132 | 167 | 193 | 204 | 2858 |
| 1186727 | 76 | 171 | 195 | 230 | 2838 |
| 1186728 | 135 | 128 | 179 | 186 | 7285 |
| 1186751 | 113 | 122 | 140 | 150 | n.d.* |

*IC₅₀ above maximum dose tested

TABLE 6
Dose-dependent percent increase of human STMN2 RNA
by uniformly modified oligonucleotides

| | STMN2 expression (% control) | | | | |
|---|---|---|---|---|---|
| Compound | 555 | 1,666 | 5,000 | 15,000 | IC₅₀ |
| Number | nM | nM | nM | nM | (nM) |
| 1186605 | 126 | 148 | 183 | 229 | 3377 |
| 1186613 | 111 | 127 | 174 | 225 | 4889 |
| 1186622 | 113 | 140 | 170 | 205 | 5637 |
| 1186637 | 111 | 134 | 172 | 221 | 4899 |
| 1186645 | 150 | 172 | 197 | 235 | 1646 |
| 1186646 | 131 | 163 | 202 | 224 | 2314 |
| 1186655 | 176 | 186 | 197 | 217 | 596 |
| 1186657 | 124 | 157 | 175 | 142 | n/a* |
| 1186661 | 133 | 164 | 187 | 247 | 2330 |
| 1186670 | 146 | 152 | 166 | 193 | 6059 |
| 1186689 | 103 | 95 | 166 | 202 | 7529 |
| 1186698 | 122 | 116 | 154 | 182 | 11593 |
| 1186717 | 160 | 192 | 217 | 213 | 873 |
| 1186718 | 145 | 163 | 210 | 195 | 2255 |
| 1186722 | 123 | 180 | 193 | 220 | 2286 |
| 1186726 | 155 | 187 | 228 | 249 | 1024 |
| 1186729 | 148 | 171 | 209 | 231 | 1575 |
| 1186742 | 82 | 122 | 135 | 181 | 12831 |
| 1186746 | 132 | 156 | 166 | 209 | 4499 |

*IC₅₀ above maximum dose tested

### Example 3: Effect of Uniformly MOE Modified Oligonucleotides With Phosphorothioate Internucleoside Linkages on Human STMN2 In Vitro, Multiple Doses

Modified oligonucleotides selected from the examples above were tested at various doses in CRISPR-edited SH-SY5Y cells (described hereinabove in Example 1). Cells were plated at a density of 20,000 cells per well and transfected using electroporation with 555 nM, 1,666 nM, 5,000 nM, and 15,000 nM concentrations of modified oligonucleotide, as specified in the tables below. After a treatment period of approximately 24 hours, total RNA was isolated from the cells and STMN2 RNA levels were measured by quantitative real-time PCR. Human STMN2 primer probe set RTS41911 (forward sequence AAGGCAATCCTGCCTACTAAC, designated herein as SEQ ID NO: 6; reverse sequence GGTGGGTATCTGGTGATTCTTAG, designated herein as SEQ ID NO: 7; probe sequence TCCATCTGTGAAGCTGACGCAGTT, designated herein as SEQ ID NO: 8) was used to measure RNA levels. STMN2 RNA levels were adjusted according to total RNA content, as measured by RiboGreen®. Results are presented in the tables below as percent control of the amount of STMN2 RNA, relative to untreated control cells. As illustrated in the tables below, STMN2 RNA levels were increased in a dose-dependent manner in modified oligonucleotide-treated cells. IC₅₀ was calculated using the “log(agonist) vs. response—variable slope (4 parameters)” formula using Prism6 software (Min=100, Max=249).

TABLE 7
Dose-dependent percent increase of human STMN2 RNA
by uniformly modified oligonucleotides

| | STMN2 (% control) | | | | |
|---|---|---|---|---|---|
| Compound | 555 | 1,666 | 5,000 | 15,000 | IC₅₀ |
| Number | nM | nM | nM | nM | (nM) |
| 1186604 | 114 | 144 | 165 | 202 | 6077 |
| 1186607 | 107 | 109 | 137 | 187 | 11626 |
| 1186627 | 96 | 120 | 154 | 188 | 9775 |
| 1186629 | 125 | 150 | 177 | 202 | 4710 |
| 1186644 | 130 | 160 | 159 | 198 | 5939 |
| 1186651 | 125 | 139 | 171 | 205 | 5411 |
| 1186652 | 148 | 175 | 191 | 196 | 2296 |
| 1186655 | 128 | 156 | 220 | 195 | 2610 |
| 1186656 | 129 | 173 | 200 | 200 | 2587 |
| 1186671 | 100 | 116 | 129 | 166 | n/a* |
| 1186712 | 121 | 116 | 159 | 193 | 8686 |
| 1186715 | 100 | 124 | 156 | 211 | 6931 |
| 1186716 | 136 | 148 | 174 | 189 | 6209 |
| 1186720 | 111 | 170 | 194 | 205 | 3150 |
| 1186723 | 118 | 145 | 161 | 203 | 6204 |
| 1186724 | 145 | 161 | 188 | 203 | 2924 |
| 1186727 | 128 | 162 | 190 | 212 | 3011 |
| 1186728 | 124 | 120 | 168 | 171 | 13516 |
| 1186751 | 112 | 121 | 146 | 130 | n.d.** |

*IC₅₀ above maximum dose tested
**IC₅₀ cannot be calculated

TABLE 8
Dose-dependent percent increase of human STMN2 RNA
by uniformly modified oligonucleotides

| | STMN2 (% control) | | | | |
|---|---|---|---|---|---|
| Compound | 555 | 1,666 | 5,000 | 15,000 | IC₅₀ |
| Number | nM | nM | nM | nM | (nM) |
| 1186605 | 123 | 134 | 199 | 231 | 3304 |
| 1186613 | 113 | 124 | 181 | 228 | 4516 |
| 1186622 | 137 | 157 | 207 | 223 | 2254 |
| 1186637 | 111 | 139 | 192 | 238 | 3430 |
| 1186645 | 118 | 149 | 179 | 210 | 4313 |
| 1186646 | 140 | 177 | 208 | 238 | 1594 |
| 1186655 | 158 | 191 | 215 | 227 | 970 |
| 1186657 | 130 | 159 | 185 | 196 | 4042 |
| 1186661 | 141 | 182 | 208 | 238 | 1481 |
| 1186670 | 129 | 157 | 175 | 200 | 4558 |
| 1186689 | 108 | 135 | 180 | 212 | 4833 |
| 1186698 | 113 | 122 | 172 | 181 | 9174 |
| 1186717 | 143 | 190 | 205 | 214 | 1433 |
| 1186718 | 150 | 170 | 230 | 208 | 1402 |
| 1186722 | 119 | 159 | 192 | 203 | 3553 |
| 1186726 | 137 | 169 | 205 | 214 | 2087 |
| 1186729 | 147 | 180 | 208 | 215 | 1503 |
| 1186742 | 113 | 124 | 145 | 184 | 11785 |
| 1186746 | 135 | 149 | 179 | 206 | 4126 |

1. (canceled)

2. An oligomeric compound, comprising a modified oligonucleotide consisting of 12 to 50 linked nucleosides and having a nucleobase sequence comprising at least 8, at least 9, at least 10, at least 11, at least 12, at least 13, at least 14, at least 15, at least 16, at least 17, or at least 18 consecutive nucleobases of any of the nucleobase sequences of SEQ ID NOs: 34, 35, 112, 113, 190, 191, 267, and 268, wherein the modified oligonucleotide comprises at least one modified internucleoside linkage and/or at least one modified nucleoside comprising a modified sugar moiety.

3. An oligomeric compound, comprising a modified oligonucleotide consisting of 12 to 50 linked nucleosides and having a nucleobase sequence comprising at least 8, at least 9, at least 10, at least 11, at least 12, at least 13, at least 14, at least 15, at least 16, at least 17, or at least 18 consecutive nucleobases complementary to:
8827-8851 of SEQ ID NO: 1 or 108-132 of SEQ ID NO: 2.

4. (canceled)

5. The oligomeric compound of claim 3, wherein the modified oligonucleotide is a single-stranded modified oligonucleotide.

6. The oligomeric compound of claim 3, wherein at least one internucleoside linkage of the modified oligonucleotide is a modified internucleoside linkage.

7. The oligomeric compound of claim 6, wherein the modified internucleoside linkage is a phosphorothioate internucleoside linkage.

8. The oligomeric compound of claim 3, wherein each internucleoside linkage of the modified oligonucleotide is a modified internucleoside linkage.

9. The oligomeric compound of claim 8, wherein each modified internucleoside linkage of the modified oligonucleotide is a phosphorothioate internucleoside linkage.

10. The oligomeric compound of claim 3, wherein at least one internucleoside linkage of the modified oligonucleotide is a phosphodiester internucleoside linkage.

11. The oligomeric compound of claim 3, wherein each internucleoside linkage of the modified oligonucleotide is either a phosphodiester internucleoside linkage or a phosphorothioate internucleoside linkage.

12. The oligomeric compound of claim 3, wherein at least one nucleobase of the modified oligonucleotide comprises a modified nucleobase.

13. The oligomeric compound of claim 12, wherein the modified nucleobase is a 5-methylcytosine.

14. The oligomeric compound of claim 3, wherein the modified oligonucleotide comprises at least 1 modified nucleoside comprising a modified sugar moiety.

15. The oligomeric compound of claim 14, wherein the modified sugar moiety is bicyclic sugar moiety.

16. The oligomeric compound of claim 15, wherein the bicyclic sugar moiety comprises a 2′-4′ bridge selected from —O—CH₂—; and —O—CH(CH₃)—.

17. The oligomeric compound of claim 3, wherein the modified oligonucleotide comprises at least 1 modified nucleoside comprising a non-bicyclic sugar moiety.

18. The oligomeric compound of claim 17, wherein the non-bicyclic sugar moiety is 2′-MOE sugar moiety or a 2′-OMe sugar moiety.

19. (canceled)

20. The oligomeric compound of claim 3, wherein the modified oligonucleotide comprises at least 1 modified nucleoside comprising a sugar surrogate.

21. The oligomeric compound of claim 20, wherein the sugar surrogate is morpholino or PNA.

22. (canceled)

23. The oligomeric compound of claim 3, wherein the modified oligonucleotide has a sugar motif comprising:
a 5′-region consisting of 1-6 linked 5′-nucleosides;
a central region consisting of 6-10 linked central region nucleosides; and
a 3′-region consisting of 1-6 linked 5′-nucleosides; wherein each of the 5′-region nucleosides and each of the 3′-region nucleosides comprises a modified sugar moiety and each of the central region nucleosides comprises a 2′-deoxynucleoside sugar moiety.

24. The oligomeric compound of claim 3, wherein the modified oligonucleotide consists of 20 linked nucleosides and has the following internucleoside motif: sooosssssssssssooss; wherein,
s=a phosphorothioate internucleoside linkage, and
o=a phosphodiester internucleoside linkage.

25. The oligomeric compound of claim 3, wherein the modified oligonucleotide consists of 12-18, 12-20, 14-20, 16-20, or 17-19 linked nucleosides.

26. The oligomeric compound of claim 3, wherein the modified oligonucleotide consists of 16, 17, or 18 linked nucleosides.

27. The oligomeric compound of claim 3 consisting of the modified oligonucleotide.

28. The oligomeric compound of claim 3, comprising a conjugate group comprising a conjugate moiety and a conjugate linker.

29.-37. (canceled)

38. An oligomeric duplex comprising the oligomeric compound of claim 3.

39. An antisense compound comprising or consisting of the oligomeric compound of claim 3.

40. A modified oligonucleotide consisting of 12 to 50 linked nucleosides and having a nucleobase sequence comprising at least 8, at least 9, at least 10, at least 11, at least 12, at least 13, at least 14, at least 15, at least 16, at least 17, or at least 18 consecutive nucleobases of any of the nucleobase sequences of SEQ ID NOs: 34, 35, 112, 113, 190, 191, 267, and 268, wherein the modified oligonucleotide comprises at least one modified internucleoside linkage and/or at least one modified nucleoside comprising a modified sugar moiety.

41. A pharmaceutical composition comprising the oligomeric compound of claim 3 and at least one pharmaceutically acceptable carrier or diluent.

42.-49. (canceled)

50. The pharmaceutical composition of claim 41, wherein the pharmaceutically acceptable diluent is artificial cerebrospinal fluid.

51. A pharmaceutical composition comprising the oligomeric compound of claim 2 and at least one pharmaceutically acceptable carrier or diluent.

52. The pharmaceutical composition of claim 51, wherein the pharmaceutically acceptable diluent is artificial cerebrospinal fluid.
